SEQUENCE LISTING (83 sequences)

SEQ ID NO: 1 - DNA, 558 bp, Artificial sequence
  synthetic sequence
atgatgacaccagaaaacgacgaagagcagacatctgtgttctccgctactgtttacggagacaaaattcaaggaaagaataaacgcaaacgcgtgattggtctatgtattagaatatctatggttatttcactactatctatgattaccatgtccgcgtttctcatagtgcgcctaaatcaatgcatgtctgctaacgaggctgctattactgacgccgctgttgccgttgctgctgcatcatctactcatagaaaggttgcgtctagcactacgcaatatgatcacaaagaaagctgtaatggtttatattaccagggttcttgttatatattacattcagactaccagttattctcggatgctaaagcaaattgcactgcggaatcatcaacactacccaataaatccgatgtcttgattacctggctcattgattatgttgaggatacatggggatctgatggtaatccaattacaaaaactacatccgattatcaagattctgatgtatcacaagaagttagaaagtatttttgtgttaaaacaatgaactaa

SEQ ID NO: 2 - DNA, 558 bp, Artificial sequence
  synthetic sequence
atgatgacaccagaaaacgacgaagagcagacatctgtgttctccgctactgtttacagagacaaaattcagggaaagaataaacgcaaacgcgtgattggtctatgtattagaatatctatggttatttcactactatctatgattaccatgtccgcgtttctcatagtgcgcctaaatcaatgcatgtctgctaacgaggctgctattactgacgccgctgttgccgttgctgctgcatcatctactcatagaaaggttgcgtctagcactacgcaatatgatcacaaagaaagctgtaatggtttatattaccagggttcttgttatatattacattcagactaccagttattctcggatgctaaagcaaattgcactgcggaatcatcaacactacccaataaatccgatgtcttgactacctggctcattgattatgttaaggatacatggggatctgatggtaatccaattacaaaaactacatccgattatcaagattctgatgtatcacaagaagttagaaagtatttttgtgttaaaacaatgaactaa

SEQ ID NO: 3 - DNA, 558 bp, Artificial sequence
  synthetic sequence
atgatgacaccagaaaacgacgaagagcagacatctgtgttctccgctactgtttacggagacaaaattcagggaaagaataaacgcaaacgcgtgattggtctatgtattagaatatctatggttatttcactactatctatgattaccatgtccgcgtttctcatagtgcgcctaaatcaatgcatgtctgctaacgaggctgctattactgacgccgctgttgccgttgctgctgcatcatctactcatagaaaggttgcgtctagcactacgcaatatgatcacaaagaaagctgtaatggtttatattaccagggttcttgttatatattacattcagactaccagttattctcggatgctaaagcaaattgcactgcggaatcatcaacactacccaataaatccgatgtcttgattacctggctcattgattatgttgaggatacatggggatctgatggtaatccaattacaaaaactacatccgattatcaagattctgatatatcacaagaagttagaaagtatttttgtgttaaaacaatgaactaa

SEQ ID NO: 4 - DNA, 558 bp, Artificial sequence
  synthetic sequence
atgatgacaccagaaaacgacgaagagcagacatctgtgttctccgctactgtttacggagacaaaattcagggaaagaataaacgcaaacgcgtgattggtctatgtattagaatatctatggttatttcactactatctatgattaccatgtccgcgtttctcatagtgcgcctaaatcaatgcatgtctgctaacgaggctgctattactgacgccgctgttgccgttgctgctgcatcatctactcatagaaaggttgcgtctagcactacacaatatgatcacaaagaaagctgtaatggtttatattaccagggttcttgttatatattacattcagactaccagttattctcggatgctaaagcaaattgcactgcggaatcatcaacactacccaataaatccgatgtcttgactacctggctcattgattatgttgaggatacatggggatctgatggtaatccaattacaaaaactacatccgattatcaagattctgatgtatcacaagaagttagaaagtatttttgtgttaaaacaatgaactaa

SEQ ID NO: 5 - DNA, 558 bp, Artificial sequence
  synthetic sequence
atgatgacaccagaaaacgacgaagagcagacatctgtgttctccgctactgtttacggagacaaaattcagggaaagaataaacgcaaacgcgtgattggtctatgtattagaatatctatggttatttcactactatctatgattaccatgtcagcgtttctcatagtgcgcctaaatcaatgcatgtctgctaacgaggctgctattactgacgccgctgttgccgttgctgctgcatcatctactcatagaaaggttgcgtctagcactacacaatatgatcacaaagaaagctgtaatggtttatattaccagggttcttgttatatattacattcagactaccagttattctcggatgctaaagcaaattgcactgcggaatcatcaacactacccaataaatccgatgtcttgattacctggctcattgattatgttgaggatacatggggatctgatggtaatccaattacaaaaactacatccgattatcaagattctgatgtatcacaagaagttagaaagtatttttgtgttaaaacaatgaactaa

SEQ ID NO: 6 - DNA, 558 bp, Artificial sequence
  synthetic sequence
atgatgacaccagaaaacgacgaagagcagacatctgtgttctccgctactgtttacggagacaaaattcaaggaaagaataaacgcaaacgcgtgattggtctatgtattagaatatctatggttatttcactactatctatgattaccatgtccgcgtttctcatagtgcgcctaaatcaatgcatgtctgctaacgaggctgctattactgacgccgctgttgccgttgctgctgcatcatctactcatagaaaggttgcgtctagcactacacaatatgatcacaaagaaagctgtaatggtttatattaccagggttcttgttatatattacattcagactaccagttattctcggatgctaaagcaaattgcactgcggaatcatcaacactacccaataaatccgatgtcttgattacctggctcattgattatgttgaggatacatggggatctgatggtaatccaattacaaaaactacatccgattatcaagattctgatgtatcacaagaagttagaaagtatttttgtgttaaaacaatgaactaa

SEQ ID NO: 7 - DNA, 558 bp, Artificial sequence
  synthetic sequence
atgatgacaccagaaaacgacgaagagcagacatctgtgttctccgctactgtttacggagacaaaattcagggaaagaataaacgcaaacgcgtgattggtctatgtattagaatatctatggttatttcactactatctatgattaccatgtccgcgtttctcatagtgcgcctaaatcaatgcatgtctgctaacgaggctgctattactgacgccgctgttgccgttgctgctgcatcatctactcatagaaaggttgcgtctagcactacacaatatgatcacaaagaaagctgtaatggtttatattaccagggttcttgttatatattacattcagactaccagttattctcggatgctaaagcaaattgcactgcggaatcatcaacactacccaataaatccgatgtcttgattacctggctcattgattatgttgaggatacatggggatctgatggtaatccaattacaaaaactacatccgattatcaagattctgatgtatcacaagaagttagaaagtatttttgtgttaaaacaatgaactaa

SEQ ID NO: 8 - protein, 185 aa, Artificial sequence
  synthetic sequence
MMTPENDEEQTSVFSATVYGDKIQGKNKRKRVIGLCIRISMVISLLSMITMSAFLIVRLNQCMSANEAAITDAAVAVAAASSTHRKVASSTTQYDHKESCNGLYYQGSCYILHSDYQLFSDAKANCTAESSTLPNKSDVLITWLIDYVEDTWGSDGNPITKTTSDYQDSDVSQEVRKYFCVKTMN

SEQ ID NO: 9 - protein, 185 aa, Artificial sequence
  synthetic sequence
MMTPENDEEQTSVFSATVYRDKIQGKNKRKRVIGLCIRISMVISLLSMITMSAFLIVRLNQCMSANEAAITDAAVAVAAASSTHRKVASSTTQYDHKESCNGLYYQGSCYILHSDYQLFSDAKANCTAESSTLPNKSDVLTTWLIDYVKDTWGSDGNPITKTTSDYQDSDVSQEVRKYFCVKTMN

SEQ ID NO: 10 - protein, 185 aa, Artificial sequence
  synthetic sequence
MMTPENDEEQTSVFSATVYGDKIQGKNKRKRVIGLCIRISMVISLLSMITMSAFLIVRLNQCMSANEAAITDAAVAVAAASSTHRKVASSTTQYDHKESCNGLYYQGSCYILHSDYQLFSDAKANCTAESSTLPNKSDVLITWLIDYVEDTWGSDGNPITKTTSDYQDSDISQEVRKYFCVKTMN

SEQ ID NO: 11 - protein, 185 aa, Artificial sequence
  synthetic sequence
MMTPENDEEQTSVFSATVYGDKIQGKNKRKRVIGLCIRISMVISLLSMITMSAFLIVRLNQCMSANEAAITDAAVAVAAASSTHRKVASSTTQYDHKESCNGLYYQGSCYILHSDYQLFSDAKANCTAESSTLPNKSDVLTTWLIDYVEDTWGSDGNPITKTTSDYQDSDVSQEVRKYFCVKTMN

SEQ ID NO: 12 - protein, 185 aa, Artificial sequence; identical to SEQ ID NO: 8
  synthetic sequence

SEQ ID NO: 13 - protein, 185 aa, Artificial sequence; identical to SEQ ID NO: 8
  synthetic sequence

SEQ ID NO: 14 - protein, 185 aa, Artificial sequence; identical to SEQ ID NO: 8
  synthetic sequence

SEQ ID NO: 15 - DNA, 507 bp, Artificial sequence
  synthetic sequence
atgaaatcgcttaatagacaaactgtaagtatgtttaagaagttgtcggtgccggccgctataatgatgatactctcaaccattattagtggcataggaacatttctgcattacaaagaagaactgatgcctagtgcttgcgccaatggatggatacaatacgataaacattgttatctagataccaacattaaaatgtccacagataatgcggtttatcagtgtcgtaaattacgagctagattgcctagacctgatactagacatctgagagtattgtttagtattttttataaagattattgggtaagtttaaaaaagaccaataataaatggttagatattaataatgataaagatatagatattagtaaattaacaaattttaaacaactaaacagtacgacggatgctgaagcgtgttatatatacaagtctggaaaactggttaaaacagtatgtaaaagtactcaatctgtactatgtgttaaaaaattctacaagtga

SEQ ID NO: 16 - DNA, 507 bp, Artificial sequence
  synthetic sequence
atgaaatcgcttaatagacaaactgtaagtaggtttaagaagttgtcggtgccggtcgctataatgatgatactctcaaccattattagtggcataggaacatttctgcattacaaagaagaactgatgcctagtgcttgcgccaatggatggatacaatacgataaacattgttatttagatactaacattaaaatgtctacagataatgcggtttatcagtgtcgtaaattacgagccagattgcctagaccggatactagacatctgagagtattgtttagtattttttataaagattattgggtaagtttaaaaaagaccaatgataaatggttagatattaataatgataaagatatagatattagtaaattaacaaattttaaacaactaaacagtacgacggatgctgaagcgtgttatatatacaagtctggaaaactggttaaaacagtatgtaaaagtactcaatctgtactatgtgttaaaaaattctacaagtga

SEQ ID NO: 17 - DNA, 507 bp, Artificial sequence
  synthetic sequence
atgaaatcgcttaatagacaaactgtaagtaggtttaagaagttgtcggtgccggccgctataatgatgatactctcaaccattattagtggcataggaacatttctgcattacaaagaagaactgatgcctagtgcttgcgccaatggatggatacaatacgataaacattgttatctagataccaacattaaaatgtctacagataatgcggtttatcagtgtcgtaaattacgagctagattgcctagacctgatactagacatctgagagtattgtttagtattttttataaagattattgggtaagtttaaaaaagaccaataataaatggttagatattaataatgataaagatatagatattagtaaattaacaaattttaaacaactaaacagtacgacggatgctgaagcgtgttatatatacaagtctggaaaactggttgaaacagtatgtaaaagtactcaatctgtactatgtgttaaaaaattctacaagtga

SEQ ID NO: 18 - DNA, 507 bp, Artificial sequence
  synthetic sequence
atgaaatcgcttaatagacaaactgtaagtaggtttaagaagttgtcggtgccggccgctataatgatgatactctcaaccattattagtggcataggaacatttctgcattacaaagaagaactgatgcctagtgcttgcgccaatggatggatacaatacgataaacattgttatttagatactaacattaaaatgtctacagataatgcggtttatcagtgtcgtaaattacgagccagattgcctagacctgatactagacatctgagagtattgtttagtattttttataaagattattgggtaagtttaaaaaagaccaataataaatggttagatattaataatgataaagatatagatattagtaaattaacaaattttaaacaactaaacagtacgacggatgctgaagcgtgttatatatacaagtctggaaaactggttaaaacagtatgtaaaagtactcaatctgtactatgtgttaaaaaattctacaagtga

SEQ ID NO: 19 - DNA, 507 bp, Artificial sequence
  synthetic sequence
atgaaatcgcttaatagacaaactgtaagtaggtttaagaagttgtcggtgccggccgctataatgatgatactctcaaccattattagcggcataggaacatttctgcattacaaagaagaactgatgcctagtgcttgcgccaatggatggatacaatacgataaacattgttatttagatactaacattaaaatgtctacagataatgcggtttatcagtgtcgtaaattacgagccagattgcctagaccggatactagacatctgagagtattgtttagtattttttataaagattattgggtaagtttaaaaaagaccaatgataaatggttagatattaataatgataaagatatagatattagtaaattaacaaattttaaacaactaaacagtacgacggatgctgaagcgtgttatatatacaagtctggaaaactggttaaaacagtatgtaaaagtactcaatctgtactatgtgttaaaaaattctacaagtga

SEQ ID NO: 20 - DNA, 507 bp, Artificial sequence
  synthetic sequence
atgaaatcgcttaatagacaaactgtaagtaggtttaagaagttgtcggtgccggccgctataatgatgatactctcaaccattattagtggcataggaacatttctgcattacaaagaagaactgatgcctagtgcttgcgccaatggatggatacaatacgataaacattgttatttagatactaacattaaaatgtctacagataatgcggtttatcagtgtcgtaaattacgagccagattgcctagaccggatactagacatctgagagtattgtttagtattttttataaagattattgggtaagtttaaaaaagaccaatgataaatggttagatattaataatgataaagatatagatattagtaaattaacaaattttaaacaactaaacagtacgacggatgctgaagcgtgttatatatacaagtctggaaaactggttaaaacagtatgtaaaagtactcaatctgtactatgtgttaaaaaattctacaagtga

SEQ ID NO: 21 - DNA, 507 bp, Artificial sequence
  synthetic sequence
atgaaatcgcttaatagacaaactgtaagtaggtttaagaagttgtcggtgccggccgctataatgatgatactctcaaccattattagtggcataggaacatttctgcattacaaagaagaactgatgcctagtgcttgcgccaatggatggatacaatacgataaacattgttatttagatactaacattaaaatgtctacagataatgcggtttatcagtgtcgtaaattacgagccagattgcctagaccggatactagacatctgagagtattgtttagtattttttataaagattattgggtaagtttaaaaaagaccaataataaatggttagatattaataatgataaagatatagatattagtaaattaacaaattttaaacaactaaacagtacgacggatgctgaagcgtgttatatatacaagtctggaaaactggttaaaacagtatgtaaaagtactcaatctgtactatgtgttaaaaaattctacaagtga

SEQ ID NO: 22 - protein, 168 aa, Artificial sequence
  synthetic sequence
MKSLNRQTVSMFKKLSVPAAIMMILSTIISGIGTFLHYKEELMPSACANGWIQYDKHCYLDTNIKMSTDNAVYQCRKLRARLPRPDTRHLRVLFSIFYKDYWVSLKKTNNKWLDINNDKDIDISKLTNFKQLNSTTDAEACYIYKSGKLVKTVCKSTQSVLCVKKFYK

SEQ ID NO: 23 - protein, 168 aa, Artificial sequence
  synthetic sequence
MKSLNRQTVSRFKKLSVPVAIMMILSTIISGIGTFLHYKEELMPSACANGWIQYDKHCYLDTNIKMSTDNAVYQCRKLRARLPRPDTRHLRVLFSIFYKDYWVSLKKTNDKWLDINNDKDIDISKLTNFKQLNSTTDAEACYIYKSGKLVKTVCKSTQSVLCVKKFYK

SEQ ID NO: 24 - protein, 168 aa, Artificial sequence
  synthetic sequence
MKSLNRQTVSRFKKLSVPAAIMMILSTIISGIGTFLHYKEELMPSACANGWIQYDKHCYLDTNIKMSTDNAVYQCRKLRARLPRPDTRHLRVLFSIFYKDYWVSLKKTNNKWLDINNDKDIDISKLTNFKQLNSTTDAEACYIYKSGKLVETVCKSTQSVLCVKKFYK

SEQ ID NO: 25 - protein, 168 aa, Artificial sequence
  synthetic sequence
MKSLNRQTVSRFKKLSVPAAIMMILSTIISGIGTFLHYKEELMPSACANGWIQYDKHCYLDTNIKMSTDNAVYQCRKLRARLPRPDTRHLRVLFSIFYKDYWVSLKKTNNKWLDINNDKDIDISKLTNFKQLNSTTDAEACYIYKSGKLVKTVCKSTQSVLCVKKFYK

SEQ ID NO: 26 - protein, 168 aa, Artificial sequence
  synthetic sequence
MKSLNRQTVSRFKKLSVPAAIMMILSTIISGIGTFLHYKEELMPSACANGWIQYDKHCYLDTNIKMSTDNAVYQCRKLRARLPRPDTRHLRVLFSIFYKDYWVSLKKTNDKWLDINNDKDIDISKLTNFKQLNSTTDAEACYIYKSGKLVKTVCKSTQSVLCVKKFYK

SEQ ID NO: 27 - protein, 168 aa, Artificial sequence; identical to SEQ ID NO: 26
  synthetic sequence

SEQ ID NO: 28 - protein, 168 aa, Artificial sequence; identical to SEQ ID NO: 25
  synthetic sequence

SEQ ID NO: 29 - DNA, 948 bp, Artificial sequence
  synthetic sequence
atgacacgattaccaatacttttgttactaatatcattagtatacgctacaccttttcctcagacatctaaaaaaataggtgatgatgcaactctatcatgtaatcgaaataatacaaatgactacgttgttatgagtgcttggtataaggagcccaattccattattcttttagctgctaaaagcgacgtcttgtattttgataattataccaaggataaaatatcttacgactctccatacgatgatctagttacaactatcacaattaaatcattgactgctagagatgccggtacttatgtatgtgcattctttatgacatcgcctacaaatgacactgataaagtagattatgaagaatactccacagagttgattgtaaatacagatagtgaatcgactatagacataatactatctggatctacacattcaccagaaactagttctgagaaacctgattatatagataattctaattgctcgtcggtattcgaaatcgcgactccggaaccaattactgataatgtagaagatcatacagacaccgtcacatacactagtgatagcattaatacagtaagtgcaacatctggagaatccacaacagacgagactccggaaccaattactgataaagaagaagatcatacagtcacagacactgtctcatacactacagtaagtacatcatctggaattgtcactactaaatcaaccaccgatgatgcggatctttatgatacgtacaatgataatgatacagtaccatcaactactgtaggtagtagtacaacctctattagtaattataaaaccaaggactttgtagaaatatttggtattaccgcattaattatattgtcggccgtggcaatattctgtattacgtattatatatgtaataaacgttcacgtaaatacaaaacagagaacaaagtctag

SEQ ID NO: 30 - DNA, 948 bp, Artificial sequence
  synthetic sequence
atgacacgattaccaatacttttgttactaatatcattagtatacgctacaccttttcctcagacatctaaaaaaataggtgatgatgcaactttatcatgtaatcgaaataatacaaatgactacgttgttatgagtgcttggtataaggagcccaattccattattcttttagctgctaaaagcgacgtcttgtattttgataattataccaaggataaaatatcttacgactctccatacgatgatctagttacaactatcacaattaaatcattgactgctagagatgccggtacttatgtatgtgcattctttatgacatcgcctacaaatgacactgataaagtagattatgaagaatactccacagagttgattgtaaatacagatagtgaatcgactatagacataatactatctggatctacacattcaccggaaactagttctgagaaacctgattatatagataattctaattgctcgtcggtattcgaaatcgcgactccggaaccaattactgataatgtagaagatcatacagacaccgtcacatacactagtgatagcattaatacagtaagtgcatcatctggagaatccacaacagacgagactccggaaccaattactgataaagaagaagatcatacagttacagacactgtctcatacactacagtaagtacatcatctggaattgtcactactaaatcaaccaccgatgatgcggatctttatgatacgtacaatgataatgatacagtaccatcaactactgtaggcggtagtacaacctctattagcaattataaaaccaaggactttgtagaaatatttggtattaccgcattaattatattgtcggccgtggcaatattctgtattacatattatatatataataaacgttcacgtaaatacaaaacagagaacaaagtctag

SEQ ID NO: 31 - DNA, 948 bp, Artificial sequence
  synthetic sequence
atgacacgattaccaatacttttgttactaatatcattagtatacgctacaccttttcctcagacatctaaaaaaataggtgatgatgcaactctatcatgtaatcgaaataatacaaatgactacgttgttatgagtgcttggtataaggagcccaattccattattcttttagctgctaaaagcgacgtcttgtattttgataattataccaaggataaaatatcttacgactctccatacgatgatctagttacaactatcacaattaaatcattgactgctagagatgccggtacttatgtatgtgcattctttatgacatcaactacaaatgacactgataaagtagattatgaagaatactccacagagttgattgtaaatacagatagtgaatcgactatagacataatactatctggatctacacattcaccagaaactagttctgagaaaccagaggatatagataattttaattgctcgtcggtattcgaaatcgcgactccggaaccaattactgataatgtagaagatcatacagacaccgtcacatacactagtgatagcattaatacagtaagtgcatcatctggagaatccacaacagacgagactccggaaccaattactgataaagaagaagatcatacagtcacagacactgtctcatacactacagtaagtacatcatctggaattgtcactactaaatcaaccaccgatgatgcggatctttatgatacgtacaatgataatgatacagtaccaccaactactgtaggcggtagtacaacctctattagcaattataaaaccaaggactttgtagaaatatttggtattaccgcattaattatattgtcggccgtggcaatattctgtattacatattatatatataataaacgttcacgtaaatacaaaacagagaacaaagtctag

SEQ ID NO: 32 - DNA, 942 bp, Artificial sequence
  synthetic sequence
atgacacgattaccaatacttttgttactaatatcattagtatacgctacaccttttcctcagacatctaaaaaaataggtgatgatgcaactctatcatgtaatcgaaataatacaaatgactacgttgttatgagtgcttggtataaggagcccaattccattattcttttagctgctaaaagtgacgtcttgtattttgataattataccaaggataaaatatcttacgactctccatacgatgatctagttacaactatcacaattaaatcattgactgctagagatgccggtacttatgtatgtgcattctttatgacatcgcctacaaatgacactgataaagtagattatgaagaatactccacagagttgattgtaaatacagatagtgaatcgactatagacataatactatctggatctacacattcaccggaaactagttctgagaaacctgattatatagataattctaattgctcgtcggtattcgaaatcgcgactccggaaccaattactgataatgtagaagatcatacagacaccgtcacatacactagtgatagcattaatacagtaagtgcatcatctggagaatccacaacagacgagactccggaaccaattactgataaagaagaagatcatacagtcacagacactgtctcatacactacagtaagtacatcatctggaattgtcactactaaatcaaccaccgatgatgcggatcttcatgatacgtacaatgatacagtaccatcaactactgtaggcggtagtacaacctctattagcaattataaaaccaaggactttgtagaaatatttggtattaccgcattaattatattgtcggccgtggcaattttctgtattacgtattatatatgtaataaacgttcacgtaaatacaaaacagagaacaaagtctag

SEQ ID NO: 33 - DNA, 948 bp, Artificial sequence
  synthetic sequence
atggcacgattaccaatacttttgttactaatatcattagtatactctacaccttctcctcagacatctaaaaaaataggtgatgatgcaactctatcatgtaatcgaaataatacaaatgactacgttgttatgagtgcttggtataaggagcccaattccattattcttttagctgctaaaagcgacgtcttgtattttgataattataccaaggataaaatatcttacgactctccatacgatgatctagttacaactatcacaattaaatcattgactgctagagatgccggtacttatgtatgtgcattctttatgacatcgcctacaaatgacactgataaagtagattatgaagaatactccacagagttgattgtaaatacagatagtgaatcgactatagacataatactatctggatctacacattcaccggaaactagttctgagaaacctgattatatagataattctaattgctcgtcggtattcgaaatcgcgactccggaaccaattactgataatgtagaagatcatacagacaccgtcacatacactagtgatagcattaatacagtaagtgcatcatctggagaatccacaacagacgagactccggaaccaattactgataaagaagaagatcatacagtcacagacactgtctcatacactacagtaagtacatcatctggaattgtcactactaaatcaaccaccgatgatgcggatctttatgatacgtacaatgataatgatacagtaccatcaactactgtaggtagtagtacaacctctattagcaattataaaaccaaggactttgtagaaatatttggtattaccgcattaattatattgtcggccgtggcaatattctgtattacgtattatatatgtaataaacgttcacgtaaatacaaaacagagaacaaagtctag

SEQ ID NO: 34 - DNA, 945 bp, Artificial sequence
  synthetic sequence
atgacacgattaccaatacttttgttactaatatcattagtatacgctacaccttttcctcagacatctaaaaaaataggtgatgatgcaactctatcatgtaatcgaaataatacaaatgactacgttgttatgagtgcttggtataaggagcccaattccattattcttttagctgctaaaagcgacgtcttgtattttgataattataccaaggataaaatatcttacgactctccatacgatgatctagttacaactatcacaattaaatcattgactgctagagatgccggtacttatgtatgtgcattctttatgacatcaactacaaatgacactgataaagtagattatgaagaatactccacagagttgattgtaaatacagatagtgaatcgactatagacataatactatctggatctacacattcaccggaaactagttctaagaaacctgattatatagataattctaattgctcgtcggtattcgaaatcgcgactccggaaccaattactgataatgtagaagatcatacagacaccgtcacatacactagtgatagcattaatacagtaagtgcatcatctggagaatccacaacagacgagactccggaaccaattactgataaagaagatcatacagttacagacactgtctcatacactacagtaagtacatcatctggaattgtcactactaaatcaaccaccgatgatgcggatctttatgatacgtacaatgataatgatacagtaccaccaactactgtaggcggtagtacaacctctattagcaattataaaaccaaggactttgtagaaatatttggtattaccgcattaattatattgtcggccgtggcaattttctgtattacatattatatatataataaacgttcacgtaaatacaaaacagagaacaaagtctag

SEQ ID NO: 35 - DNA, 930 bp, Artificial sequence
  synthetic sequence
atgacacgattaccaatacttttgttactaatatcattagtatacgctacaccttttcctcagacatctaaaaaaataggtgatgatgcaactctatcatgtaatcgaaataatacaaatgactacgttgttatgagtgcttggtataaggagcccaattctattattcttttagctgctaaaagcgacgtcttgtattttgataattataccaaggataaaatatcttacgactctccatacgatgatctagttacaactatcacaattaaatcattgactgctagagatgccggtacttatgtatgtgcattctttatgacatcgcctacaaatgacactgataaagtagattatgaagaatactccacagagttgattgtaaatacagatagtgaatcgactatagacataatactatctggatctacacattcaccggaaactagttctgagaaacctgattatatagataattctaattgctcgtcggtattcgaaatcgcgactccggaaccaattactgataatgtagaagatcatacagacaccgtcacatacactagtgatagcattaatacagtaagtgcaacatctggagaatccacaacagacgagactccggaaccaattactgataaagaagatcatacagttacagacactgtctcatacactacagtaagtacatcatctggaattgtcactactaaatcaaccaccgatgatacgtacaatgataatgatacagtaccaccaactactgtaggcggtagtacaacctctattagcaattataaaaccaaggactttgtagaaatatttggtattaccgcattaattatattgtcggccgtggcaatattctgtattacgtattatatatgtaataaacgttcacgtaaatacaaaacagagaacaaagtctag

SEQ ID NO: 36 - protein, 315 aa, Artificial sequence
  synthetic sequence
MTRLPILLLLISLVYATPFPQTSKKIGDDATLSCNRNNTNDYVVMSAWYKEPNSIILLAAKSDVLYFDNYTKDKISYDSPYDDLVTTITIKSLTARDAGTYVCAFFMTSPTNDTDKVDYEEYSTELIVNTDSESTIDIILSGSTHSPETSSEKPDYIDNSNCSSVFEIATPEPITDNVEDHTDTVTYTSDSINTVSATSGESTTDETPEPITDKEEDHTVTDTVSYTTVSTSSGIVTTKSTTDDADLYDTYNDNDTVPSTTVGSSTTSISNYKTKDFVEIFGITALIILSAVAIFCITYYICNKRSRKYKTENKV

SEQ ID NO: 37 - protein, 315 aa, Artificial sequence
  synthetic sequence
MTRLPILLLLISLVYATPFPQTSKKIGDDATLSCNRNNTNDYVVMSAWYKEPNSIILLAAKSDVLYFDNYTKDKISYDSPYDDLVTTITIKSLTARDAGTYVCAFFMTSPTNDTDKVDYEEYSTELIVNTDSESTIDIILSGSTHSPETSSEKPDYIDNSNCSSVFEIATPEPITDNVEDHTDTVTYTSDSINTVSASSGESTTDETPEPITDKEEDHTVTDTVSYTTVSTSSGIVTTKSTTDDADLYDTYNDNDTVPSTTVGGSTTSISNYKTKDFVEIFGITALIILSAVAIFCITYYIYNKRSRKYKTENKV

SEQ ID NO: 38 - protein, 315 aa, Artificial sequence
  synthetic sequence
MTRLPILLLLISLVYATPFPQTSKKIGDDATLSCNRNNTNDYVVMSAWYKEPNSIILLAAKSDVLYFDNYTKDKISYDSPYDDLVTTITIKSLTARDAGTYVCAFFMTSTTNDTDKVDYEEYSTELIVNTDSESTIDIILSGSTHSPETSSEKPEDIDNFNCSSVFEIATPEPITDNVEDHTDTVTYTSDSINTVSASSGESTTDETPEPITDKEEDHTVTDTVSYTTVSTSSGIVTTKSTTDDADLYDTYNDNDTVPPTTVGGSTTSISNYKTKDFVEIFGITALIILSAVAIFCITYYIYNKRSRKYKTENKV

SEQ ID NO: 39 - protein, 313 aa, Artificial sequence
  synthetic sequence
MTRLPILLLLISLVYATPFPQTSKKIGDDATLSCNRNNTNDYVVMSAWYKEPNSIILLAAKSDVLYFDNYTKDKISYDSPYDDLVTTITIKSLTARDAGTYVCAFFMTSPTNDTDKVDYEEYSTELIVNTDSESTIDIILSGSTHSPETSSEKPDYIDNSNCSSVFEIATPEPITDNVEDHTDTVTYTSDSINTVSASSGESTTDETPEPITDKEEDHTVTDTVSYTTVSTSSGIVTTKSTTDDADLHDTYNDTVPSTTVGGSTTSISNYKTKDFVEIFGITALIILSAVAIFCITYYICNKRSRKYKTENKV

SEQ ID NO: 40 - protein, 315 aa, Artificial sequence
  synthetic sequence
MARLPILLLLISLVYSTPSPQTSKKIGDDATLSCNRNNTNDYVVMSAWYKEPNSIILLAAKSDVLYFDNYTKDKISYDSPYDDLVTTITIKSLTARDAGTYVCAFFMTSPTNDTDKVDYEEYSTELIVNTDSESTIDIILSGSTHSPETSSEKPDYIDNSNCSSVFEIATPEPITDNVEDHTDTVTYTSDSINTVSASSGESTTDETPEPITDKEEDHTVTDTVSYTTVSTSSGIVTTKSTTDDADLYDTYNDNDTVPSTTVGSSTTSISNYKTKDFVEIFGITALIILSAVAIFCITYYICNKRSRKYKTENKV

SEQ ID NO: 41 - protein, 314 aa, Artificial sequence
  synthetic sequence
MTRLPILLLLISLVYATPFPQTSKKIGDDATLSCNRNNTNDYVVMSAWYKEPNSIILLAAKSDVLYFDNYTKDKISYDSPYDDLVTTITIKSLTARDAGTYVCAFFMTSTTNDTDKVDYEEYSTELIVNTDSESTIDIILSGSTHSPETSSKKPDYIDNSNCSSVFEIATPEPITDNVEDHTDTVTYTSDSINTVSASSGESTTDETPEPITDKEDHTVTDTVSYTTVSTSSGIVTTKSTTDDADLYDTYNDNDTVPPTTVGGSTTSISNYKTKDFVEIFGITALIILSAVAIFCITYYIYNKRSRKYKTENKV

SEQ ID NO: 42 - protein, 309 aa, Artificial sequence
  synthetic sequence
MTRLPILLLLISLVYATPFPQTSKKIGDDATLSCNRNNTNDYVVMSAWYKEPNSIILLAAKSDVLYFDNYTKDKISYDSPYDDLVTTITIKSLTARDAGTYVCAFFMTSPTNDTDKVDYEEYSTELIVNTDSESTIDIILSGSTHSPETSSEKPDYIDNSNCSSVFEIATPEPITDNVEDHTDTVTYTSDSINTVSATSGESTTDETPEPITDKEDHTVTDTVSYTTVSTSSGIVTTKSTTDDTYNDNDTVPPTTVGGSTTSISNYKTKDFVEIFGITALIILSAVAIFCITYYICNKRSRKYKTENKV

SEQ ID NO: 43 - DNA, 954 bp, Artificial sequence
  synthetic sequence
atgaaaacgatttccgttgttacgttgttatgcgtactacctgctgttgtttattcaacatgtactgtacccactatgaataacgctaaattaacgtctaccgaaacatcgtttaataataaccagaaagttacgtttacatgtgatcagggatatcattcttcggatccaaatgctgtctgcgaaacagataaatggaaatacgaaaatccatgcaaaaaaatgtgcacagtttctgattacatctctgaactatataataaaccgctatacgaagtgaattccaccatgacactaagttgcaacggcgaaacaaaatattttcgttgcgaagaaaaaaatggaaatacttcttggaatgatactgttacgtgtcctaatgcggaatgtcaacctcttcaattagaacacggatcgtgtcaaccagttaaagaaaaatactcatttggggaatatatgactatcaactgtgatgttggatatgaggttattggtgcttcgtacataagttgtacagctaattcttggaatgttattccatcatgtcaacaaaaatgtgatataccgtctctatctaatggattaatttccggatctacattttctatcggtggcgttatacatcttagttgtaaaagtggttttatactaacgggatctccatcatccacatgtatcgacggtaaatggaatcccgtactcccaatatgtgtacgaactaacgaagaatttgatccagtggatgatggtcccgacgatgagacagatttgagcaaactctcgaaagacgttgtacaatatgaacaagaaatagaatcgttagaagcaacttatcatataatcatagtggcgttaacaattatgggcgtcatatttttaatctccgttatagtattagtttgttcctgtgacaaaaataatgaccaatataagttccataaattgctaccgtaa

SEQ ID NO: 44 - DNA, 954 bp, Artificial sequence
  synthetic sequence
atgaaaacgatttccgttgttacgttgttatgcgtactacctgctgttgtttattcaacatgtactgtacccactatgaataacgctaaattaacgtctaccgaaacatcgtttaataataaccagaaagttacgtttacatgtgatcagggatatcattcttcggatccaaatgctgtctgcgaaacagataaatggaaatacgaaaatccatgcaaaaaaatgtgcacagtttctgattacatctctgaactatataataaaccgctatacgaagtgaattccaccatgacactaagttgcaacggcgaaacaaaatattttcgttgcgaagaaaaaaatggaaatacttcttggaatgatactgttacgtgtcctaatgcggaatgtcaacctcttcaattagaacacggatcgtgtcaaccagttaaagaaaaatactcatttggggaatatataactatcaactgtgatgttggatatgaggttattggtgcttcgtacataagttgtacagctaattcttggaatgttattccatcatgtcaacaaaaatgtgatataccgtctctatctaatggattaatttccggatctacattttctatcggtggcgttatacatcttagttgtaaaagtggttttatactaacgggatctccatcatccacatgtatcgacggtaaatggaatcccatactcccaacatgtgtacgatctaacgaaaaatttgatccagtggatgatggtcccgacgatgagacagatttgagcaaactctcgaaagacgttgtacaatatgaacaagaaatagaatcgttagaagcaacttatcatataatcatagtggcgttaacaattatgggcgtcatatttttaatctccgttatagtattagtttgttcctgtgacaaaaataatgaccaatataagttccataaattgctaccgtga

SEQ ID NO: 45 - DNA, 954 bp, Artificial sequence
  synthetic sequence
atgaaaacgatttccgttgttacgttgttatgcgtactacctgctgttgtttattcaacatgtactgtacccactatgaataacgctaaattaacgtctaccgaaacatcgtttaatgataaacagaaagttacatttacatgtgatcagggatatcattctttggatccaaatgctgtctgcgaaacagataaatggaaatacgaaaatccatgcaagaaaatgtgcacagtttctgattatgtctctgaattatatgataagccattatacgaagtgaattccaccatgacactaagttgcaacggcgaaacaaaatattttcgttgcgaagaaaaaaatggaaatacttcttggaatgatactgttacgtgtcctaatgcggaatgtcaacctcttcaattagaacacggatcgtgtcaaccagttaaagaaaaatactcatttggggaatatatgactatcaactgtgatgttggatatgaggttattggtgcttcgtacataagttgtacagctaattcttggaatgttattccatcatgtcaacaaaaatgtgatataccgtctctatctaatggattaatttccggatctacattttctatcggtggcgttatacatcttagttgtaaaagtggttttatactaacgggatctccatcatccacatgtatcgacggtaaatggaatcccatactcccaacatgtgtacgatctaacgaaaaatttgatccagtggatgatggtcccgacgatgagacagatttgagcaaactctcgaaagacgttgtacaatatgaacaagaaatagaatcgttagaagcaacttatcatataatcatagtggcgttaacaattatgggcgtcatatttttaatctccgttatagtattagtttgttcctgtgacaaaaataatgaccaatataagttccataaattgctaccgtaa

SEQ ID NO: 46 - DNA, 942 bp, Artificial sequence; identical to SEQ ID NO: 32
  synthetic sequence

SEQ ID NO: 47 - DNA, 954 bp, Artificial sequence
  synthetic sequence
atgaaaacgatttccgttgttacgttgttatgcgtactacctgctgttgtttattcaacatgtactgtacccactatgaataacgctaaattaacgtctaccgaaacatcgtttaatgataaacagaaagttacatttacatgtgatcagggatatcattcttcggatccaaatgctgtctgtgaaacagataaatggaaatacgaaaatccatgcaaaaaaatgtgcacagtttctgattacatctctgaactatataataaaccgctatacgaagtgaattccaccatgacactaagttgcaacggcgaaacaaaatattttcgttgcgaagaaaaaaatggaaatacttcttggaatgatactgttacgtgtcctaatgcggaatgtcaacctcttcaattagaacacggatcgtgtcaaccagttaaagaaaaatactcatttggggaatatatgactatcaactgtgatgttggatatgaggttattggtgcttcgtacataagttgtacagctaattcttggaatgttattccatcatgtcaacaaaaatgtgatataccgtctctatctaatggattaatttccggatctacattttctatcggtggcgttatacatcttagttgtaaaagtggttttatactaacgggatctccatcatccacatgtatcgacggtaaatggaatcccatactcccaacatgtgtacgatctaacgaaaaatttgatccagtggatgatggtcccgacgatgagacagatttgagcaaactctcgaaagacgttgtacaatatgaacaagaaatagaatcgttagaagcaacttatcatataatcatagtggcgttaacaattatgggcgtcatatttttaatctccgttatagtattagtttgttcctgtgacaaaaataatgaccaatataagttccataaattgctaccgtga

SEQ ID NO: 48 - DNA, 954 bp, Artificial sequence
  synthetic sequence
atgaaaacgatttccgttgttacgttgttatgcgtactacctgctgttgtttattcaacatgtactgtacccactatgaataacgctaaattaacgtctaccgaaacatcgtttaatgataaacagaaagttacgtttacatgtgatcagggatatcattcttcggatccaaatgctgtctgcgaaacagataaatggaaatacgaaaatccatgcaaaaaaatgtgcacagtttctgattacatctctgaattatataataaaccgctatacgaagtgaattccaccatgacactaagttgcaacggcgaaacaaaatattttcgttgcgaagaaaaaaatggaaatacttcttggaatgatactgttacgtgtcctaatgcggaatgtcaacctcttcaattagaacacggatcgtgtcaaccagttaaagaaaaatactcatttggggaatatatgactatcaactgtgatgttggatatgaggttattggtgcttcgtacataagttgtacagctaattcttggaatgttattccatcatgtcaacaaaaatgtgatatgccgtctctatctaatggattaatttccggatctacattttctatcggtggcgttatacatcttagttgtaaaagtggttttacactaacggggtctccatcatccacatgtatcgacggtaaatggaatcccgtactcccaatatgtgtacgaactaacgaagaatttgatccagtggatgatggtcccgacgatgagacagatttgagcaaactctcgaaagacgttgtacaatatgaacaagaaatagaatcgttagaagcaacttatcatataatcatagtggcgttaacaattatgggcgtcatatttttaatctccgttatagtattagtttgttcctgtgacaaaaataatgaccaatataagttccataaattgctaccgtaa

SEQ ID NO: 49 - DNA, 954 bp, Artificial sequence
  synthetic sequence
atgaaaacgatttccgttgttacgttgttatgcgtactacctgctgttgtttattcaacatgtactgtacccactatgaataacgctaaattaacgtctaccgaaacatcgtttaatgataaacagaaagttacatttacatgtgatcagggatatcattctttggatccaaatgctgtctgtgaaacagataaatggaaatacgaaaatccatgcaaaaaaatgtgcacagtttctgattacatctctgaactatataataaaccgctatacgaagtgaattccaccatgacactaagttgcaacggcgaaacaaaatattttcgttgcgaagaaaaaaatggaaatacttcttggaatgatactgttacgtgtcctaatgcggaatgtcaacctcttcaattagaacacggatcgtgtcaaccagttaaagaaaaatactcatttggggaatatataactatcaactgtgatgttggatatgaggttattggtgcttcgtacataagttgtacagctaattcttggaatgttattccatcatgtcaacaaaaatgtgatataccgtctctatctaatggattaatttccggatctacattttctatcggtggcgttatacatcttagttgtaaaagtggttttatactaacgggatctccatcatccacatgtatcgacggtaaatggaatcccatactcccaacatgtgtacgatctaacgaaaaatttgatccagtggatgatggtcccgacgatgagacagatttgagcaaactctcgaaagacgttgtacaatatgaacaagaaatagaatcgttagaagcaacttatcatataatcatagtggcgttaacaattatgggcgtcatatttttaatctccgttatagtattagtttgttcctgtgacaaaaataatgaccaatataagttccataaattgctaccgtaa

SEQ ID NO: 50 - protein, 317 aa, Artificial sequence
  synthetic sequence
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGKWNPVLPICVRTNEEFDPVDDGPDDETDLSKLSKDVVQYEQEIESLEATYHIIIVALTIMGVIFLISVIVLVCSCDKNNDQYKFHKLLP

SEQ ID NO: 51 - protein, 317 aa, Artificial sequence
  synthetic sequence
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYITINCDVGYEVIGASYISCTANSWNVIPSCQQKCDIPSLSNGLISGSTFSIGGVIHLSCKSGFILTGSPSSTCIDGKWNPILPTCVRSNEKFDPVDDGPDDETDLSKLSKDVVQYEQEIESLEATYHIIIVALTIMGVIFLISVIVLVCSCDKNNDQYKFHKLLP

SEQ ID NO: 52 - protein, 317 aa, Artificial sequence
  synthetic sequence
MKTISVVTLLCVLPAVVYSTCTVPTMNNAKLTSTETSFNDKQKVTFTCDQGYHSLDPNAVCETDKWKYENPCKKMCTVSDYVSELYDKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGKWNPILPTCVRSNEKFDPVDDGPDDETDLSKLSKDVVQYEQEIESLEATYHIIIVALTIMGVIFLISVIVLVCSCDKNNDQYKFHKLLP

SEQ ID NO: 53 - protein, 317 aa, Artificial sequence
  synthetic sequence
MKTISVVTLLCVLPAVVYSTCTVPTMNNAKLTSTETSFNDKQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDMPSLSNGLISGSTFSIGGVIHLSCKSGFTLTGSPSSTCIDGKWNPVLPTCVRTNEKFDPVDDGPDDETDLSKLSKDVVQYEQEIESLEATYHIIIVALTIMGVIFLISVIVLVCSCDKNNDQYKFHKLLP

SEQ ID NO: 54 - protein, 317 aa, Artificial sequence
  synthetic sequence
MKTISVVTLLCVLPAVVYSTCTVPTMNNAKLTSTETSFNDKQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGKWNPILPTCVRSNEKFDPVDDGPDDETDLSKLSKDVVQYEQEIESLEATYHIIIVALTIMGVIFLISVIVLVCSCDKNNDQYKFHKLLP

SEQ ID NO: 55 - protein, 317 aa, Artificial sequence
  synthetic sequence
MKTISVVTLLCVLPAVVYSTCTVPTMNNAKLTSTETSFNDKQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDMPSLSNGLISGSTFSIGGVIHLSCKSGFTLTGSPSSTCIDGKWNPVLPICVRTNEEFDPVDDGPDDETDLSKLSKDVVQYEQEIESLEATYHIIIVALTIMGVIFLISVIVLVCSCDKNNDQYKFHKLLP

SEQ ID NO: 56 - protein, 317 aa, Artificial sequence
  synthetic sequence
MKTISVVTLLCVLPAVVYSTCTVPTMNNAKLTSTETSFNDKQKVTFTCDQGYHSLDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYITINCDVGYEVIGASYISCTANSWNVIPSCQQKCDIPSLSNGLISGSTFSIGGVIHLSCKSGFILTGSPSSTCIDGKWNPILPTCVRSNEKFDPVDDGPDDETDLSKLSKDVVQYEQEIESLEATYHIIIVALTIMGVIFLISVIVLVCSCDKNNDQYKFHKLLP

SEQ ID NO: 57 - protein, 185 aa, Artificial sequence
  synthetic sequence
MMTPENDEEQTSVFSATVYGDKIQGKNKRKRVIGLCIRISMVISLLSMITMSAFLIVRLNQCRSANEAAITDAAVAVAAASSTHRKVASSTTQYDHKESCNGLYYQGSCYILHSDYQLFSDAKANCTAESSTLPNKSDVLITWLIDYVEDTWGSDGNPITKTTSDYQDSDVSQEVRKYFCVKTMN

SEQ ID NO: 58 - protein, 185 aa, Artificial sequence
  synthetic sequence
MMTPENDEEQTSVFSATVYGDKIQGKNKRKRVIGLCIRISMVISLLSMITMSAFLIVRLNQCMSANEAAITDAAVAVAAASSTHRKVDSSTTQYDHKESCNGLYYQGSCYILHSDYQLFSDAKANCTAESSTLPNKSDVLITWLIDYVEDTWGSDGNPITKTTSDYQDSDVSQEVRKYFCVKTMN

SEQ ID NO: 59 - protein, 185 aa, Artificial sequence
  synthetic sequence
MMTPENDEEQTSVFSATVYGDKIQGKNKRKRVIGLCIRISMVISLLSMITMSAFLIVRLNQCMSANEAAITDAAVAVAAASSTHRKVASSTTQYDHKESCNGLYYQGSCYILHSDYQLFSDAKANCTAMSSTLPNKSDVLITWLIDYVEDTWGSDGNPITKTTSDYQDSDVSQEVRKYFCVKTMN

SEQ ID NO: 60 - protein, 185 aa, Artificial sequence
  synthetic sequence
MMTPENDEEQTSVFSATVYGDKIQGKNKRKRVIGLCIRISMVISLLSMITMSAFLIVRLNQCMSANEAAITDAAVAVAAASSTHRKVDSSTTQYDHKESCNGLYYQGSCYILHSDYQLFSDAKANCTAMSSTLPNKSDVLITWLIDYVEDTWGSDGNPITKTTSDYQDSDVSQEVRKYFCVKTMN

SEQ ID NO: 61 - protein, 168 aa, Artificial sequence
  synthetic sequence
MKSLNRQTVSMFKKLSVPAAIMMILSTIISGIGTFLHYKEELMPSACANGWIQYDKHCYLDTNIKTSTDNAVYQCRKLRARLPRPDTRHLRVLFSIFYKDYWVSLKKTNNKWLDINNDKDIDISKLTNFKQLNSTTDAEACYIYKSGKLVKTVCKSTQSVLCVKKFYK

SEQ ID NO: 62 - protein, 168 aa, Artificial sequence
  synthetic sequence
MKSLNRQTVSMFKKLSVPAAIMMILSTIISGIGTFLHYKEELMPSACANGWIQYDKHCYLDTNIKMSTDNAVYQCRKLRARLPRPDTRHLRVLHSIFYKDYWVSLKKTNNKWLDINNDKDIDISKLTNFKQLNSTTDAEACYIYKSGKLVKTVCKSTQSVLCVKKFYK

SEQ ID NO: 63 - protein, 168 aa, Artificial sequence
  synthetic sequence
MKSLNRQTVSMFKKLSVPAAIMMILSTIISGIGTFLHYKEELMPSACANGWIQYDKHCYLDTNIKMSTDNAVYQCRKLRARLPRPDTRHLRVLFSIFYKDYWVSLKKTNNKWLDINNDKDIDISKLTNFKQLNSTTDAEACYIYKSGKLVETVCKSTQSVLCVKKFYK

SEQ ID NO: 64 - protein, 168 aa, Artificial sequence
  synthetic sequence
MKSLNRQTVSMFKKLSVPAAIMMILSTIISGIGTFLHYKEELMPSACANGWIQYDKHCYLDTNIKMSTDNAVYQCRKLRARLPRPDTRHLRVLHSIFYKDYWVSLKKTNNKWLDINNDKDIDISKLTNFKQLNSTTDAEACYIYKSGKLVETVCKSTQSVLCVKKFYK

SEQ ID NO: 65 - protein, 168 aa, Artificial sequence
  synthetic sequence
MKSLNRQTVSMFKKLSVPAAIMMILSTIISGIGTFLHYKEELMPSACANGWIQYDKHCYLDTNIKMSTDNAVYQCRKLRARLPGPDTRHLRVLFSIFYKDYWVSLKKTNNKWLDINNDKDIDISKLTNFKQLNSTTDAEACYIYKSGKLVKTVCKSTQSVLCVKKFYK

SEQ ID NO: 66 - protein, 168 aa, Artificial sequence
  synthetic sequence
MKSLNRQTVSMFKKLSVPAAIMMILSTIISGIGTFLHYKEELMPSACANGWIQYDKHCYLDTNIKMSTDNAVYQCRKLRARLPRPDTRHLSVLFSIFYKDYWVSLKKTNNKWLDINNDKDIDISKLTNFKQLNSTTDAEACYIYKSGKLVKTVCKSTQSVLCVKKFYK

SEQ ID NO: 67 - protein, 168 aa, Artificial sequence
  synthetic sequence
MKSLNRQTVSMFKKLSVPAAIMMILSTIISGIGTFLHYKEELMPSACANGWIQYDKHCYLDTNIKMSTDNAVYQCRKLRARLPRPDTRHLRVLFSIFYKDYWVSLKKTNNKWLDINNDKDIDISKLENFKQLNSTTDAEACYIYKSGKLVKTVCKSTQSVLCVKKFYK

SEQ ID NO: 68 - protein, 317 aa, Artificial sequence
  synthetic sequence
MKTISVVTLLCVLPAVVYSTCTVPTMNNAKLTSTETSFNNNQKVTFTCDQGYHSSDPNAVCETDKWKYENPCKKMCTVSDYISELYNKPLYEVTSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGKWNPVLPICVRTNEEFDPVDDGPDDETDLSKLSKDVVQYEQEIESLEATYHIIIVALTIMGVIFLISVIVLVCSCDKNNDQYKFHKLLP

SEQ ID NO: 69 - protein, 317 aa, Artificial sequence
  synthetic sequence
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGMTFSIGGVIHLSCKSGFILTGSPSSTCIDGKWNPVLPICVRTNEEFDPVDDGPDDETDLSKLSKDVVQYEQEIESLEATYHIIIVALTIMGVIFLISVIVLVCSCDKNNDQYKFHKLLP

SEQ ID NO: 70 - protein, 317 aa, Artificial sequence
  synthetic sequence
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGKWNPVLPICVRTNEEFDPVDDGPDDETDLSKLSKDVVQYEQEIEILEATYHIIIVALTIMGVIFLISVIVLVCSCDKNNDQYKFHKLLP

SEQ ID NO: 71 - protein, 317 aa, Artificial sequence
  synthetic sequence
MKTISVVTLLCVLPAVVYSTCTVPTMNNAKLTSTETSFGNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGKWNPVLPICVRTNEEFDPVDDGPDDETDLSKLSKDVVQYEQEIEILEATYHIIIVALTIMGVIFLISVIVLVCSCDKNNDQYKFHKLLP

SEQ ID NO: 72 - protein, 317 aa, Artificial sequence
  synthetic sequence
MKTISVVTLLCVLPAVVYSTCTVPTMNNAKLTSTETSFNNNQKVTFTCDQGYHSSDPNAVCETDKWKYENPCKKMCTVSDYISELYNKPRYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGKWNPVLPICVRTNEEFDPVDDGPDDETDLSKLSKDVVQYEQEIEVLEATYHIIIVALTIMGVIFLISVIVLVCSCDKNNDQYKFHKLLP

SEQ ID NO: 73 - protein, 317 aa, Artificial sequence
  synthetic sequence
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGCWNPVLPICVRTNEEFDPVDDGPDDETDLSKLSKDVVQYEQEIELLEATYHIIIVALTIMGVIFLISVIVLVCSCDKNNDQYKFHKLLP

SEQ ID NO: 74 - protein, 317 aa, Artificial sequence
  synthetic sequence
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGKWNPDLPLCWRYNERFDPVDDGPDDETDLSKLSKDVVQYEQEIESLEATYHIIIVALTIMGVIFLISVIVLVCSCDKNNDQYKFHKLLP

SEQ ID NO: 75 - protein, 317 aa, Artificial sequence
  synthetic sequence
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGKWNPVLPPCRRRNEGFDPVDDGPDDETDLSKLSKDVVQYEQEIESLEATYHIIIVALTIMGVIFLISVIVLVCSCDKNNDQYKFHKLLP

SEQ ID NO: 76 - protein, 317 aa, Artificial sequence
  synthetic sequence
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGKWNPVLPICVRTTEVFDPSDDRPDDETDLSKLSKDVVQYEQEIESLEFTYHIIIVALTIMGVIFLISVIVLVCSCDKNNDQYKFHKLLP

SEQ ID NO: 77 - protein, 317 aa, Artificial sequence
  synthetic sequence
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGKWNPVLPICVRTGESFDPWYDAPDDETDLSKLSKDVVQYEQEIESLEFTYHIIIVALTIMGVIFLISVIVLVCSCDKNNDQYKFHKLLP

SEQ ID NO: 78 - protein, 317 aa, Artificial sequence
  synthetic sequence
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGKWNPVLPICVRTNEEFDPVDDGPDDETDLSKLSKAVVQYEQSIGSLFATYHIIIVALTIMGVIFLISVIVLVCSCDKNNDQYKFHKLLP

SEQ ID NO: 79 - protein, 317 aa, Artificial sequence
  synthetic sequence
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGKWNPVLPICVRTNEEFDPVDDGPDDETDLSKLSKVVVQYTQGIPSLSATYHIIIVALTIMGVIFLISVIVLVCSCDKNNDQYKFHKLLP

SEQ ID NO: 80 - protein, 315 aa, Artificial sequence
  synthetic sequence
MTRLPILLLLISLVYATPFPQTSKKIGDDATLSCNRNNTNDYVVMSAWYKEPNSIILLAAKSDVLYFDNYTKDKISYDSPYDDLVTTITIKSLTARDAGTYVCAFFMTSPTNDTDKVDYEEYSTELIVNTDSESTIDIILSGSTHSPETSSEKPDYIDNSNCSSVFEIATPEPITDNVEDHTDTVTYTSDSINTVSATSGESTTDETPEPITDKEEDHTVTDTVSYTTVSTSSGIVTTKSTTDDADLYDTYNDNDTVPSTTVGSSTTSFSNYKTKDFVEIFGITALIILSAVAIFCITYYICNKRSRKYKTENKV

SEQ ID NO: 81 - protein, 168 aa, Artificial sequence
  synthetic sequence
MKSLNRQTVSMFKKLSVPAAIMMILSTIISGIGTFLHYKEELMPSACANGWIQYDKHCYLDTNIKMSTDNAVYQCRKLRARLPRPDTRHLAVLFSIFYKDYWVSLKKTNNKWLDINNDKDIDISKLTNFKQLNSTTDAEACYIYKSGKLVKTVCKSTQSVLCVKKFYK

SEQ ID NO: 82 - protein, 317 aa, Artificial sequence
  synthetic sequence
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLIFGSTFSIGGVIHLSCKSGFILTGSPSSTCIDGKWNPVLPICVRTNEEFDPVDDGPDDETDLSKLSKDVVQYEQEIESLEATYHIIIVALTIMGVIFLISVIVLVCSCDKNNDQYKFHKLLP

SEQ ID NO: 83 - protein, 317 aa, Artificial sequence
  synthetic sequence
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLIVGSTFSIGGVIHLSCKSGFILTGSPSSTCIDGKWNPVLPICVRTNEEFDPVDDGPDDETDLSKLSKDVVQYEQEIESLEATYHIIIVALTIMGVIFLISVIVLVCSCDKNNDQYKFHKLLP

(19) United States
(12) Patent Application Publication
(10) Pub. No.: US 2023/0002740 A1
(43) Pub. Date: Jan. 5, 2023

(54) VARIANT ONCOLYTIC VACCINIA VIRUS AND METHODS OF USE THEREOF

(71) Applicant: IGNITE IMMUNOTHERAPY, INC, New York, NY (US)

(72) Inventors: David H. Kirn, Mill Valley, CA (US); Liliana Maruri Avidal, Oakland, CA (US); Todd Brandon Slaby, Emeryville, CA (US); Darin Michael Abbadessa, San Diego, CA (US); Nathaniel Phillip Gulizia, La Mesa, CA (US); Melissa A. Kotterman, Oakland, CA (US)

(21) Appl. No.: 17/782,121

(22) PCT Filed: Dec. 9, 2020
(86) PCT No.: PCT/IB2020/061707

§ 371 Date: Jun. 2, 2022

Related U.S. Application Data
(60) Provisional application No. 62/947,204, filed on Dec. 12, 2019.
(60) Provisional application No. 62/947,202, filed on Dec. 12, 2019.
(60) Provisional application No. 62/947,200, filed on Dec. 12, 2019.

(51) Int. Cl. C12N 7/00; A61P 35/00; A61K 35/768

(52) U.S. Cl. CPC C12N 7/00; A61P 35/00; A61K 35/768; C12N 2710/24121

(57) ABSTRACT

The present disclosure provides a replication-competent, recombinant oncolytic vaccinia virus (OVV) comprising one of more of a) a nucleotide sequence encoding a variant A33 polypeptide, b) a nucleotide sequence encoding a variant A34 polypeptide, and c) a nucleotide sequence encoding a variant B5 polypeptide, wherein the variant A33, variant A34, and variant B5 polypeptides comprise one or more amino acid substitutions that provide for enhanced virus spreading or enhanced EEV production as compared with a virus encoding a corresponding wild-type A33, A 34, and B5 polypeptide. The present disclosure also provides compositions comprising the OVV and use of the OVV or the composition for inducing oncolysis in an individual having a tumor.

### CROSS-REFERENCE TO RELATED APPLICATIONS

This application claims the benefit of U.S. Provisional Application No. 62/947,200 filed Dec. 12, 2019, U.S. Provisional Application No. 62/947,202 filed Dec. 12, 2019, and U.S. Provisional Application No. 62/947,204 filed Dec. 12, 2019. The disclosure of each of the provisional applications is incorporated herein by reference in its entirety.

### INCORPORATION BY REFERENCE OF SEQUENCE LISTING PROVIDED AS A TEXT FILE

A Sequence Listing is provided herewith as a text file, “PC040318A_SeqListing_ST25.txt” created on Nov. 3, 2020 and having a size of 152 KB. The contents of the text file are incorporated by reference herein in their entirety.

### BACKGROUND

The present disclosure relates to oncolytic viruses in general and to recombinant oncolytic vaccinia virus with enhanced anti-tumor properties in particular.

Oncolytic viruses (OVs) are viruses that replicate selectively or more efficiently in cancer cells than in non-cancer cells. This group of viruses includes viruses found in nature (e.g., wild-type or native virus), as well as viruses that are engineered from a native virus by gene disruptions or gene additions so as to improve its anti-tumor properties, such as tumor selectivity or preferential replication in tumor cells, host tropism, surface attachment, lysis, and spread. Live replicating OVs have been tested in clinical trials in a variety of human cancers. OVs can induce anti-tumor immune responses, as well as direct lysis of tumor cells (i.e., oncolysis). Common OVs include attenuated strains of Herpes Simplex Virus (HSV), Adenovirus (Ad), Measles Virus (MV), Coxsackie virus (CV), Vesicular Stomatitis Virus (VSV), and Vaccinia Virus (VV).

Vaccinia virus, the prototypical member of the Orthopoxvirus genus, replicates in the cytoplasm of a host cell. During replication, three morphologically and antigenically distinct forms of the virus are produced: the intracellular mature virions (IMV), the intracellular enveloped virions (IEV), and extracellular virions. A subset of IMV, the first infectious progeny produced, are trafficked to the trans-Golgi network (TGN), where they are enveloped with two additional membranes to produce IEV. IEV are transported through the cytoplasm to the cell periphery, where the outermost membrane fuses with the plasma membrane to release a double membraned form, termed EV. EV that remain on the cell surface are called cell-associated enveloped virion (CEV), while EV that are no longer attached to the cell surface are called extracellular enveloped virion (EEV). IMV is the most abundant infectious form and is thought to be responsible for spread between hosts; the CEV is believed to play a role in cell-to-cell spread; and the EEV is thought to be important for long range dissemination within the host organism.

VV contains a double-stranded DNA genome of approximately 200 kbp that is predicted to encode more than 200 open reading frames. Seven proteins encoded by the virus, namely, A33, A34, A36, A56, B5, F12, and F13, are unique to enveloped forms (IEV/EEV/CEV) of the virus. Vaccinia virus open reading frames are designated by a capital letter indicating a HindIII restriction endonuclease fragment, a number indicating the position in the HindIII fragment, and a letter (L or R) indicating the direction of transcription, e.g., A34R. The corresponding protein is designated by a capital letter and number, e.g., A34. Specific to the extracellular virion membrane, glycoproteins A33, A34, and B5 are exposed on the surface of EV and have roles during extracellular virion formation and subsequent infection.

### SUMMARY

The present disclosure provides a replication-competent, recombinant oncolytic vaccinia virus (hereinafter referred to as “oncolytic vaccinia virus,” or “OVV”) that comprises a nucleotide sequence encoding a variant viral polypeptide, wherein the variant viral polypeptide provides for enhanced virus spreading or enhanced EEV production. The term “enhanced” as used herein means the same as, and is used interchangeably with, “increased” in the present disclosure. In some aspects, the OVV comprises a nucleotide sequence encoding a variant viral polypeptide selected from a variant A33 polypeptide, a nucleotide sequence encoding a variant A34 polypeptide, a nucleotide sequence encoding variant B5 polypeptide, or any combination of the nucleotide sequences above, where the variant A33, variant A34, and variant B5 polypeptides comprise 1, 2, 3, 4, 5, 6, or more amino acid mutations (such as substitutions, deletions, or insertions) that provide for enhanced virus spreading or enhanced EEV production, compared with a corresponding polypeptide without the respective mutations. In some embodiments, the OVV is constructed based on strain Copenhagen. In other embodiments, the OVV is constructed based on strain Western Reserve.

The present disclosure further provides compositions comprising the OVV. The present disclosure also provides methods of inducing oncolysis in an individual having a tumor, the methods comprising administering to the individual an effective amount of the OVV of the present disclosure or a composition of the present disclosure. The disclosure also provides an OVV or composition of the present disclosure for use in methods of inducing oncolysis in an individual having a tumor, and the use of an OVV or composition of the present disclosure in the manufacture of a medicament for use in such methods.

### BRIEF DESCRIPTION OF THE DRAWINGS

FIG. 1A-1C provide alignments of A33R nucleotide and A33 amino acid sequences for common strains of Vaccinia virus. A and B) A33R nucleotide sequences for Copenhagen (SEQ ID NO:1), Ankara (SEQ ID NO:2), IHD-J (SEQ ID NO:3), Lister (SEQ ID NO:4), Tian Tan (SEQ ID NO:5), Western Reserve (SEQ ID NO:6), and Wyeth (SEQ ID NO: 7) showing nucleotide locations between and across the genes. C) A33 amino acid sequences for Copenhagen (SEQ ID NO:8), Ankara (SEQ ID NO:9), IHD-J (SEQ ID NO:10), Lister (SEQ ID NO:11), Tian Tan (SEQ ID NO:12), Western Reserve (SEQ ID NO:13), and Wyeth (SEQ ID NO:14) showing amino acid locations between and across the proteins.

FIG. 2A-2C provide alignments of A34R nucleotide and A34 amino acid sequences for common strains of Vaccinia virus. A and B) A34R nucleotide sequences for Copenhagen (SEQ ID NO:15), Ankara (SEQ ID NO:16), IHD-J (SEQ ID NO:17), Lister (SEQ ID NO:18), Tian Tan (SEQ ID NO:19), Western Reserve (SEQ ID NO:20), and Wyeth (SEQ ID NO: 21) showing nucleotide locations between and across the genes. C) A34 amino acid sequences for Copenhagen (SEQ ID NO:22), Ankara (SEQ ID NO:23), IHD-J (SEQ ID NO:24), Lister (SEQ ID NO:25), Tian Tan (SEQ ID NO:26), Western Reserve (SEQ ID NO:27), and Wyeth (SEQ ID NO:28) showing amino acid locations between and across the proteins.

FIG. 3A-3D provide alignments of A56R nucleotide and A56 amino acid sequences for common strains of Vaccinia virus. A-C) A56R nucleotide sequences for Copenhagen (SEQ ID NO:29), Ankara (SEQ ID NO:30), IHD-J (SEQ ID NO:31), Lister (SEQ ID NO:32), Tian Tan (SEQ ID NO:33), Western Reserve (SEQ ID NO:34), and Wyeth (SEQ ID NO: 35) showing nucleotide locations between and across the genes. D) A56 amino acid sequences for Copenhagen (SEQ ID NO:36), Ankara (SEQ ID NO:37), IHD-J (SEQ ID NO:38), Lister (SEQ ID NO:39), Tian Tan (SEQ ID NO:40), Western Reserve (SEQ ID NO:41), and Wyeth (SEQ ID NO:42) showing amino acid locations between and across the proteins.

FIG. 4A-4D provide alignments of B5R nucleotide and B5 amino acid sequences for common strains of Vaccinia virus. A-C) B5R nucleotide sequences for Copenhagen (SEQ ID NO:43), Ankara (SEQ ID NO:44), IHD-J (SEQ ID NO:45), Lister (SEQ ID NO:46), Tian Tan (SEQ ID NO:47), Western Reserve (SEQ ID NO:48), and Wyeth (SEQ ID NO: 49) showing nucleotide locations between and across the genes. D) B5 amino acid sequences for Copenhagen (SEQ ID NO:50), Ankara (SEQ ID NO:51), IHD-J (SEQ ID NO:52), Lister (SEQ ID NO:53), Tian Tan (SEQ ID NO:54), Western Reserve (SEQ ID NO:55), and Wyeth (SEQ ID NO:56) showing amino acid locations between and across the proteins.

FIG. 5 provides a schematic example of a single stage of the directed evolution process used to identify EEV variants in vitro.

FIG. 6 provides data on the frequency of specific vaccinia virus variants in various rounds of the directed evolution process to identify variants capable of enhanced spreading and EEV production following infection of different human primary cancer cells and VEGF-stimulated endothelial cells.

FIG. 7A-7B provide data on virus spreading and EEV production of vaccinia virus variants containing A33 and A34 substitutions.

FIG. 8A-8D provide data on vaccinia virus production of infectious virus released to the supernatant early in the infection cycle (potentially EEVs) in representative human cancer cell lines.

FIG. 9A-9B provide data on vaccinia virus spreading in representative human cancer cell lines.

FIG. 10A-10B provide data for different variant vaccinia virus substitutions and combinations of substitutions in the absence of a K151E substitution in A34 on EEV production and viral spreading.

FIG. 11A-11B provide data for different variant vaccinia virus substitutions and combinations of substitutions in addition to the K151E substitution in A34 on EEV production and viral spreading.

FIG. 12A-12B provide data for different variants of vaccinia virus in production of physical EEVs and specific infectivity in HeLa S3 (cervical adenocarcinoma) cell line.

FIG. 13A-13B provide data for different variant vaccinia virus substitutions in combination with a K151E substitution in A34 on a Western Reserve strain on EEV production and viral spreading.

FIG. 14 provides data on the frequency of specific vaccinia virus variants in the final round of the directed evolution process to identify variants capable of enhanced virus spreading and EEV production following infection and selection on HCT-116 human colorectal cancer cells.

FIG. 15A-15F provide data on virus spreading and EEV production of additional vaccinia virus variants containing A34 and A56 substitutions.

FIG. 16A-16B provide alignments of A33 and A34 amino acid sequences for the vaccinia virus variants. A) A33 amino acid sequences for Copenhagen (SEQ ID NO:8), the M63R substitution (SEQ ID NO:57), the A88D substitution (SEQ ID NO:58), the E129M substitution (SEQ ID NO:59), and the A88D and E129M substitutions (SEQ ID NO:60) showing amino acid locations between and across the proteins. B) A34 amino acid sequences for Copenhagen (SEQ ID NO:22), the M66T substitution (SEQ ID NO:61), the F94H substitution (SEQ ID NO:62), the K151E substitution (SEQ ID NO:63), the F94H and K151E substitutions (SEQ ID NO:64), the R84G substitution (SEQ ID NO:65), the R91A substitution (SEQ ID NO:81), the R91S substitution (SEQ ID NO:66), and the T127E substitution (SEQ ID NO:67) showing amino acid locations between and across the proteins.

FIG. 17 provide data on the frequency of specific vaccinia virus in the final round of the directed evolution process to identify variants capable of enhanced virus spreading and EEV production following infection of Colo 205 human colorectal cancer cells with vaccinia virus libraries.

FIG. 18A-18B provide data on the frequency of specific vaccinia virus variants in the final round of the directed evolution process to identify variants capable of enhanced virus spreading and EEV production following infection of MDA-MB-231 human breast cancer cells in the absence or presence of serum from donors vaccinated with vaccinia virus.

FIG. 19A-19B provide data on virus spreading and EEV production of vaccinia virus variants containing B5 substitutions.

FIG. 20A-20D provide data on vaccinia virus infectious virions in the supernatant (potential EEVs) in representative human cancer cell lines.

FIG. 21A-21B provide data on the frequency of specific vaccinia virus variants in the directed evolution process to identify variants capable of enhanced spread in vivo.

FIG. 22A-22D provide data on virus spreading and EEV production of additional vaccinia virus variants containing A33/A34 or B5 substitutions.

FIG. 23 provides an alignment of B5 amino acid sequences for the vaccinia virus variants. B5 protein sequences for Copenhagen (SEQ ID NO:50), the N94T substitution (SEQ ID NO:68), the S197F substitution (SEQ ID NO:82), the S197V substitution (SEQ ID NO:83), the S199M substitution (SEQ ID NO:69), the S273I substitution (SEQ ID NO:70), the N39G and S273I substitutions (SEQ ID NO:71), the L90R and S273V substitutions (SEQ ID NO:72), the K229C and S273L substitutions (SEQ ID NO:73), the V233D, I236L, V238W, T240Y, and E243R substitutions (SEQ ID NO:74), the I236P, V238R, T240R, and E243G substitutions (SEQ ID NO:75), the N241T, E243V, V247S, G250R, and A276F substitutions (SEQ ID NO:76), the N241G, E243S, V247W, D248Y, G250A, and A276F substitutions (SEQ ID NO:77), the D263A, E270S, E272G, and E275F substitutions (SEQ ID NO:78), and the D263V, E268T, E270G, E272P, and E275S substitutions (SEQ ID NO:79) showing amino acid locations between and across the proteins.

FIG. 24 provides an alignment of A56 amino acid sequence for the vaccinia virus variant. A56 amino acid sequences for Copenhagen (SEQ ID NO:36) and the I269F substitution (SEQ ID NO:80) showing amino acid locations between and across the proteins.

### DETAILED DESCRIPTION

### Definitions

Before describing the present invention, several terms used in the context of the present disclosure will be defined. In addition to these terms, others are defined elsewhere in the specification, as necessary. Unless otherwise expressly defined herein, terms of art used in this specification will have their art-recognized meanings.

The term “oncolytic” vaccinia virus refers to a vaccinia virus that preferentially infects and kills cancer cells, compared to normal (non-cancerous) cells.

The term “heterologous” refers to a nucleotide or polypeptide sequence that is not found in the native nucleic acid or protein, respectively. For example, in the context of a recombinant vaccinia virus of the present disclosure, a nucleic acid comprising a nucleotide sequence encoding a “heterologous” immunomodulatory polypeptide is a nucleic acid that is not found naturally in vaccinia virus, i.e., the encoded immunomodulatory polypeptide is not encoded by naturally-occurring vaccinia virus.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc.

A “therapeutically effective amount” or “efficacious amount” refers to the amount of an agent (e.g., a replication-competent, recombinant oncolytic vaccinia virus of the present disclosure), or combined amounts of two agents (e.g., a replication-competent, recombinant oncolytic vaccinia virus of the present disclosure and a second therapeutic agent), that, when administered to a mammal or other subject for treating a disease, is sufficient to effect such treatment for the disease. The “therapeutically effective amount” will vary depending on the agent(s), the disease and its severity and the age, weight, etc., of the subject to be treated.

The term “variant” polypeptide refers to a polypeptide the amino acid sequence of which exhibits at least 90%, but less than 100%, identity with the amino acid sequence of a reference polypeptide; provided said variant has a biological activity as defined herein. The variant maybe arrived at by modification of the amino acid sequence of the reference polypeptide by such modifications as insertion, substitution, or deletion of one or more amino acids. Accordingly, the term “variant” polypeptide encompasses fragments of a reference polypeptide that comprises a sufficient number of contiguous amino acid residues to confer a desired biological property.

The term “substitution” refers to the replacement of one amino acid in a polypeptide with a different amino acid. In the context of the present disclosure, a substitution in a variant is indicated as: original amino acid-position-substituted amino acid. Accordingly, the notation “K151E” means, that the variant comprises a substitution of Lysine (K) with Glutamic acid (E) in the variant amino acid position corresponding to the amino acid in position 151 in the parent polypeptide.

Before the present invention is further described, it is to be understood that this invention is not limited to any particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a vaccinia virus” includes a plurality of such vaccinia viruses and reference to “the A33 polypeptide” includes reference to one or more A33 polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Oncolytic Vaccinia Virus

The present disclosure provides a replication-competent, recombinant oncolytic vaccinia virus (also referred to as “oncolytic vaccinia virus,” or “OVV”) that exhibits desirable oncolytic properties. It can derive from any vaccinia virus strain, preferably Elstree, Wyeth, Copenhagen and Western Reserve strains. Unless otherwise indicated, the gene nomenclature used herein is that of Copenhagen vaccinia strain. The OVV may be derived from a parent vaccinia virus by altering one or more viral gene(s) that changes the production, activity, function, or any other properties of the gene product (such as deletion, insertion, substitution of one or more nucleotides within the viral gene or its regulatory elements) and/or inserting one or more transgenes that encode exogenous polypeptides (i.e., polypeptides that are not naturally expressed by the virus).

In some aspects, the present disclosure provides an OVV that comprises a nucleotide sequence encoding a variant viral polypeptide, wherein the variant viral polypeptide provides for one or more enhanced oncolytic properties of the virus, such as enhanced virus spreading or enhanced EEV production. Effects of a variant viral polypeptide on virus spreading or EEV production can be determined using the methods described in the Examples provided in the specification, as well as any other suitable methods known in the art.

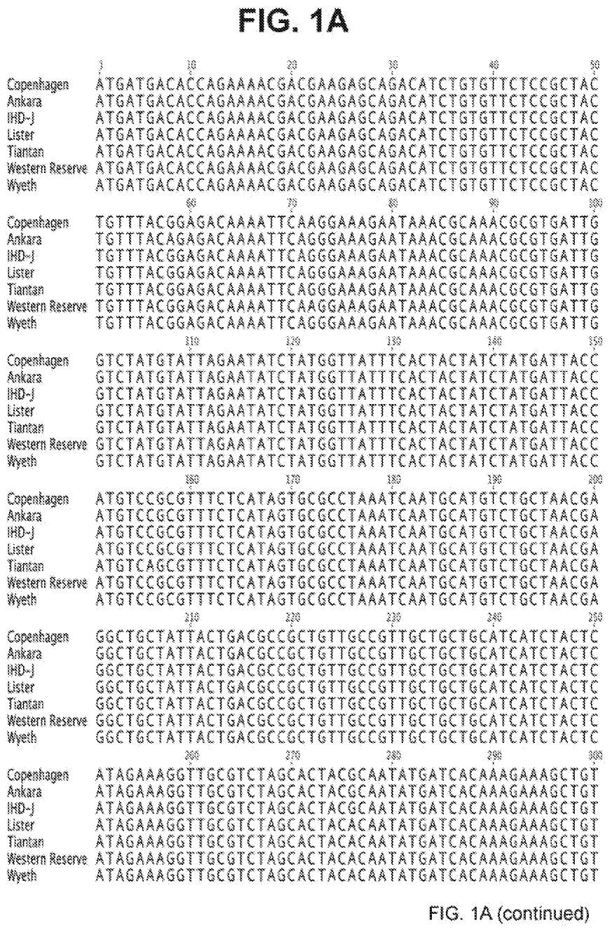

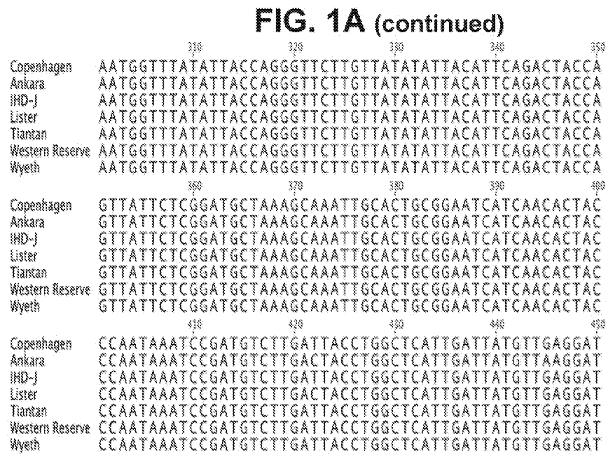

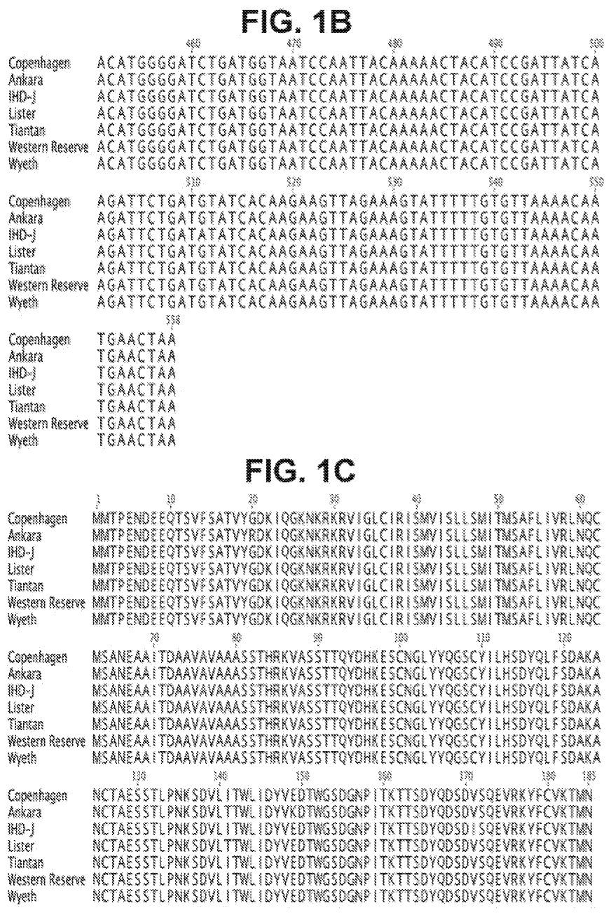

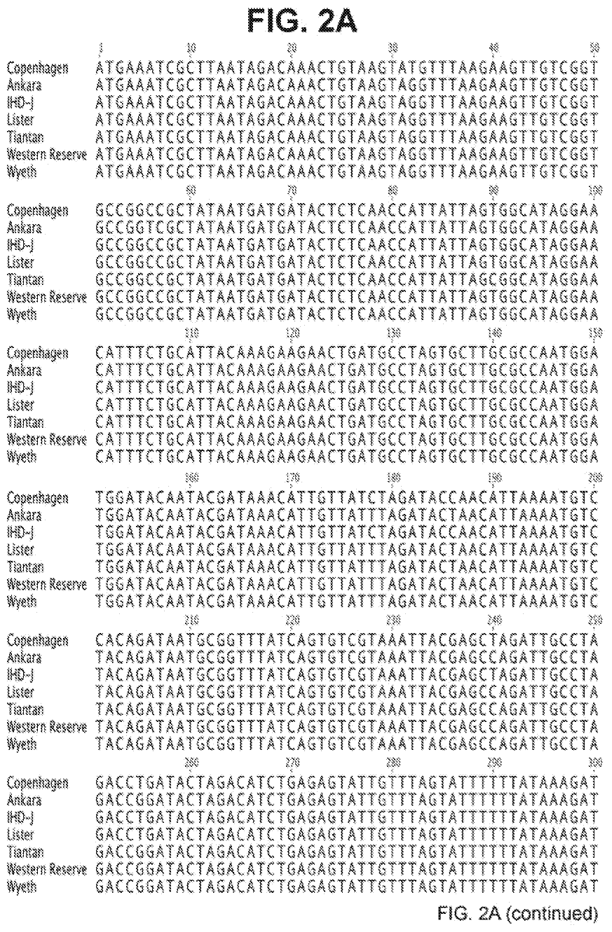

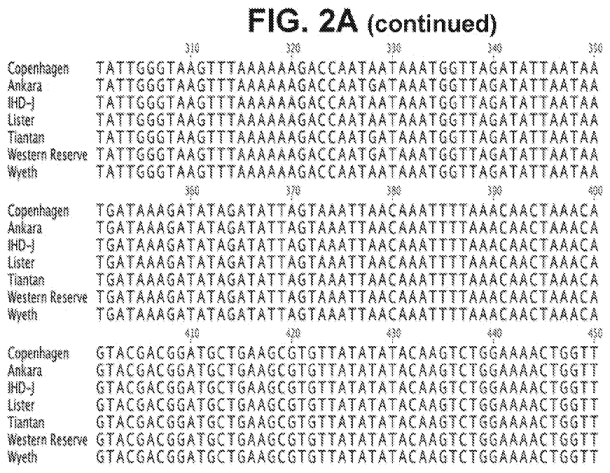

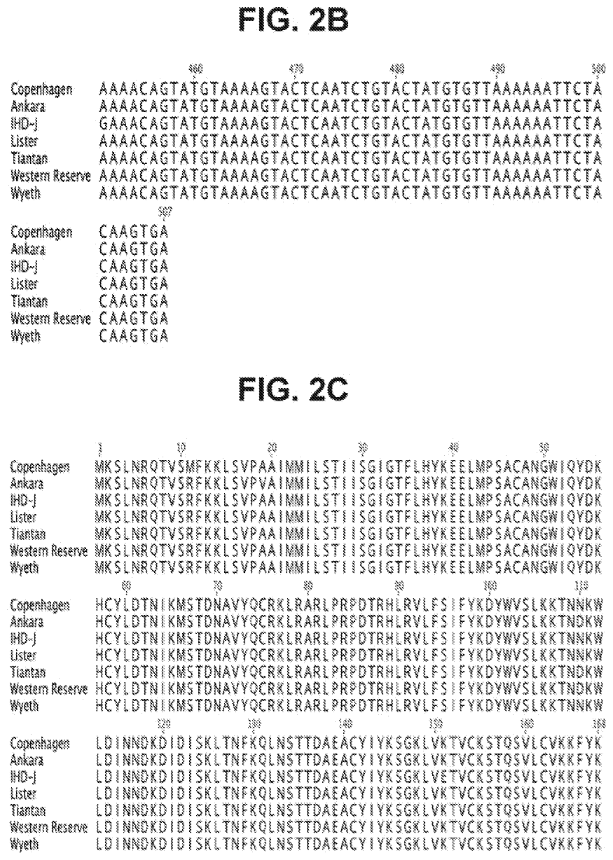

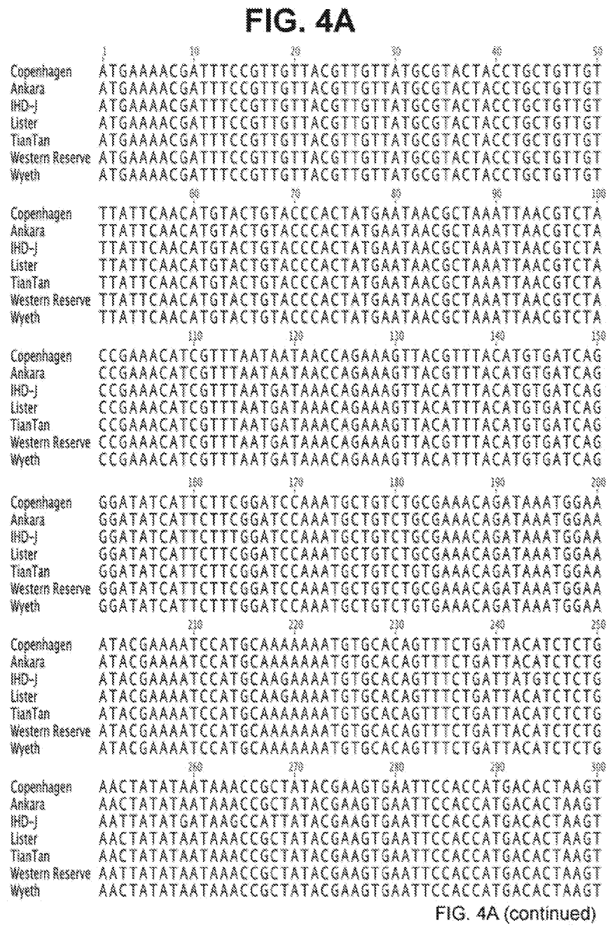

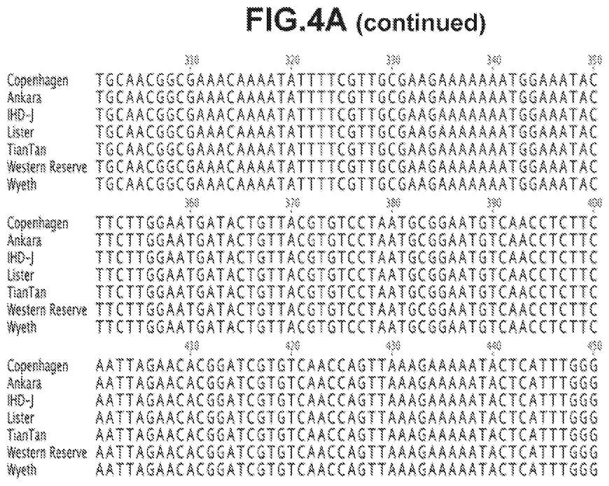

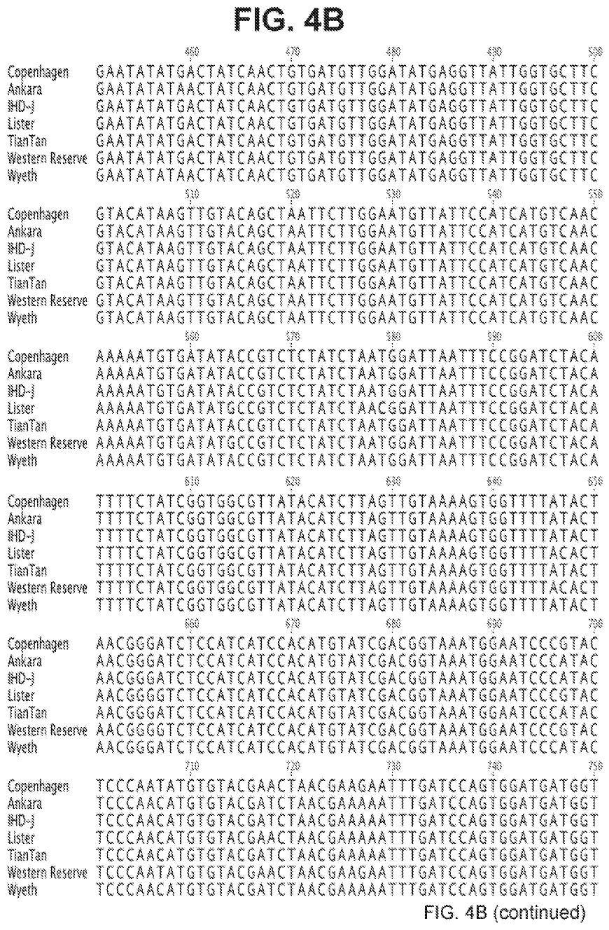

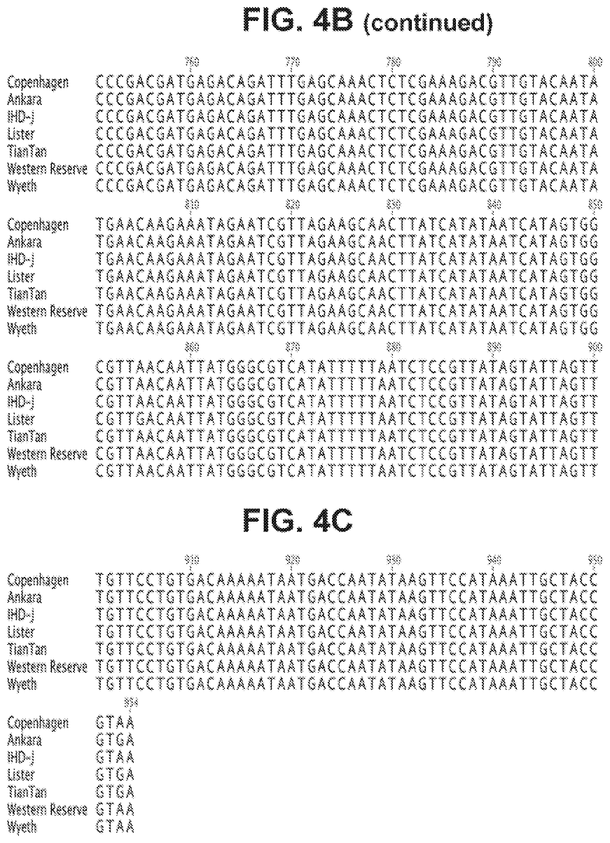

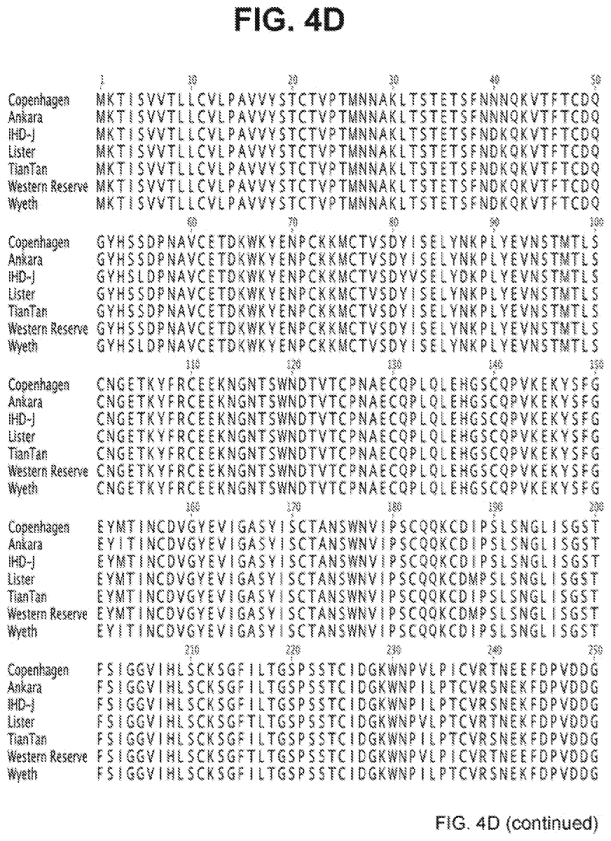

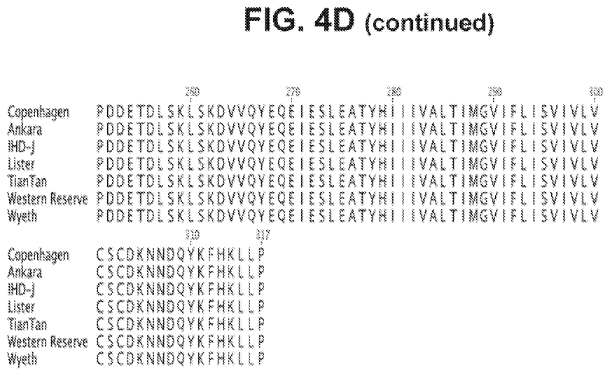

In some embodiments, the OVV comprises a nucleotide sequence encoding a variant A33 polypeptide, wherein the variant A33 polypeptide provides for enhanced viral spreading and/or enhanced production of EEV, compared to a control vaccinia virus that does not comprise the nucleotide sequence encoding the variant A33 polypeptide (e.g., compared to a control vaccinia virus that comprises a nucleotide sequence encoding a wild-type A33 polypeptide, such as an A33 polypeptide having an amino acid sequence depicted in FIG. 1C). In some other embodiments, the present disclosure provides an OVV that comprises a nucleotide sequence encoding a variant A34 polypeptide, wherein the variant A34 polypeptide provides for enhanced viral spreading and/or enhanced production of EEV, compared to a control vaccinia virus that does not comprise the nucleotide sequence encoding the variant A34 polypeptide (e.g., compared to a control vaccinia virus that comprises a nucleotide sequence encoding a wild-type A34 polypeptide, such as an A34 polypeptide having an amino acid sequence depicted in FIG. 2C). In some further embodiments, the present disclosure provides an OVV that comprises a nucleotide sequence encoding a variant B5 polypeptide comprising one or more amino acid substitutions that provide for enhanced virus spreading and/or EEV production, compared to a control vaccinia virus that does not include a nucleotide sequence encoding a variant B5 polypeptide (e.g., compared to a control vaccinia virus that comprises a nucleotide sequence encoding a wild-type B5 polypeptide, such as a B5 polypeptide having an amino acid sequence depicted in FIG. 4D). In some still further embodiments, the present disclosure provides an OVV that comprises nucleotide sequence encoding a variant A33 polypeptide, a nucleotide sequence encoding a variant A34 polypeptide, and a nucleotide sequence encoding a variant B5 polypeptide in any combinations. Examples of suitable controls include IGV-007 and IGV-006, as described in the Examples. Examples of variant A33 polypeptides, variant A34 polypeptides, and variant B5 polypeptides that provide for enhanced virus spreading and/or EEV production are described in detail below.

In some cases, an OVV of the present disclosure comprises a) a nucleotide sequence encoding a variant A33 polypeptide, wherein the variant A33 polypeptide provides for enhanced viral spreading and/or enhanced production of EEV, compared to a control vaccinia virus that does not comprise the nucleotide sequence encoding the variant A33 polypeptide; b) a nucleotide sequence encoding an A34 polypeptide that does not include any amino acid substitutions that provide for enhanced viral spreading and/or enhanced production of EEV; and c) a nucleotide sequence encoding a B5 polypeptide that does not include any amino acid substitutions that provide for enhanced viral spreading and/or enhanced production of EEV. In some cases, an OVV of the present disclosure comprises: a) a nucleotide sequence encoding a variant A33 polypeptide, wherein the variant A33 polypeptide provides for enhanced viral spreading and/or enhanced production of EEV, compared to a control vaccinia virus that does not comprise the nucleotide sequence encoding the variant A33 polypeptide; b) a nucleotide sequence encoding a wild-type A34 polypeptide (e.g., an A34 polypeptide having a naturally-occurring amino acid sequence); and c) a nucleotide sequence encoding a wild-type B5 polypeptide (e.g., a B5 polypeptide having a naturally-occurring amino acid sequence).

In some cases, an OVV of the present disclosure comprises: a) a nucleotide sequence encoding a variant A34 polypeptide, wherein the variant A34 polypeptide provides for enhanced viral spreading and/or enhanced production of EEV, compared to a control vaccinia virus that does not comprise the nucleotide sequence encoding the variant A34 polypeptide; b) a nucleotide sequence encoding an A33 polypeptide that does not include any amino acid substitutions that provide for enhanced viral spreading and/or enhanced production of EEV (e.g., a wild-type A33 polypeptide); and c) a nucleotide sequence encoding a B5 polypeptide that does not include any amino acid substitutions that provide for enhanced viral spreading and/or enhanced production of EEV (e.g., a wild-type B5 polypeptide). In some cases, an OVV of the present disclosure comprises: a) a nucleotide sequence encoding a variant A34 polypeptide, wherein the variant A34 polypeptide provides for enhanced viral spreading and/or enhanced production of EEV, compared to a control vaccinia virus that does not comprise the nucleotide sequence encoding the variant A34 polypeptide; b) a nucleotide sequence encoding a wild-type A33 polypeptide (e.g., an A33 polypeptide having a naturally-occurring amino acid sequence); and c) a nucleotide sequence encoding a wild-type B5 polypeptide (e.g., a B5 polypeptide having a naturally-occurring amino acid sequence).

In some cases, an OVV of the present disclosure comprises: a) a nucleotide sequence encoding a variant B5 polypeptide, wherein the variant B5 polypeptide provides for enhanced viral spreading and/or enhanced production of EEV, compared to a control vaccinia virus that does not comprise the nucleotide sequence encoding the variant B5 polypeptide; b) a nucleotide sequence encoding an A33 polypeptide that does not include any amino acid substitutions that provide for enhanced viral spreading and/or enhanced production of EEV; and c) a nucleotide sequence encoding an A34 polypeptide that does not include any amino acid substitutions that provide for enhanced viral spreading and/or enhanced production of EEV. In some cases, an OVV of the present disclosure comprises: a) a nucleotide sequence encoding a variant B5 polypeptide, wherein the variant B5 polypeptide provides for enhanced viral spreading and/or enhanced production of EEV, compared to a control vaccinia virus that does not comprise the nucleotide sequence encoding the variant B5 polypeptide; b) a nucleotide sequence encoding a wild-type A33 polypeptide (e.g., an A33 polypeptide having a naturally-occurring amino acid sequence); and c) a nucleotide sequence encoding a wild-type A34 polypeptide (e.g., an A34 polypeptide having a naturally-occurring amino acid sequence).

In some cases, an OVV of the present disclosure comprises: a) a nucleotide sequence encoding a variant A33 polypeptide, wherein the variant A33 polypeptide provides for enhanced viral spreading and/or enhanced production of EEV, compared to a control vaccinia virus that does not comprise the nucleotide sequence encoding the variant A33 polypeptide (e.g., compared to a control vaccinia virus that comprises a nucleotide sequence encoding a wild-type A33 polypeptide, such as an A33 polypeptide having an amino acid sequence depicted in FIG. 1C); b) a nucleotide sequence encoding a variant A34 polypeptide, wherein the variant A34 polypeptide provides for enhanced viral spreading and/or enhanced production of EEV, compared to a control vaccinia virus that does not comprise the nucleotide sequence encoding the variant A34 polypeptide (e.g., compared to a control vaccinia virus that comprises a nucleotide sequence encoding a wild-type A34 polypeptide, such as an A34 polypeptide having an amino acid sequence depicted in FIG. 2C); and c) a nucleotide sequence encoding a wild-type B5 polypeptide (e.g., a B5 polypeptide having a naturally-occurring amino acid sequence).

In some cases, an OVV of the present disclosure comprises: a) a nucleotide sequence encoding a variant A33 polypeptide, wherein the variant A33 polypeptide provides for enhanced viral spreading and/or enhanced production of EEV, compared to a control vaccinia virus that does not comprise the nucleotide sequence encoding the variant A33 polypeptide (e.g., compared to a control vaccinia virus that comprises a nucleotide sequence encoding a wild-type A33 polypeptide, such as an A33 polypeptide having an amino acid sequence depicted in FIG. 1C); b) a nucleotide sequence encoding a variant B5 polypeptide, wherein the variant B5 polypeptide provides for enhanced viral spreading and/or enhanced production of EEV, compared to a control vaccinia virus that does not comprise the nucleotide sequence encoding the variant B5 polypeptide (e.g., compared to a control vaccinia virus that comprises a nucleotide sequence encoding a wild-type B5 polypeptide, such as an B5 polypeptide having an amino acid sequence depicted in FIG. 4D); and c) a nucleotide sequence encoding a wild-type A34 polypeptide (e.g., an A34 polypeptide having a naturally-occurring amino acid sequence).

In some cases, an OVV of the present disclosure comprises: a) a nucleotide sequence encoding a variant A34 polypeptide, wherein the variant A34 polypeptide provides for enhanced viral spreading and/or enhanced production of EEV, compared to a control vaccinia virus that does not comprise the nucleotide sequence encoding the variant A34 polypeptide (e.g., compared to a control vaccinia virus that comprises a nucleotide sequence encoding a wild-type A34 polypeptide, such as an A34 polypeptide having an amino acid sequence depicted in FIG. 2C); b) a nucleotide sequence encoding a variant B5 polypeptide, wherein the variant B5 polypeptide provides for enhanced viral spreading and/or enhanced production of EEV, compared to a control vaccinia virus that does not comprise the nucleotide sequence encoding the variant B5 polypeptide (e.g., compared to a control vaccinia virus that comprises a nucleotide sequence encoding a wild-type B5 polypeptide, such as a B5 polypeptide having an amino acid sequence depicted in FIG. 4D); and c) a nucleotide sequence encoding a wild-type A33 polypeptide (e.g., an A33 polypeptide having a naturally-occurring amino acid sequence).

In some cases, an OVV of the present disclosure comprises: a) a nucleotide sequence encoding a variant A33 polypeptide, wherein the variant A33 polypeptide provides for enhanced viral spreading and/or enhanced production of EEV, compared to a control vaccinia virus that does not comprise the nucleotide sequence encoding the variant A33 polypeptide (e.g., compared to a control vaccinia virus that comprises a nucleotide sequence encoding a wild-type A33 polypeptide, such as an A33 polypeptide having an amino acid sequence depicted in FIG. 1C); b) a nucleotide sequence encoding a variant A34 polypeptide, wherein the variant A34 polypeptide provides for enhanced viral spreading and/or enhanced production of EEV, compared to a control vaccinia virus that does not comprise the nucleotide sequence encoding the variant A34 polypeptide (e.g., compared to a control vaccinia virus that comprises a nucleotide sequence encoding a wild-type A34 polypeptide, such as an A34 polypeptide having an amino acid sequence depicted in FIG. 2C); and c) a nucleotide sequence encoding a variant B5 polypeptide, wherein the variant B5 polypeptide provides for enhanced viral spreading and/or enhanced production of EEV, compared to a control vaccinia virus that does not comprise the nucleotide sequence encoding the variant B5 polypeptide (e.g., compared to a control vaccinia virus that comprises a nucleotide sequence encoding a wild-type B5 polypeptide, such as a B5 polypeptide having an amino acid sequence depicted in FIG. 4D).

In some cases, an OVV of the present disclosure exhibits increased viral spreading and/or increased EEV production, compared to a control vaccinia virus that does not comprise a nucleotide sequence encoding a variant A33 polypeptide and/or a variant A34 polypeptide and/or a variant B5 polypeptide, as described herein. For example, in some cases, an OVV of the present disclosure comprising a nucleotide sequence encoding a variant A33 polypeptide and/or a nucleotide sequence encoding a variant A34 polypeptide and/or a nucleotide sequence encoding a variant B5 polypeptide exhibits at least 10%, at least 15%, at least 20%, at least 25%, at least 50%, at least 75%, at least 100% (or 2-fold), at least 2.5-fold, at least 5-fold, at least 10-fold, at least 15-fold, at least 20-fold, at least 25-fold, or more than 25-fold, greater spreading, compared to a control vaccinia virus that does not comprise the nucleotide sequence(s) encoding the variant A33 polypeptide and/or the variant A34 polypeptide and/or the variant B5 polypeptide. Whether a given variant A33 polypeptide, a given variant A34 polypeptide, a given B5 polypeptide, or a combination of two or three of a given A33 polypeptide, a given variant A34 polypeptide, and a given variant B5 polypeptide, provides for increased viral spreading, compared to a control, can be determined using any known method, including a method as described in the Examples herein, such as the two-stage infectivity assay with U-2 OS cells described in Example 2. The two-stage infectivity assay comprises the steps of (1) infecting the U-2 cells with different 3-fold dilutions of multiplicities of infection (MOI) of vaccinia virus, (2) collecting the supernatant 22 hours post-infection, (3) infecting a new plate of U-2 OS cells with the supernatant, and (4) measuring luciferase expressed from the virus 15 hours post-infection, wherein an increased levels of luciferase is indicative of increased virus spreading.

In some cases, an OVV of the present disclosure comprising a nucleotide sequence encoding a variant A33 polypeptide, a nucleotide sequence encoding a variant A34 polypeptide, a nucleotide sequence encoding a variant B5 polypeptide, or nucleotide sequences encoding any combination of variant A33, A34, and B5 polypeptide exhibits at least 10%, at least 15%, at least 20%, at least 25%, at least 50%, at least 75%, at least 100% (or 2-fold), at least 2.5-fold, at least 5-fold, at least 10-fold, at least 15-fold, at least 20-fold, at least 25-fold, or more than 25-fold, greater EEV production, compared to a control vaccinia virus that comprise the nucleotide sequence(s) encoding the corresponding wild-type A33, A34, and B5 polypeptide. Whether a given variant A33 polypeptide, a given variant A34 polypeptide, a given variant B5 polypeptide, or a combination of two or three of a given A33 polypeptide, a given variant A34 polypeptide, and a given B5 polypeptide, provides for increased EEV production, compared to a control, can be determined using any known method, including a method as described in the Examples herein, such as the plaque assay described in Example 3. The plaque assay comprises the steps of (1) infecting one or more cell lines the viruses, (2) washing the cells after 1 hour of virus adsorption, (3) collecting the supernatant (potentially EEVs). at 24 hours post-infection, and (4) determining the number of infectious viruses produced in the supernatant via plaque assay.

In some cases, an OVV of the present disclosure exhibits increased viral spreading and/or increased EEV production, compared to a control vaccinia virus that does not comprise a nucleotide sequence encoding a variant A33 polypeptide and/or a variant A34 polypeptide as described herein. For example, in some cases, an OVV of the present disclosure comprising a nucleotide sequence encoding a variant A33 polypeptide and/or a nucleotide sequence encoding a variant A34 polypeptide exhibits at least 10%, at least 15%, at least 20%, at least 25%, at least 50%, at least 75%, at least 100% (or 2-fold), at least 2.5-fold, at least 5-fold, at least 10-fold, at least 15-fold, at least 20-fold, at least 25-fold, or more than 25-fold, greater spreading, compared to a control vaccinia virus that does not comprise the nucleotide sequence(s) encoding the variant A33 polypeptide and/or the variant A34 polypeptide (e.g., compared to a control vaccinia virus that comprises a nucleotide sequence encoding a wild-type A33 polypeptide, such as an A33 polypeptide having an amino acid sequence depicted in FIG. 1C); and/or that comprises a nucleotide sequence encoding a wild-type A34 polypeptide, such as an A34 polypeptide having an amino acid sequence depicted in FIG. 2C). Whether a given variant A33 polypeptide, a given variant A34 polypeptide, or a combination of a given A33 polypeptide and a given variant A34 polypeptide, provides for increased viral spreading, compared to a control, can be determined using any known method, including a method as described in the Examples herein.

In some cases, an OVV of the present disclosure comprising a nucleotide sequence encoding a variant A33 polypeptide and/or a nucleotide sequence encoding a variant A34 polypeptide exhibits at least 10%, at least 15%, at least 20%, at least 25%, at least 50%, at least 75%, at least 100% (or 2-fold), at least 2.5-fold, at least 5-fold, at least 10-fold, at least 15-fold, at least 20-fold, at least 25-fold, or more than 25-fold, greater EEV production, compared to a control vaccinia virus that does not comprise the nucleotide sequence(s) encoding the variant A33 polypeptide and/or the variant A34 polypeptide (e.g., compared to a control vaccinia virus that comprises a nucleotide sequence encoding a wild-type A33 polypeptide, such as an A33 polypeptide having an amino acid sequence depicted in FIG. 1C); and/or that comprises a nucleotide sequence encoding a wild-type A34 polypeptide, such as an A34 polypeptide having an amino acid sequence depicted in FIG. 2C). Whether a given variant A33 polypeptide, a given variant A34 polypeptide, or a combination of a given A33 polypeptide and a given variant A34 polypeptide, provides for increased EEV production, compared to a control, can be determined using any known method, including a method as described in the Examples herein.

In some cases, an OVV of the present disclosure exhibits increased viral spreading and/or increased EEV production, compared to a control vaccinia virus that does not comprise a nucleotide sequence encoding a variant B5 polypeptide as described herein (e.g., compared to a control vaccinia virus that comprises a nucleotide sequence encoding a wild-type B5 polypeptide, such as a B5 polypeptide having an amino acid sequence depicted in FIG. 4D). For example, in some cases, an OVV of the present disclosure comprising a nucleotide sequence encoding a variant B5 polypeptide exhibits at least 10%, at least 15%, at least 20%, at least 25%, at least 50%, at least 75%, at least 100% (or 2-fold), at least 2.5-fold, at least 5-fold, at least 10-fold, at least 15-fold, at least 20-fold, at least 25-fold, or more than 25-fold, greater spreading, compared to a control vaccinia virus that does not include a nucleotide sequence encoding a variant B5 polypeptide (e.g., compared to a control vaccinia virus that comprises a nucleotide sequence encoding a wild-type B5 polypeptide, such as a B5 polypeptide having an amino acid sequence depicted in FIG. 4D). Whether a given variant B5 polypeptide provides for increased viral spreading, compared to a control, can be determined using any known method, including a method as described in the Examples herein.

In some cases, an OVV of the present disclosure comprising a nucleotide sequence encoding a variant B5 polypeptide exhibits at least 10%, at least 15%, at least 20%, at least 25%, at least 50%, at least 75%, at least 100% (or 2-fold), at least 2.5-fold, at least 5-fold, at least 10-fold, at least 15-fold, at least 20-fold, at least 25-fold, or more than 25-fold, greater EEV production, compared to a control vaccinia virus that does not include a nucleotide sequence encoding a variant B5 polypeptide (e.g., compared to a control vaccinia virus that comprises a nucleotide sequence encoding a wild-type B5 polypeptide, such as a B5 polypeptide having an amino acid sequence depicted in FIG. 4D). Whether a given variant B5 polypeptide provides for increased EEV production, compared to a control, can be determined using any known method, including a method as described in the Examples herein.

In some cases, at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, or more than 75%, of the infectious virions produced using an OVV of the present disclosure are EEV. In some cases, at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, or more than 75%, of the total physical viral particles produced using an OVV of the present disclosure are EEV.

Variant A33 Polypeptides

Examples of wild-type amino acid sequences of A33 polypeptides of common strains of vaccinia virus are depicted in FIG. 1C and set forth in SEQ ID NOs:8-14. A variant A33 polypeptide can comprise 1, 2, 3, or more amino acid mutations, such as substitutions at one or more of M63, A88, and E129, where the amino acid numbering is based on the numbering shown in FIG. 1C (e.g., where the amino acid numbering is based on the amino acid numbering of Copenhagen A33 amino acid sequence; SEQ ID NO:8).

In some cases, a variant A33 polypeptide comprises an amino acid sequence having at least 95%, at least 96%, at least 97%, at least 98%, or at least 99%, amino acid sequence identity to an A33 amino acid sequence depicted in FIG. 1C; and comprises an amino acid at position 63 other than Met. For example, in some cases, a variant A33 polypeptide comprises Ala, Gly, lie, Leu, Pro, Val, Phe, Trp, Tyr, Asp, Glu, Arg, His, Lys, Ser, Thr, Cys, Asn, or Gln at position 63, based on the amino acid numbering depicted in FIG. 1C. In some cases, a variant A33 polypeptide comprises an M63R substitution, an M63H, or an M63K substitution. In some cases, a variant A33 polypeptide comprises an M63R substitution.

In some cases, a variant A33 polypeptide comprises an amino acid sequence having at least 95%, at least 96%, at least 97%, at least 98%, or at least 99%, amino acid sequence identity to an A33 amino acid sequence depicted in FIG. 1C; and comprises an amino acid at position 88 other than Ala. For example, in some cases, a variant A33 polypeptide comprises Gly, lie, Leu, Pro, Val, Phe, Trp, Tyr, Asp, Glu, Arg, His, Lys, Ser, Thr, Cys, Met, Asn, or Gln at position 88, based on the amino acid numbering depicted in FIG. 1C. In some cases, a variant A33 polypeptide comprises an A88D substitution or an A88E substitution. In some cases, a variant A33 polypeptide comprises an A88D substitution.

In some cases, a variant A33 polypeptide comprises an amino acid sequence having at least 95%, at least 96%, at least 97%, at least 98%, or at least 99%, amino acid sequence identity to an A33 amino acid sequence depicted in FIG. 1C; and comprises an amino acid at position 129 other than Glu. For example, in some cases, a variant A33 polypeptide comprises Ala, Gly, lie, Leu, Pro, Val, Phe, Trp, Tyr, Asp, Arg, His, Lys, Ser, Thr, Cys, Met, Asn, or Gln at position 129, based on the amino acid numbering depicted in FIG. 1C. In some cases, a variant A33 polypeptide comprises an E129M substitution.

In some cases, a variant A33 polypeptide comprises an amino acid sequence having at least 95%, at least 96%, at least 97%, at least 98%, or at least 99%, amino acid sequence identity to an A33 amino acid sequence depicted in FIG. 1C; comprises an amino acid at position 63 other than Met, based on the amino acid numbering depicted in FIG. 1C; and comprises an amino acid at position 88 other than Ala, based on the amino acid numbering depicted in FIG. 1C. In some cases, a variant A33 polypeptide comprises an amino acid sequence having at least 95%, at least 96%, at least 97%, at least 98%, or at least 99%, amino acid sequence identity to an A33 amino acid sequence depicted in FIG. 1C; and comprises an M63R substitution and an A88D substitution.

In some cases, a variant A33 polypeptide comprises an amino acid sequence having at least 95%, at least 96%, at least 97%, at least 98%, or at least 99%, amino acid sequence identity to an A33 amino acid sequence depicted in FIG. 1C; comprises an amino acid at position 63 other than Met, based on the amino acid numbering depicted in FIG. 1C; and comprises an amino acid at position 129 other than Glu, based on the amino acid numbering depicted in FIG. 1C. In some cases, a variant A33 polypeptide comprises an amino acid sequence having at least 95%, at least 96%, at least 97%, at least 98%, or at least 99%, amino acid sequence identity to an A33 amino acid sequence depicted in FIG. 1C; and comprises an M63R substitution and an E129M substitution.

In some cases, a variant A33 polypeptide comprises an amino acid sequence having at least 95%, at least 96%, at least 97%, at least 98%, or at least 99%, amino acid sequence identity to an A33 amino acid sequence depicted in FIG. 1C; comprises an amino acid at position 88 other than Ala, based on the amino acid numbering depicted in FIG. 1C; and comprises an amino acid at position 129 other than Glu, based on the amino acid numbering depicted in FIG. 1C. In some cases, a variant A33 polypeptide comprises an amino acid sequence having at least 95%, at least 96%, at least 97%, at least 98%, or at least 99%, amino acid sequence identity to an A33 amino acid sequence depicted in FIG. 1C; and comprises an A88D substitution and an E129M substitution.

In some cases, a variant A33 polypeptide comprises an amino acid sequence having at least 95%, at least 96%, at least 97%, at least 98%, or at least 99%, amino acid sequence identity to an A33 amino acid sequence depicted in FIG. 1C; comprises an amino acid at position 63 other than Met, based on the amino acid numbering depicted in FIG. 1C; comprises an amino acid at position 88 other than Ala, based on the amino acid numbering depicted in FIG. 1C; and comprises an amino acid at position 88 other than Ala, based on the amino acid numbering depicted in FIG. 1C; and comprises an amino acid at position 129 other than Glu, based on the amino acid numbering depicted in FIG. 1C. In some cases, a variant A33 polypeptide comprises an amino acid sequence having at least 95%, at least 96%, at least 97%, at least 98%, or at least 99%, amino acid sequence identity to an A33 amino acid sequence depicted in FIG. 1C; and comprises an M63R substitution, an A88D substitution, and an E129M substitution.

Variant A34 Polypeptides

Examples of wild-type amino acid sequences of A34 polypeptides of common strains of vaccinia virus are depicted in FIG. 2C and set forth in SEQ ID NOs:22-28. A variant A34 polypeptide can comprise 1, 2, 3, 4, 5, 6, or more amino acid mutations, such as amino acid substitutions at 1, 2, 3, 4, or 5 of M66, F94, R84, R91, and T127, where the amino acid numbering is based on the numbering shown in FIG. 2C (e.g., where the amino acid numbering is based on the amino acid numbering of Copenhagen A34 amino acid sequence; SEQ ID NO:22).

In some cases, a variant A34 polypeptide comprises an amino acid sequence having at least 95%, at least 96%, at least 97%, at least 98%, or at least 99%, amino acid sequence identity to an A34 amino acid sequence depicted in FIG. 2C; and comprises an amino acid at position 66 other than Met. For example, in some cases, a variant A34 polypeptide comprises Ala, Gly, lie, Leu, Pro, Val, Phe, Trp, Tyr, Asp, Glu, Arg, His, Lys, Ser, Thr, Cys, Asn, or Gln at position 66, based on the amino acid numbering depicted in FIG. 2C. In some cases, a variant A34 polypeptide comprises an M66T substitution or an M66S substitution. In some cases, a variant A34 polypeptide comprises an M66T substitution. In some cases, the variant A34 polypeptide comprises a Lys at position 151. In some cases, the variant A34 polypeptide comprises a Glu at position 151.

In some cases, a variant A34 polypeptide comprises an amino acid sequence having at least 95%, at least 96%, at least 97%, at least 98%, or at least 99%, amino acid sequence identity to an A34 amino acid sequence depicted in FIG. 2C; and comprises an amino acid at position 94 other than Phe. For example, in some cases, a variant A34 polypeptide comprises Ala, Gly, lie, Leu, Pro, Val, Trp, Tyr, Asp, Glu, Arg, His, Lys, Ser, Thr, Cys, Met, Asn, or Gln at position 94, based on the amino acid numbering depicted in FIG. 2C. In some cases, a variant A34 polypeptide comprises an F94H substitution, an F94R substitution, or an F94K substitution. In some cases, a variant A34 polypeptide comprises an F94H substitution. In some cases, the variant A34 polypeptide comprises a Lys at position 151. In some cases, the variant A34 polypeptide comprises a Glu at position 151.

In some cases, a variant A34 polypeptide comprises an amino acid sequence having at least 95%, at least 96%, at least 97%, at least 98%, or at least 99%, amino acid sequence identity to an A34 amino acid sequence depicted in FIG. 2C; and comprises an amino acid at position 84 other than Arg. For example, in some cases, a variant A34 polypeptide comprises Ala, Gly, lie, Leu, Pro, Val, Phe, Trp, Tyr, Asp, Glu, His, Lys, Ser, Thr, Cys, Met, Asn, or Gln at position 84, based on the amino acid numbering depicted in FIG. 2C. In some cases, a variant A34 polypeptide comprises an R84G substitution, an R84A substitution, an R841 substitution, an R84L substitution, or an R84V substitution. In some cases, a variant A34 polypeptide comprises an R84G substitution. In some cases, the variant A34 polypeptide comprises a Lys at position 151. In some cases, the variant A34 polypeptide comprises a Glu at position 151.

In some cases, a variant A34 polypeptide comprises an amino acid sequence having at least 95%, at least 96%, at least 97%, at least 98%, or at least 99%, amino acid sequence identity to an A34 amino acid sequence depicted in FIG. 2C; and comprises an amino acid at position 91 other than Arg. For example, in some cases, a variant A34 polypeptide comprises Ala, Gly, lie, Leu, Pro, Val, Phe, Trp, Tyr, Asp, Glu, His, Lys, Ser, Thr, Cys, Met, Asn, or Gln at position 91, based on the amino acid numbering depicted in FIG. 2C. In some cases, a variant A34 polypeptide comprises an R91S substitution, an R91A substitution, or an R91T substitution. In some cases, a variant A34 polypeptide comprises an R91S substitution. In some cases, a variant A34 polypeptide comprises an R91A substitution. In some cases, the variant A34 polypeptide comprises a Lys at position 151. In some cases, the variant A34 polypeptide comprises a Glu at position 151.

In some cases, a variant A34 polypeptide comprises an amino acid sequence having at least 95%, at least 96%, at least 97%, at least 98%, or at least 99%, amino acid sequence identity to an A34 amino acid sequence depicted in FIG. 2C; and comprises an amino acid at position 127 other than Thr. For example, in some cases, a variant A34 polypeptide comprises Ala, Gly, lie, Leu, Pro, Val, Phe, Trp, Tyr, Asp, Glu, Arg, His, Lys, Ser, Cys, Met, Asn, or Gln at position 127, based on the amino acid numbering depicted in FIG. 2C. In some cases, a variant A34 polypeptide comprises a T127E substitution or a T127D substitution. In some cases, a variant A34 polypeptide comprises a T127E substitution. In some cases, the variant A34 polypeptide comprises a Lys at position 151. In some cases, the variant A34 polypeptide comprises a Glu at position 151.

In some cases, a variant A34 polypeptide comprises an amino acid sequence having at least 95%, at least 96%, at least 97%, at least 98%, or at least 99%, amino acid sequence identity to an A34 amino acid sequence depicted in FIG. 2C; and comprises amino acid substitutions at 2 positions selected from M66, F94, R84, R91, and T127, where the amino acid numbering is based on the numbering shown in FIG. 2C. Thus, e.g., in some cases, a variant A34 comprises substitutions at M66 and F94; at M66 and R84; at M66 and R91; at M66 and T127; at F94 and R84; and F94 and F91; at F94 and T127; at R84 and R91; at R84 and T127; or at R91 and T127. In some cases, the substitution at M66 is an M66T substitution. In some cases, the substitution at F94 is an F94H substitution. In some cases, the substitution at R84 is an R84G substitution. In some cases, the substitution at R91 is an R91S substitution. In some cases, the substitution at R91 is an R91A substitution. In some cases, the substitution at T127 is a T127E substitution. In some cases, the variant A34 polypeptide comprises a Lys at position 151. In some cases, the variant A34 polypeptide comprises a Glu at position 151.

In some cases, a variant A34 polypeptide comprises an amino acid sequence having at least 95%, at least 96%, at least 97%, at least 98%, or at least 99%, amino acid sequence identity to an A34 amino acid sequence depicted in FIG. 2C; and comprises amino acid substitutions at 3 positions selected from M66, F94, R84, R91, and T127, where the amino acid numbering is based on the numbering shown in FIG. 2C. Thus, e.g., in some cases, a variant A34 comprises substitutions at M66, F94, and R84; at M66, F94, and R91; at M66, F94, and T127; at F94, R84, and R91; at F94, R84, and T127; at F94, R91, and T127; at R84, R91, and T127; at M66, R84, and R91; or at M66, R91, and T127. In some cases, the substitution at M66 is an M66T substitution. In some cases, the substitution at F94 is an F94H substitution. In some cases, the substitution at R84 is an R84G substitution. In some cases, the substitution at R91 is an R91S substitution. In some cases, the substitution at R91 is an R91A substitution. In some cases, the substitution at T127 is a T127E substitution. In some cases, the variant A34 polypeptide comprises a Lys at position 151. In some cases, the variant A34 polypeptide comprises a Glu at position 151.

In some cases, a variant A34 polypeptide comprises an amino acid sequence having at least 95%, at least 96%, at least 97%, at least 98%, or at least 99%, amino acid sequence identity to an A34 amino acid sequence depicted in FIG. 2C; and comprises amino acid substitutions at 4 positions selected from M66, F94, R84, R91, and T127, where the amino acid numbering is based on the numbering shown in FIG. 2C. Thus, e.g., in some cases, a variant A34 comprises substitutions at M66, F94, R84, and R91; at M66, F94, R84, and T127; at F94, R84, R91, and T127; at M66, R84, R91, and T127; or at M66, F94, R91, and T127. In some cases, the substitution at M66 is an M66T substitution. In some cases, the substitution at F94 is an F94H substitution. In some cases, the substitution at R84 is an R84G substitution. In some cases, the substitution at R91 is an R91S substitution. In some cases, the substitution at R91 is an R91A substitution. In some cases, the substitution at T127 is a T127E substitution. In some cases, the variant A34 polypeptide comprises a Lys at position 151. In some cases, the variant A34 polypeptide comprises a Glu at position 151.

In any of the above-described embodiments, a variant A34 polypeptide can have, at position 151 based on the numbering shown in FIG. 2C, a Lys. In any of the above-described embodiments, a variant A34 polypeptide can have, at position 151 based on the numbering shown in FIG. 2C, an amino acid other than Lys. In any of the above-described embodiments, a variant A34 polypeptide can have, at position 151 based on the numbering shown in FIG. 2C, a Glu. Thus, e.g., in some cases, a variant A34 polypeptide comprises an amino acid sequence having at least 95%, at least 96%, at least 97%, at least 98%, or at least 99%, amino acid sequence identity to an A34 amino acid sequence depicted in FIG. 2C; and comprises an amino acid substitution at F94 and an amino acid substitution at K151. For example, in some cases, a variant A34 polypeptide comprises an F94H substitution and a K151E substitution.

Combinations of Variant A33 Polypeptide and Variant A34 Polypeptide

As noted above, in some cases, an OVV of the present disclosure comprises: a) a nucleotide sequence encoding a variant A33 polypeptide, where the variant A33 polypeptide provides for enhanced viral spreading and/or enhanced production of EEV, compared to a control vaccinia virus that does not comprise the nucleotide sequence encoding the variant A33 polypeptide (e.g., compared to a control vaccinia virus that comprises a nucleotide sequence encoding a wild-type A33 polypeptide, such as an A33 polypeptide having an amino acid sequence depicted in FIG. 1C); and b) a nucleotide sequence encoding a variant A34 polypeptide, where the variant A34 polypeptide provides for enhanced viral spreading and/or enhanced production of EEV, compared to a control vaccinia virus that does not comprise the nucleotide sequence encoding the variant A34 polypeptide (e.g., compared to a control vaccinia virus that comprises a nucleotide sequence encoding a wild-type A34 polypeptide, such as an A34 polypeptide having an amino acid sequence depicted in FIG. 2C).

An OVV of the present disclosure that comprises nucleotide sequences encoding both a variant A33 polypeptide and a variant A34 polypeptide can comprise nucleotide sequences encoding any of the above-described variant A33 polypeptides and any of the above-described A34 polypeptides in any combination. Non-limiting examples of combinations are set out in Table 2.

Thus, e.g., in some cases, an OVV of the present disclosure comprises: a) a nucleotide sequence encoding a variant A33 polypeptide, where the variant A33 polypeptide comprises 1, 2, or 3 amino acid substitutions at M63, A88, and E129, as described above; and b) a nucleotide sequence encoding a variant A34 polypeptide, where the variant A34 polypeptide comprises 1, 2, 3, 4, or 5 amino acid substitutions at M66, F94, R84, R91, and T127, as described above.

The following are exemplary combinations and are not meant to be limiting.

A33(A88x) and A34(F94x)

As an example, an OVV of the present disclosure comprises a) a nucleotide sequence encoding a variant A33 polypeptide, where the variant A33 polypeptide comprises an amino acid sequence having at least 95%, at least 96%, at least 97%, at least 98%, or at least 99%, amino acid sequence identity to an A33 amino acid sequence depicted in FIG. 1C; and comprises an amino acid at position 88 other than Ala, based on the amino acid numbering depicted in FIG. 1C (e.g., the variant A33 polypeptide comprises Gly, lie, Leu, Pro, Val, Phe, Trp, Tyr, Asp, Glu, Arg, His, Lys, Ser, Thr, Cys, Met, Asn, or Gln at position 88); and b) a nucleotide sequence encoding a variant A34 polypeptide, where the variant A34 polypeptide comprises an amino acid sequence having at least 95%, at least 96%, at least 97%, at least 98%, or at least 99%, amino acid sequence identity to an A34 amino acid sequence depicted in FIG. 2C; and comprises an amino acid at position 94 other than Phe (e.g., the variant A34 polypeptide comprises Ala, Gly, lie, Leu, Pro, Val, Trp, Tyr, Asp, Glu, Arg, His, Lys, Ser, Thr, Cys, Met, Asn, or Gln at position 94). As an example, an OVV of the present disclosure comprises a) a nucleotide sequence encoding a variant A33 polypeptide, where the variant A33 polypeptide comprises an A88D substitution; and b) a nucleotide sequence encoding a variant A34 polypeptide, where the variant A34 polypeptide comprises an F94H substitution. In some cases, the variant A34 polypeptide comprises a Lys at position 151. In some cases, the variant A34 polypeptide comprises a Glu at position 151. For example, in some cases, an OVV of the present disclosure comprises a) a nucleotide sequence encoding a variant A33 polypeptide, where the variant A33 polypeptide comprises an A88D substitution; and b) a nucleotide sequence encoding a variant A34 polypeptide, where the variant A34 polypeptide comprises: i) an F94H substitution; and ii) a K151E substitution.

A33(E129x) AND A34(F94x) As an example, an OVV of the present disclosure comprises a) a nucleotide sequence encoding a variant A33 polypeptide, where the variant A33 polypeptide comprises an amino acid at position 129 other than Glu (e.g., the variant A33 polypeptide comprises Ala, Gly, lie, Leu, Pro, Val, Phe, Trp, Tyr, Asp, Arg, His, Lys, Ser, Thr, Cys, Met, Asn, or Gln at position 129; and b) a nucleotide sequence encoding a variant A34 polypeptide, where the variant A34 polypeptide comprises an amino acid sequence having at least 95%, at least 96%, at least 97%, at least 98%, or at least 99%, amino acid sequence identity to an A34 amino acid sequence depicted in FIG. 2C; and comprises an amino acid at position 94 other than Phe (e.g., the variant A34 polypeptide comprises Ala, Gly, lie, Leu, Pro, Val, Trp, Tyr, Asp, Glu, Arg, His, Lys, Ser, Thr, Cys, Met, Asn, or Gln at position 94). As an example, an OVV of the present disclosure comprises a) a nucleotide sequence encoding a variant A33 polypeptide, where the variant A33 polypeptide comprises an E129M substitution; and b) a nucleotide sequence encoding a variant A34 polypeptide, where the variant A34 polypeptide comprises an F94H substitution. In some cases, the variant A34 polypeptide comprises a Lys at position 151. In some cases, the variant A34 polypeptide comprises a Glu at position 151. Thus, for example, in some cases, an OVV of the present disclosure comprises a) a nucleotide sequence encoding a variant A33 polypeptide, where the variant A33 polypeptide comprises an E129M substitution; and b) a nucleotide sequence encoding a variant A34 polypeptide, where the variant A34 polypeptide comprises: i) an F94H substitution; and ii) a K151E substitution.

A33(A88x; and E129x) and A34(F94x)

As an example, an OVV of the present disclosure comprises: a) a nucleotide sequence encoding a variant A33 polypeptide, where the variant A33 polypeptide comprises an amino acid sequence having at least 95%, at least 96%, at least 97%, at least 98%, or at least 99%, amino acid sequence identity to an A33 amino acid sequence depicted in FIG. 1C; and comprises: i) an amino acid at position 88 other than Ala, based on the amino acid numbering depicted in FIG. 1C (e.g., the variant A33 polypeptide comprises Gly, lie, Leu, Pro, Val, Phe, Trp, Tyr, Asp, Glu, Arg, His, Lys, Ser, Thr, Cys, Met, Asn, or Gln at position 88); and ii) an amino acid at position 129 other than Glu (e.g., the variant A33 polypeptide comprises Ala, Gly, lie, Leu, Pro, Val, Phe, Trp, Tyr, Asp, Arg, His, Lys, Ser, Thr, Cys, Met, Asn, or Gln at position 129; and b) a nucleotide sequence encoding a variant A34 polypeptide, where the variant A34 polypeptide comprises an amino acid sequence having at least 95%, at least 96%, at least 97%, at least 98%, or at least 99%, amino acid sequence identity to an A34 amino acid sequence depicted in FIG. 2C; and comprises an amino acid at position 94 other than Phe (e.g., the variant A34 polypeptide comprises Ala, Gly, lie, Leu, Pro, Val, Trp, Tyr, Asp, Glu, Arg, His, Lys, Ser, Thr, Cys, Met, Asn, or Gln at position 94). As an example, an OVV of the present disclosure comprises a) a nucleotide sequence encoding a variant A33 polypeptide, where the variant A33 polypeptide comprises: i) an A88D substitution; and ii) an E129M substitution; and b) a nucleotide sequence encoding a variant A34 polypeptide, where the variant A34 polypeptide comprises an F94H substitution. In some cases, the variant A34 polypeptide comprises a Lys at position 151. In some cases, the variant A34 polypeptide comprises a Glu at position 151. For example, in some cases, an OVV of the present disclosure comprises a) a nucleotide sequence encoding a variant A33 polypeptide, where the variant A33 polypeptide comprises: i) an A88D substitution; and ii) an E129M substitution; and b) a nucleotide sequence encoding a variant A34 polypeptide, where the variant A34 polypeptide comprises: i) an F94H substitution; and ii) a K151E substitution.

Variant A56 Polypeptides

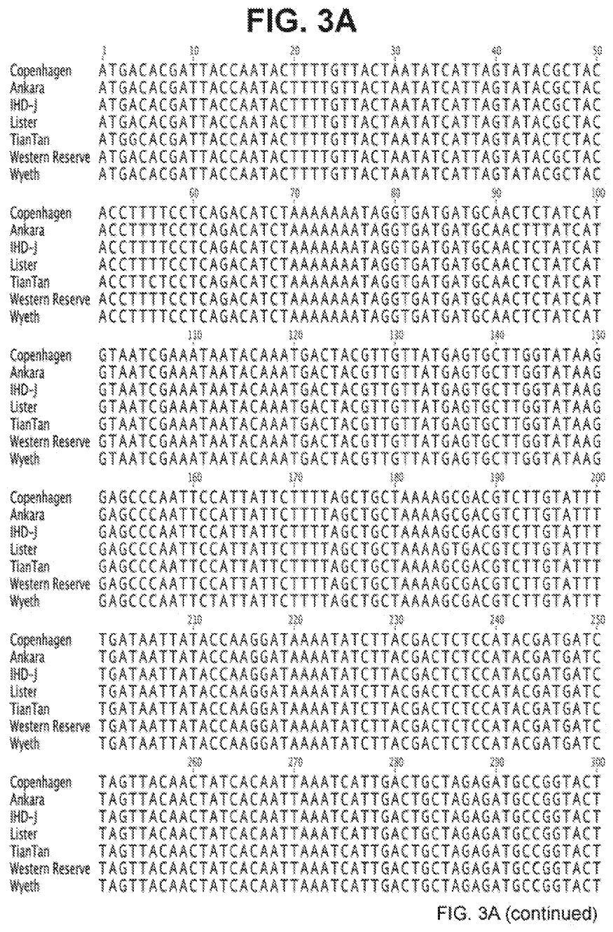

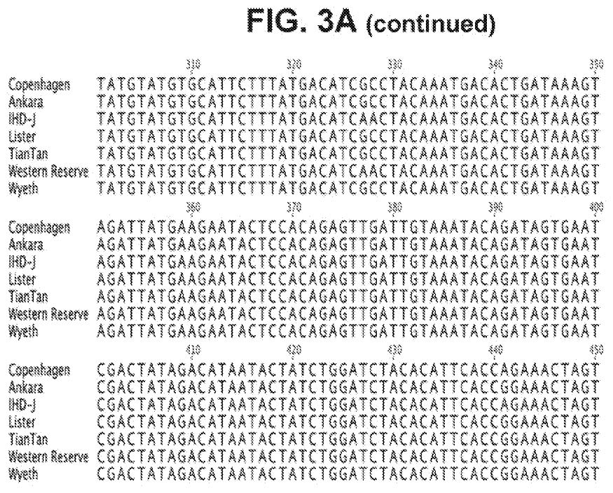

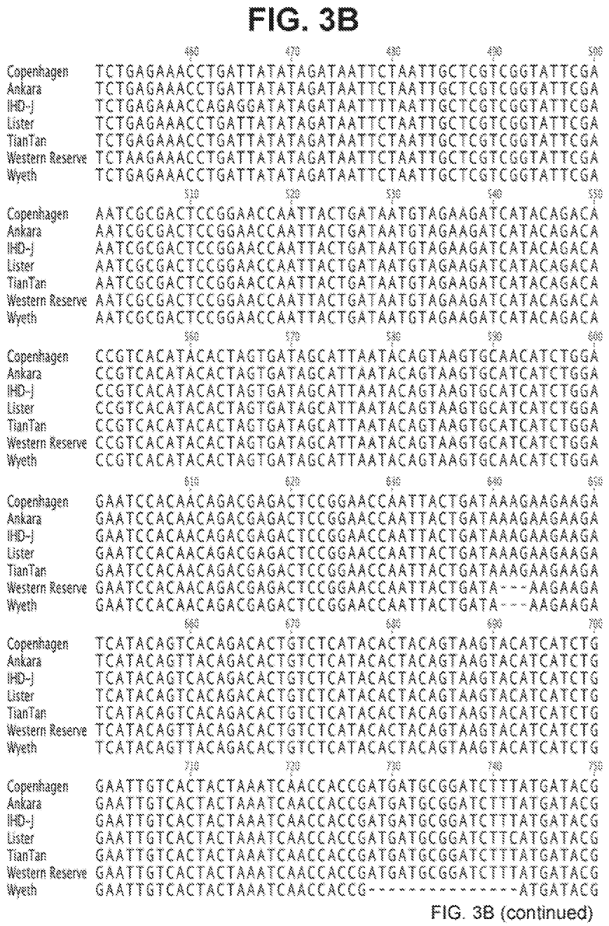

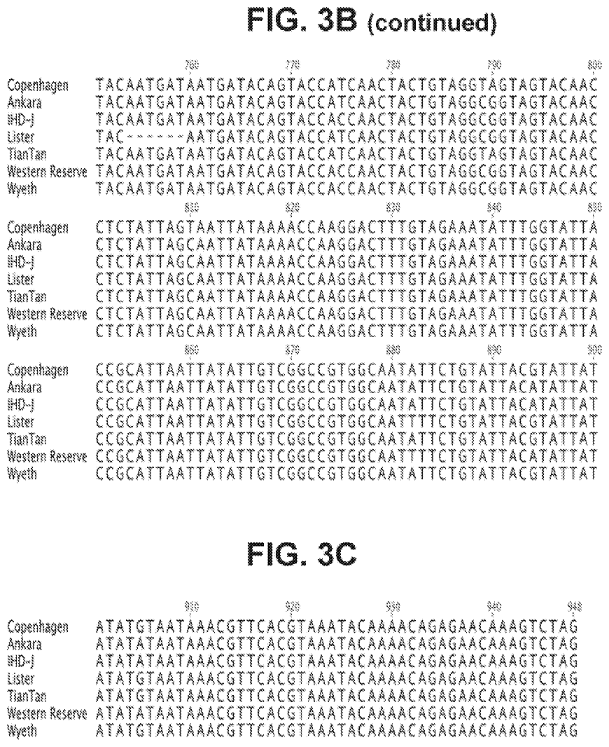

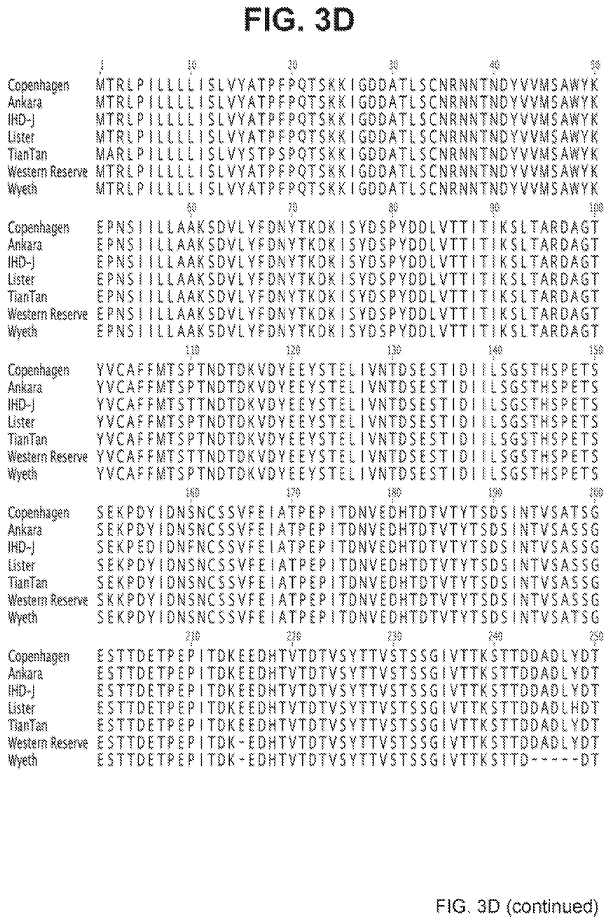

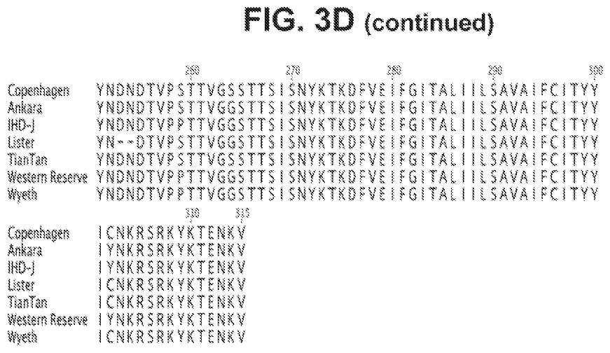

In any of the embodiments set out above, in some cases, an OVV of the present disclosure comprises a nucleotide sequence encoding a variant A56 polypeptide, where the A56 polypeptide comprises an amino acid sequence having at least 95%, at least 96%, at least 97%, at least 98%, or at least 99%, amino acid sequence identity to an A56 amino acid sequence depicted in FIG. 3D; where the encoded variant A56 polypeptide comprises an amino acid at position 269 other than lie, based on the amino acid numbering depicted in FIG. 3D (e.g., the variant A56 polypeptide comprises an Ala, Gly, Leu, Pro, Val, Phe, Trp, Tyr, Asp, Glu, Arg, His, Lys, Ser, Thr, Cys, Met, Asn, or Gln at position 269. For example, the variant A56 polypeptide can comprise Phe, Trp, or Tyr at amino acid 269. In some cases, the variant A56 polypeptide comprises a Phe at amino acid 269.

As an example, an OVV of the present disclosure comprises: a) a nucleotide sequence encoding a variant A34 polypeptide, where the variant A34 polypeptide comprises an amino acid sequence having at least 95%, at least 96%, at least 97%, at least 98%, or at least 99%, amino acid sequence identity to an A34 amino acid sequence depicted in FIG. 2C; and comprises an amino acid at position 91 other than Arg. For example, in some cases, a variant A34 polypeptide comprises Ala, Gly, lie, Leu, Pro, Val, Phe, Trp, Tyr, Asp, Glu, His, Lys, Ser, Thr, Cys, Met, Asn, or Gln at position 91, based on the amino acid numbering depicted in FIG. 2C; and b) a nucleotide sequence encoding a variant A56 polypeptide, where the A56 polypeptide comprises an amino acid sequence having at least 95%, at least 96%, at least 97%, at least 98%, or at least 99%, amino acid sequence identity to an A56 amino acid sequence depicted in FIG. 3D; where the encoded variant A56 polypeptide comprises an amino acid at position 269 other than lie, based on the amino acid numbering depicted in FIG. 3D (e.g., the variant A56 polypeptide comprises an Ala, Gly, Leu, Pro, Val, Phe, Trp, Tyr, Asp, Glu, Arg, His, Lys, Ser, Thr, Cys, Met, Asn, or Gln at position 269. For example, an OVV of the present disclosure can comprise: a) a nucleotide sequence encoding a variant A34 polypeptide, where the variant A34 polypeptide comprises an R91S substitution; and b) a nucleotide sequence encoding a variant A56 polypeptide, where the variant A56 polypeptide comprises an I269F substitution. In some cases, the variant A34 polypeptide comprises a Lys at position 151. In some cases, the variant A34 polypeptide comprises a Glu at position 151. For example, an OVV of the present disclosure can comprise: a) a nucleotide sequence encoding a variant A34 polypeptide, where the variant A34 polypeptide comprises: i) an R91S substitution; and ii) a K151E substitution; and b) a nucleotide sequence encoding a variant A56 polypeptide, where the variant A56 polypeptide comprises an I269F substitution.

Variant B5 Polypeptides

In some embodiments, an OVV provided by the resent disclosure comprises a nucleotide sequence encoding a variant B5 polypeptide. Examples of wild-type amino acid sequences of vaccinia virus B5 polypeptides are depicted in FIG. 4D and set forth in SEQ ID Nos: 50-56. A variant B5 polypeptide can comprise 1, 2, 3, 4, or more amino acid substitutions at amino acid positions of N39, L90, N94, S197, S199, K229, V233, I236, V238, T240, N241, E243, V247, D248, G250, D263, E268, E270, D272, S273, D275, and A276, where the amino acid numbering is based on the numbering shown in FIG. 4D.

In some cases, a variant B5 polypeptide comprises a single amino acid substitution at one of N39, L90, N94, S197, S199, K229, V233, 1236, V238, T240, N241, E243, V247, D248, G250, D263, E268, E270, E272, S273, E275, and A276, where the amino acid numbering is based on the numbering shown in FIG. 4D. In some cases, a variant B5 polypeptide comprises 2 amino acid substitutions at positions selected from N39, L90, N94, S197, S199, K229, V233, 1236, V238, T240, N241, E243, V247, D248, G250, D263, E268, E270, E272, S273, E275, and A276, where the amino acid numbering is based on the numbering shown in FIG. 4D. In some cases, a variant B5 polypeptide comprises 3 amino acid substitutions at positions selected from N39, L90, N94, S197, S199, K229, V233, 1236, V238, T240, N241, E243, V247, D248, G250, D263, E268, E270, E272, S273, E275, and A276, where the amino acid numbering is based on the numbering shown in FIG. 4D. In some cases, a variant B5 polypeptide comprises 4 amino acid substitutions at positions selected from N39, L90, N94, S197, S199, K229, V233, 1236, V238, T240, N241, E243, V247, D248, G250, D263, E268, E270, E272, S273, E275, and A276, where the amino acid numbering is based on the numbering shown in FIG. 4D. In some cases, a variant B5 polypeptide comprises 5 amino acid substitutions at positions selected from N39, L90, N94, S197, S199, K229, V233, 1236, V238, T240, N241, E243, V247, D248, G250, D263, E268, E270, E272, S273, E275, and A276, where the amino acid numbering is based on the numbering shown in FIG. 4D. In some cases, a variant B5 polypeptide comprises 6 amino acid substitutions at positions selected from N39, L90, N94, S197, S199, K229, V233, 1236, V238, T240, N241, E243, V247, D248, G250, D263, E268, E270, E272, S273, E275, and A276, where the amino acid numbering is based on the numbering shown in FIG. 4D.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 39 other than Asn. For example, in some cases, a variant B5 polypeptide comprises Ala, Gly, lie, Leu, Pro, Val, Phe, Trp, Tyr, Asp, Glu, Arg, His, Lys, Ser, Thr, Cys, Met, or Gln at position 39, based on the amino acid numbering depicted in FIG. 4D. In some cases, a variant B5 polypeptide comprises an N39G substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 90 other than Leu. For example, in some cases, a variant B5 polypeptide comprises Ala, Gly, lie, Pro, Val, Phe, Trp, Tyr, Asp, Glu, Arg, His, Lys, Ser, Thr, Cys, Met, Asn, or Gln at position 90, based on the amino acid numbering depicted in FIG. 4D. In some cases, a variant B5 polypeptide comprises an L90R substitution. In some cases, a variant B5 polypeptide comprises an L90H substitution. In some cases, a variant B5 polypeptide comprises an L90K substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 94 other than Asn. For example, in some cases, a variant B5 polypeptide comprises Ala, Gly, lie, Leu, Pro, Val, Phe, Trp, Tyr, Asp, Glu, Arg, His, Lys, Ser, Thr, Cys, Met, or Gln at position 94, based on the amino acid numbering depicted in FIG. 4D. In some cases, a variant B5 polypeptide comprises an N94T substitution. In some cases, a variant B5 polypeptide comprises an N94S substitution. In some cases, a variant B5 polypeptide comprises an N94Q substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 197 other than Ser. For example, in some cases, a variant B5 polypeptide comprises Ala, Gly, lie, Leu, Pro, Val, Phe, Trp, Tyr, Asp, Glu, Arg, His, Lys, Thr, Cys, Met, Asn, or Gln at position 197, based on the amino acid numbering depicted in FIG. 4D. In some cases, a variant B5 polypeptide comprises an S197F substitution. In some cases, a variant B5 polypeptide comprises an S197V substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 199 other than Ser. For example, in some cases, a variant B5 polypeptide comprises Ala, Gly, lie, Leu, Pro, Val, Phe, Trp, Tyr, Asp, Glu, Arg, His, Lys, Thr, Cys, Met, Asn, or Gln at position 199, based on the amino acid numbering depicted in FIG. 4D. In some cases, a variant B5 polypeptide comprises an S199M substitution. In some cases, a variant B5 polypeptide comprises an S199L substitution. In some cases, a variant B5 polypeptide comprises an S1991 substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 229 other than Lys. For example, in some cases, a variant B5 polypeptide comprises Ala, Gly, lie, Leu, Pro, Val, Phe, Trp, Tyr, Asp, Glu, Arg, His, Ser, Thr, Cys, Met, Asn, or Gln at position xx, based on the amino acid numbering depicted in FIG. 4D. In some cases, a variant B5 polypeptide comprises a K229C substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 233 other than Val. For example, in some cases, a variant B5 polypeptide comprises Ala, Gly, lie, Leu, Pro, Phe, Trp, Tyr, Asp, Glu, Arg, His, Lys, Ser, Thr, Cys, Met, Asn, or Gln at position xx, based on the amino acid numbering depicted in FIG. 4D. In some cases, a variant B5 polypeptide comprises a V233D substitution. In some cases, a variant B5 polypeptide comprises a V233E substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 236 other than lie. For example, in some cases, a variant B5 polypeptide comprises Ala, Gly, Leu, Pro, Val, Phe, Trp, Tyr, Asp, Glu, Arg, His, Lys, Ser, Thr, Cys, Met, Asn, or Gln at position 236, based on the amino acid numbering depicted in FIG. 4D. In some cases, a variant B5 polypeptide comprises an I236P substitution. In some cases, a variant B5 polypeptide comprises an I236L substitution. In some cases, a variant B5 polypeptide comprises an I236C substitution. In some cases, a variant B5 polypeptide comprises an I236V substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 238 other than Val. For example, in some cases, a variant B5 polypeptide comprises Ala, Gly, lie, Leu, Pro, Phe, Trp, Tyr, Asp, Glu, Arg, His, Lys, Ser, Thr, Cys, Met, Asn, or Gln at position 238, based on the amino acid numbering depicted in FIG. 4D. In some cases, a variant B5 polypeptide comprises a V238R substitution. In some cases, a variant B5 polypeptide comprises a V238H substitution. In some cases, a variant B5 polypeptide comprises a V238K substitution. In some cases, a variant B5 polypeptide comprises a V238W substitution. In some cases, a variant B5 polypeptide comprises a V238Y substitution. In some cases, a variant B5 polypeptide comprises a V238F substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 240 other than Thr. For example, in some cases, a variant B5 polypeptide comprises Ala, Gly, lie, Leu, Pro, Val, Phe, Trp, Tyr, Asp, Glu, Arg, His, Lys, Ser, Cys, Met, Asn, or Gln at position 240, based on the amino acid numbering depicted in FIG. 4D. In some cases, a variant B5 polypeptide comprises a T240R substitution. In some cases, a variant B5 polypeptide comprises a T240H substitution. In some cases, a variant B5 polypeptide comprises a T240K substitution. In some cases, a variant B5 polypeptide comprises a T240Y substitution. In some cases, a variant B5 polypeptide comprises a T240W substitution. In some cases, a variant B5 polypeptide comprises a T240F substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 241 other than Asn. For example, in some cases, a variant B5 polypeptide comprises Ala, Gly, lie, Leu, Pro, Val, Phe, Trp, Tyr, Asp, Glu, Arg, His, Lys, Ser, Thr, Cys, Met, or Gln at position 241, based on the amino acid numbering depicted in FIG. 4D. In some cases, a variant B5 polypeptide comprises an N241T substitution. In some cases, a variant B5 polypeptide comprises an N241S substitution. In some cases, a variant B5 polypeptide comprises an N241Q substitution. In some cases, a variant B5 polypeptide comprises an N241G substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 243 other than Glu. For example, in some cases, a variant B5 polypeptide comprises Ala, Gly, lie, Leu, Pro, Val, Phe, Trp, Tyr, Asp, Arg, His, Lys, Ser, Thr, Cys, Met, Asn, or Gln at position 243, based on the amino acid numbering depicted in FIG. 4D. In some cases, a variant B5 polypeptide comprises an E243G substitution. In some cases, a variant B5 polypeptide comprises an E243V substitution. In some cases, a variant B5 polypeptide comprises an E243A substitution. In some cases, a variant B5 polypeptide comprises an E2431 substitution. In some cases, a variant B5 polypeptide comprises an E243L substitution. In some cases, a variant B5 polypeptide comprises an E243S substitution. In some cases, a variant B5 polypeptide comprises an E243T substitution. In some cases, a variant B5 polypeptide comprises an E243R substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 247 other than Val. For example, in some cases, a variant B5 polypeptide comprises Ala, Gly, lie, Leu, Pro, Phe, Trp, Tyr, Asp, Glu, Arg, His, Lys, Ser, Thr, Cys, Met, Asn, or Gln at position 247, based on the amino acid numbering depicted in FIG. 4D. In some cases, a variant B5 polypeptide comprises a V247S substitution. In some cases, a variant B5 polypeptide comprises a V247T substitution. In some cases, a variant B5 polypeptide comprises a V247N substitution. In some cases, a variant B5 polypeptide comprises a V247Q substitution. In some cases, a variant B5 polypeptide comprises a V247W substitution. In some cases, a variant B5 polypeptide comprises a V247Y substitution. In some cases, a variant B5 polypeptide comprises a V247F substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 248 other than Asp. For example, in some cases, a variant B5 polypeptide comprises Ala, Gly, lie, Leu, Pro, Val, Phe, Trp, Tyr, Glu, Arg, His, Lys, Ser, Thr, Cys, Met, Asn, or Gln at position 248, based on the amino acid numbering depicted in FIG. 4D. In some cases, a variant B5 polypeptide comprises a D248Y substitution. In some cases, a variant B5 polypeptide comprises a D248F substitution. In some cases, a variant B5 polypeptide comprises a D248W substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 250 other than Gly. For example, in some cases, a variant B5 polypeptide comprises Ala, lie, Leu, Pro, Val, Phe, Trp, Tyr, Asp, Glu, Arg, His, Lys, Ser, Thr, Cys, Met, Asn, or Gln at position 250, based on the amino acid numbering depicted in FIG. 4D. In some cases, a variant B5 polypeptide comprises a G250A substitution. In some cases, a variant B5 polypeptide comprises a G250V substitution. In some cases, a variant B5 polypeptide comprises a G2501 substitution. In some cases, a variant B5 polypeptide comprises a G250L substitution. In some cases, a variant B5 polypeptide comprises a G250R substitution. In some cases, a variant B5 polypeptide comprises a G250H substitution. In some cases, a variant B5 polypeptide comprises a G250K substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 263 other than Asp. For example, in some cases, a variant B5 polypeptide comprises Ala, Gly, lie, Leu, Pro, Val, Phe, Trp, Tyr, Glu, Arg, His, Lys, Ser, Thr, Cys, Met, Asn, or Gln at position 263, based on the amino acid numbering depicted in FIG. 4D. In some cases, a variant B5 polypeptide comprises a D263V substitution. In some cases, a variant B5 polypeptide comprises a D263A substitution. In some cases, a variant B5 polypeptide comprises a D2631 substitution. In some cases, a variant B5 polypeptide comprises a D263L substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 268 other than Glu. For example, in some cases, a variant B5 polypeptide comprises Ala, Gly, lie, Leu, Pro, Val, Phe, Trp, Tyr, Asp, Arg, His, Lys, Ser, Thr, Cys, Met, Asn, or Gln at position 268, based on the amino acid numbering depicted in FIG. 4D. In some cases, a variant B5 polypeptide comprises an E268T substitution. In some cases, a variant B5 polypeptide comprises an E268S substitution. In some cases, a variant B5 polypeptide comprises an E268N substitution. In some cases, a variant B5 polypeptide comprises an E268Q substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 270 other than Glu. For example, in some cases, a variant B5 polypeptide comprises Ala, Gly, lie, Leu, Pro, Val, Phe, Trp, Tyr, Asp, Arg, His, Lys, Ser, Thr, Cys, Met, Asn, or Gln at position 270, based on the amino acid numbering depicted in FIG. 4D. In some cases, a variant B5 polypeptide comprises an E270S substitution. In some cases, a variant B5 polypeptide comprises an E270T substitution. In some cases, a variant B5 polypeptide comprises an E270N substitution. In some cases, a variant B5 polypeptide comprises an E270Q substitution. In some cases, a variant B5 polypeptide comprises an E270G substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 272 other than Glu. For example, in some cases, a variant B5 polypeptide comprises Ala, Gly, lie, Leu, Pro, Val, Phe, Trp, Tyr, Asp, Arg, His, Lys, Ser, Thr, Cys, Met, Asn, or Gln at position 272, based on the amino acid numbering depicted in FIG. 4D. In some cases, a variant B5 polypeptide comprises an E272G substitution. In some cases, a variant B5 polypeptide comprises an E272P substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 273 other than Ser. For example, in some cases, a variant B5 polypeptide comprises Ala, Gly, lie, Leu, Pro, Val, Phe, Trp, Tyr, Asp, Glu, Arg, His, Lys, Thr, Cys, Met, Asn, or Gln at position 273, based on the amino acid numbering depicted in FIG. 4D. In some cases, a variant B5 polypeptide comprises an S273I substitution. In some cases, a variant B5 polypeptide comprises an S273L substitution. In some cases, a variant B5 polypeptide comprises an S273V substitution. In some cases, a variant B5 polypeptide comprises an S273A substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 275 other than Glu. For example, in some cases, a variant B5 polypeptide comprises Ala, Gly, lie, Leu, Pro, Val, Phe, Trp, Tyr, Asp, Arg, His, Lys, Ser, Thr, Cys, Met, Asn, or Gln at position 275, based on the amino acid numbering depicted in FIG. 4D. In some cases, a variant B5 polypeptide comprises an E275F substitution. In some cases, a variant B5 polypeptide comprises an E275Y substitution. In some cases, a variant B5 polypeptide comprises an E275W substitution. In some cases, a variant B5 polypeptide comprises an E275S substitution. In some cases, a variant B5 polypeptide comprises an E275T substitution. In some cases, a variant B5 polypeptide comprises an E275N substitution. In some cases, a variant B5 polypeptide comprises an E275Q substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an amino acid at position 276 other than Ala. For example, in some cases, a variant B5 polypeptide comprises Gly, lie, Leu, Pro, Val, Phe, Trp, Tyr, Asp, Glu, Arg, His, Lys, Ser, Thr, Cys, Met, Asn, or Gln at position 276, based on the amino acid numbering depicted in FIG. 4D. In some cases, a variant B5 polypeptide comprises an A276F substitution. In some cases, a variant B5 polypeptide comprises an A276Y substitution. In some cases, a variant B5 polypeptide comprises an A276W substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; comprises an amino acid at position 90 other than Leu, based on the amino acid numbering depicted in FIG. 4D; and comprises an amino acid at position 273 other than Ser, based on the amino acid numbering depicted in FIG. 4D. In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an L90R substitution and an S273V substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; comprises an amino acid at position 229 other than Lys, based on the amino acid numbering depicted in FIG. 4D; and comprises an amino acid at position 273 other than Ser, based on the amino acid numbering depicted in FIG. 4D. In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises a K229C substitution and an S273L substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; comprises an amino acid at position 39 other than Asn, based on the amino acid numbering depicted in FIG. 4D; and comprises an amino acid at position 273 other than Ser, based on the amino acid numbering depicted in FIG. 4D. In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an N39G substitution and an S273I substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; comprises an amino acid at position 236 other than lie, based on the amino acid numbering depicted in FIG. 4D; and comprises an amino acid at position 238 other than Val, based on the amino acid numbering depicted in FIG. 4D. In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an I236P substitution and a V238R substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; comprises an amino acid at position 233 other than Val, based on the amino acid numbering depicted in FIG. 4D; and comprises an amino acid at position 236 other than lie, based on the amino acid numbering depicted in FIG. 4D. In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises a V233D substitution and an I236L substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; comprises an amino acid at position 263 other than Asp, based on the amino acid numbering depicted in FIG. 4D; and comprises an amino acid at position 268 other than Glu, based on the amino acid numbering depicted in FIG. 4D. In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises a D263V substitution and an E268T substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; comprises an amino acid at position 263 other than Asp, based on the amino acid numbering depicted in FIG. 4D; comprises an amino acid at position 270 other than Glu, based on the amino acid numbering depicted in FIG. 4D; comprises an amino acid at position 272 other than Glu, based on the amino acid numbering depicted in FIG. 4D; and comprises an amino acid at position 275 other than Glu, based on the amino acid numbering depicted in FIG. 4D. In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises a D263A substitution, an E270S substitution, an E272G substitution, and an E275F substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; comprises an amino acid at position 236 other than lie, based on the amino acid numbering depicted in FIG. 4D; comprises an amino acid at position 238 other than Val, based on the amino acid numbering depicted in FIG. 4D; comprises an amino acid at position 240 other than Thr, based on the amino acid numbering depicted in FIG. 4D; and comprises an amino acid at position 243 other than Glu, based on the amino acid numbering depicted in FIG. 4D. In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an I236P substitution, a V238R substitution, a T240R substitution, and an E243G substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; comprises an amino acid at position 233 other than Val, based on the amino acid numbering depicted in FIG. 4D; comprises an amino acid at position 236 other than lie, based on the amino acid numbering depicted in FIG. 4D; comprises an amino acid at position 238 other than Val, based on the amino acid numbering depicted in FIG. 4D; comprises an amino acid at position 240 other than Thr, based on the amino acid numbering depicted in FIG. 4D; and comprises an amino acid at position 243 other than Glu, based on the amino acid numbering depicted in FIG. 4D. In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises a V233D substitution, an I236L substitution, a V238W substitution, a T240Y substitution, and an E243R substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; comprises an amino acid at position 241 other than Asn, based on the amino acid numbering depicted in FIG. 4D; comprises an amino acid at position 243 other than Glu, based on the amino acid numbering depicted in FIG. 4D; comprises an amino acid at position 247 other than Val, based on the amino acid numbering depicted in FIG. 4D; comprises an amino acid at position 250 other than Gly, based on the amino acid numbering depicted in FIG. 4D; and comprises an amino acid at position 276 other than Ala, based on the amino acid numbering depicted in FIG. 4D. In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an N241T substitution, an E243V substitution, a V247S substitution, a G250R substitution, and an A276F substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; comprises an amino acid at position 263 other than Asp, based on the amino acid numbering depicted in FIG. 4D; comprises an amino acid at position 268 other than Glu, based on the amino acid numbering depicted in FIG. 4D; comprises an amino acid at position 270 other than Glu, based on the amino acid numbering depicted in FIG. 4D; comprises an amino acid at position 272 other than Glu, based on the amino acid numbering depicted in FIG. 4D; and comprises an amino acid at position 275 other than Glu, based on the amino acid numbering depicted in FIG. 4D. In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises a D263V substitution, an E268T substitution, an E270G substitution, an E272P substitution, and an E275S substitution.

In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; comprises an amino acid at position 241 other than Asn, based on the amino acid numbering depicted in FIG. 4D; comprises an amino acid at position 243 other than Glu, based on the amino acid numbering depicted in FIG. 4D; comprises an amino acid at position 247 other than Val, based on the amino acid numbering depicted in FIG. 4D; comprises an amino acid at position 248 other than Asp, based on the amino acid numbering depicted in FIG. 4D; comprises an amino acid at position 250 other than Gly, based on the amino acid numbering depicted in FIG. 4D; and comprises an amino acid at position 276 other than Ala, based on the amino acid numbering depicted in FIG. 4D. In some cases, a variant B5 polypeptide comprises an amino acid sequence having at least 95%, at least 96%, at least 97%, at least 98%, or at least 99%, amino acid sequence identity to a B5 amino acid sequence depicted in FIG. 4D; and comprises an N241G substitution, an E243S substitution, a V247W substitution, a D248Y substitution, a G250A substitution, and an A276F substitution.

In any one of the above-described embodiments, the replication-competent, recombinant oncolytic vaccinia virus can further include a nucleotide sequence encoding a variant A34 polypeptide, where the variant A34 polypeptide has a K151E substitution. For example, the variant A34 polypeptide can comprise an amino acid sequence having at least 95%, at least 96%, at least 97%, at least 98%, or at least 99%, amino acid sequence identity to an A34 amino acid sequence depicted in FIG. 2C; and comprises a K151E substitution. Non-limiting examples of combinations are set out in Table 2.

Variant A34 Polypeptides and Variant B5 Polypeptides

In some cases, an OVV of the present disclosure comprises nucleotide sequences encoding both a variant A34 polypeptide and a variant B5 polypeptide; such an OVV can comprise nucleotide sequences encoding any of the above-described variant A34 polypeptides and any of the above-described B5 polypeptides in any combination. Non-limiting examples of combinations are set out in Table 2.

As one non-limiting example, an OVV of the present disclosure comprises: a) a nucleotide sequence encoding a variant A34 polypeptide, where the variant A34 polypeptide comprises an F94H substitution; and b) a nucleotide sequence encoding a variant B5 polypeptide, where the variant B5 polypeptide comprises an N39G substitution and an S273I substitution. As another non-limiting example, an OVV of the present disclosure comprises: a) a nucleotide sequence encoding a variant A34 polypeptide, where the variant A34 polypeptide comprises an F94H substitution and a K151E substitution; and b) a nucleotide sequence encoding a variant B5 polypeptide, where the variant B5 polypeptide comprises an N39G substitution and an S273I substitution.

Construction of the Oncolytic Vaccinia Viruses

An OVV of the present disclosure can be constructed from any of a variety of strains of vaccinia virus. Examples of vaccinia virus strain suitable for use include, but not limited to, the strains Lister, New York City Board of Health (NYBH), Wyeth, Copenhagen, Western Reserve (WR), Modified Vaccinia Ankara (MVA), EM63, Ikeda, Dalian, LIVP, Tian Tan, IHD-J, Tashkent, Bern, Paris, Dairen and derivatives the like. In some cases, an OVV of the present disclosure is a Copenhagen strain vaccinia virus. In some cases, an OVV of the present disclosure is a WR strain vaccinia virus.

The nucleotide sequences of the genomes of vaccinia viruses of various strains are known in the art. See, e.g., Goebel et al. (1990) Virology 179:247; Goebel et al. (1990) Virology 179:517. The nucleotide sequence of the Copenhagen strain vaccinia virus is known; see, e.g., GenBank Accession No. M35027. The nucleotide sequence of the WR strain vaccinia virus is known; see, e.g., GenBank Accession No. AY243312; and GenBank Accession No. NC_006998. The WR strain of vaccinia virus is available from the American Type Culture Collection (ATCC); ATCC VR-1354.

The vaccinia virus used to construct an OVV of the present disclosure can include attenuated and/or tumor-selective vaccinia viruses. As used herein, “attenuated” means low toxicity (for example, low virus replication, low cytolytic activity, low cytotoxic activity) to normal cells (for example, non-tumor cells). As used herein, “tumor selective” means toxicity to tumor cells (for example, oncolytic) higher than that to normal cells (for example, non-tumor cell). Vaccinia viruses genetically modified to be deficient in the function of a specific protein or to suppress the expression of a specific gene or protein (Guse et al. (2011) Expert Opinion on Biological Therapy 11:595) may be used in an oncolytic virus of the present disclosure. For example, in order to increase tumor selectivity of vaccinia virus, vaccinia virus deficient in the function of vaccinia growth factor (VGF) (McCart et al. (2001) Cancer Research 61:8751); vaccinia virus having a modified vaccinia virus TK gene, a modified hemagglutinin (HA) gene, and a modified F3 gene or an interrupted F3 locus (WO 2005/047458), vaccinia virus deficient in the function of VGF and O1L (WO 2015/076422); vaccinia virus in which a target sequence of a microRNA whose expression is decreased in cancer cells is inserted into the 3′ noncoding region of the B5R gene (WO 2011/125469); vaccinia virus deficient in the function of HA and F14.5L (Zhang et al. (2007) Cancer Research 67:10038); vaccinia virus deficient in the function of ribonucleotide reductase (Gammon et al. (2010) PLoS Pathogens 6:e1000984); vaccinia virus deficient in the function of serine protease inhibitor (e.g., SPI-1, SPI-2) (Guo et al. (2005) Cancer Research 65:9991); vaccinia virus deficient in the function of SPI-1 and SPI-2 (Yang et al. (2007) Gene Therapy 14:638); vaccinia virus deficient in the function of ribonucleotide reductase genes F4L or 14L (Child et al. (1990) Virology 174:625; Potts et al. (2017) EMBO Mol. Med. 9:638); vaccinia virus deficient in the function of B18R (B19R in Copenhagen strain) (Symons et al. (1995) Cell 81:551; Kirn et al. (2007) PLoS Medicine 4:e353); vaccinia virus deficient in the function of A48R (Hughes et al. (1991) J. Biol. Chem. 266:20103); vaccinia virus deficient in the function of B8R (Verardi et al. (2001) J. Virol. 75:11); vaccinia virus deficient in the function of B15R (B16R in Copenhagen strain) (Spriggs et al. (1992) Cell 71:145); vaccinia virus deficient in the function of A41R (Ng et al. (2001) Journal of General Virology 82:2095); vaccinia virus deficient in the function of A52R (Bowie et al. (2000) Proc. Natl. Acad. Sci. USA 97:10162); vaccinia virus deficient in the function of F1L (Gerlic et al. (2013) Proc. Natl. Acad. Sci. USA 110:7808); vaccinia virus deficient in the function of E3L (Chang et al. (1992) Proc. Natl. Acad. Sci. USA 89:4825); vaccinia virus deficient in the function of A44R-A46R (Bowie et al. (2000) Proc. Natl. Acad. Sci. USA 97:10162); vaccinia virus deficient in the function of K1L (Bravo Cruz et al. (2017) Journal of Virology 91:e00524); vaccinia virus deficient in the function of A48R, B18R, C11R, and TK (Mejías-Pérez et al. (2017) Molecular 25 Therapy: Oncolytics 8:27); or vaccinia virus having mutations in the E3L and K3L regions (WO 2005/007824) may be used. Moreover, vaccinia virus deficient in the function of O1 L may be used (Schweneker et al. (2012) J. Virol. 86:2323). Moreover, vaccinia virus deficient in the extracellular region of B5R (Bell et al. (2004) Virology 325:425) or vaccinia virus deficient in the A34R region (Thirunavukarasu et al. (2013) Molecular Therapy 21:1024) may be used. Moreover, vaccinia virus deficient in interleukin-1b (IL-1b) receptor (WO 2005/030971) may be used. Such insertion of a foreign gene or deletion or mutation of a gene can be made, for example, by a known homologous recombination or site-directed mutagenesis. Moreover, vaccinia virus having a combination of two or more of such genetic modifications may be used in an OVV of the present disclosure.

As used herein, “being deficient” means that the gene region, or a gene product, specified by this term has reduced or no function and includes a deficiency resulting from one or more of: i) mutation (e.g., substitution, inversion, etc.) and/or truncation and/or deletion of the gene region; ii) mutation and/or truncation and/or deletion of a promoter region controlling expression of the gene region; and iii) mutation and/or truncation and/or deletion of a polyadenylation sequence such that translation of a polypeptide encoded by the gene region is reduced or eliminated. An OVV of the present disclosure that comprises a genetic alteration such that the replication-competent, recombinant oncolytic vaccinia virus is “deficient” in a given vaccinia virus gene exhibits reduced production and/or activity of a gene product (e.g., mRNA gene product; polypeptide gene product) of the gene; for example, the amount and/or activity of the gene product is less than 75%, less than 60%, less than 50%, less than 40%, less than 30%, less than 25%, less than 20%, less than 15%, less than 10%, less than 5%, or less than 1% of the amount and/or activity of the same gene product produced by wild-type vaccinia virus, or by a control vaccinia virus that does not comprise the genetic alteration. For example, “being deficient” may be a result of the deletion in a region consisting of the specified gene region or the deletion in a neighboring gene region comprising the specified gene region. As an example, a mutation and/or truncation and/or deletion of a promoter region that reduces transcription of a gene region can result in deficiency. A gene region can also be rendered deficient through incorporation of a transcriptional termination element such that translation of a polypeptide encoded by the gene region is reduced or eliminated. A gene region can also be rendered deficient through use of a gene-editing enzyme or a gene-editing complex to reduce or eliminate transcription of the gene region. A gene region can also be rendered deficient through use of competitive reverse promoter/polymerase occupancy to reduce or eliminate transcription of the gene region. A gene region can also be rendered deficient by insertion of a nucleic acid into the gene region, thereby knocking out the gene region.

In some embodiments, an OVV provided by the present disclosure is vaccinia virus thymidine kinase (TK) deficient. In some cases, an OVV of the present disclosure comprises a deletion of all or a portion of the vaccinia virus TK coding region, such that the replication-competent, recombinant oncolytic vaccinia virus is TK deficient. For example, in some cases, an OVV of the present disclosure comprises a deletion in the J2R gene (i.e., gene that encodes viral thymidine kinase). See, e.g., Mejía-Perez et al. (2018) Mol. Ther. Oncolytics 8:27. In some cases, an OVV of the present disclosure comprises an insertion into the J2R region, thereby resulting in reduced vaccinia virus TK expression or activity.

An OVV of the present disclosure will in some instances comprise an A34R gene encoded an A34 polypeptide having a K151E substitution (i.e., comprising a modification that provides for a K151E substitution in the encoded polypeptide). See, e.g., Blasco et al. (1993) J. Virol. 67(6):3319-3325; and Thirunavukarasu et al. (2013) Mol. Ther. 21:1024. The A34R gene encodes vaccinia virus gp22-24.

In some cases, an OVV of the present disclosure comprises a heterologous nucleic acid comprising a nucleotide sequence encoding an immunomodulatory polypeptide (a heterologous immunomodulatory polypeptide). Examples of immunomodulatory polypeptide include cytokines (such as IL-2, and IL-12), granulocyte-macrophage colony stimulating factor (GM-CSF), and tumor necrosis factor-alpha (TNF-α).

An OVV of the present disclosure exhibits oncolytic activity. Examples of methods for evaluating whether a given virus exhibits oncolytic activity include a method for evaluating decrease of the survival rate of cancer cells by the addition of the virus. Examples of cancer cells to be used for the evaluation include the malignant melanoma cell RPMI-7951 (for example, ATCC HTB-66), the lung adenocarcinoma HCC4006 (for example, ATCC CRL-2871), the lung carcinoma A549 (for example, ATCC CCL-185), the lung carcinoma HOP-62 (for example, DCTD Tumor Repository), the lung carcinoma EKVX (for example, DCTD Tumor Repository), the small cell lung cancer cell DMS 53 (for example, ATCC CRL-2062), the lung squamous cell carcinoma NCI-H226 (for example, ATCC CRL-5826), the kidney cancer cell Caki-1 (for example, ATCC HTB-46), the bladder cancer cell 647-V (for example, DSMZ ACC 414), the head and neck cancer cell Detroit 562 (for example, ATCC CCL-138), the breast cancer cell JIMT-1 (for example, DSMZ ACC 589), the breast cancer cell MDA-MB-231 (for example, ATCC HTB-26), the breast cancer cell MCF7 (for example, ATCC HTB-22), the breast cancer HS-578T (for example, ATCC HTB-126), the breast ductal carcinoma T-47D (for example, ATCC HTB-133), the esophageal cancer cell OE33 (for example, ECACC 96070808), the glioblastoma U-87MG (for example, ECACC 89081402), the neuroblastoma GOTO (for example, JCRB JCRB0612), the myeloma RPMI 8226 (for example, ATCC CCL-155), the ovarian cancer cell SK-OV-3 (for example, ATCC HTB-77), the ovarian cancer cell OVMANA (for example, JCRB JCRB1045), the cervical cancer HeLa (for example, ATCC CCL-2), the colon cancer cell RKO (for example, ATCC CRL-2577), the colon cancer cell HT-29 (for example, ATCC HTB-38), the colon cancer Colo 205 (for example, ATCC CCL-222), the colon cancer SW620 (for example, ATCC CCL-227), the colorectal carcinoma HCT 116 (for example, ATCC CCL-247), the pancreatic cancer cell BxPC-3 (for example, ATCC CRL-1687), the bone osteosarcoma U-2 OS (for example, ATCC HTB-96), the prostate cancer cell LNCaP clone FGC (for example, ATCC CRL-1740), the hepatocellular carcinoma JHH-4 (for example, JCRB JCRB0435), the mesothelioma NCI-H28 (for example, ATCC CRL-5820), the cervical cancer cell SiHa (for example, ATCC HTB-35), and the gastric cancer cell Kato III (for example, RIKEN BRC RCB2088). Also suitable for use are cancer cells obtained from a patient. For example, colorectal cancer cells, breast cancer cells, or non-small lung cancer cells obtained from a patient can be used.

As noted above, an OVV of the present disclosure exhibits enhanced virus spreading and/or enhanced EEV production, compared to a control vaccinia virus that does not include a) a nucleotide sequence encoding a variant A33 polypeptide, as described herein (e.g., compared to a control vaccinia virus that comprises a nucleotide sequence encoding a wild-type A33 polypeptide, such as an A33 polypeptide having an amino acid sequence depicted in FIG. 1C); and/or b) a nucleotide sequence encoding a variant A34 polypeptide, as described herein (e.g., compared to a control vaccinia virus that comprises a nucleotide sequence encoding a wild-type A34 polypeptide, such as an A34 polypeptide having an amino acid sequence depicted in FIG. 2C); and/or c) a variant B5 polypeptide, as described herein (e.g., compared to a control vaccinia virus that comprises a nucleotide sequence encoding a wild-type B5 polypeptide, such as a B5 polypeptide having an amino acid sequence depicted in FIG. 4D).

Compositions

The present disclosure provides compositions comprising an OVV of the present disclosure. In some cases, the composition is a pharmaceutical composition. A pharmaceutical composition comprising an OVV of the present disclosure can further comprises a pharmaceutically acceptable carrier(s). As used herein, the terms “pharmacologically acceptable carrier” and “pharmacologically acceptable excipient” are used interchangeably and refer to any substance that is suitable for use in the formulation of the OVV for administration to a human. Such a carrier generally is mixed with an OVV of the present disclosure, and can be a solid, semi-solid, or liquid agent. The composition can be a solution or a suspension in the desired carrier or diluent. Any of a variety of pharmaceutically acceptable carriers can be used including, without limitation, aqueous media such as, e.g., distilled, deionized water, saline; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with an OVV of the present disclosure, its use in pharmaceutically acceptable compositions is contemplated. Non-limiting examples of specific uses of such pharmaceutical carriers can be found in “Pharmaceutical Dosage Forms and Drug Delivery Systems” (Howard C. Ansel et al., eds., Lippincott Williams & Wilkins Publishers, 7^{th} ed. 1999); “Remington: The Science and Practice of Pharmacy” (Alfonso R. Gennaro ed., Lippincott, Williams & Wilkins, 20^{th} 2000); “Goodman & Gilman's The Pharmacological Basis of Therapeutics” Joel G. Hardman et al., eds., McGraw-Hill Professional, 10^{th} ed. 2001); and “Handbook of Pharmaceutical Excipients” (Raymond C. Rowe et al., APhA Publications, 4^{th} edition 2003).

A subject pharmaceutical composition can optionally include, without limitation, other pharmaceutically acceptable components, including, without limitation, buffers, preservatives, tonicity adjusters, salts, antioxidants, physiological substances, pharmacological substances, bulking agents, emulsifying agents, wetting agents, and the like. Various buffers and means for adjusting pH can be used to prepare a pharmaceutical composition disclosed in the present specification, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate and a stabilized oxy chloro composition, for example, PURITE™. Tonicity adjustors suitable for inclusion in a subject pharmaceutical composition include, without limitation, salts such as, e.g., sodium chloride, potassium chloride, mannitol or glycerin and other pharmaceutically acceptable tonicity adjustor. It is understood that these and other substances known in the art of pharmacology can be included in a subject pharmaceutical composition.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

A pharmaceutical composition of the present disclosure can comprise an OVV of the present disclosure in an amount of from about 10² plaque-forming units (pfu) per ml (pfu/ml) to about 10⁴ pfu/ml, from about 10⁴ pfu/ml to about 10⁵ pfu/ml, from about 10⁵ pfu/ml to about 10⁶ pfu/ml, from about 10⁶ pfu/ml to about 10⁷ pfu/ml, from about 10⁷ pfu/ml to about 10⁸ pfu/ml, from about 10⁸ pfu/ml to about 10⁹ pfu/ml, from about 10⁹ pfu/ml to about 10¹⁰ pfu/ml, from about 10¹⁰ pfu/ml to about 10¹¹ pfu/ml, or from about 10¹¹ pfu/ml to about 10¹² pfu/ml.

Methods of Inducing Oncolysis

The present disclosure provides methods of inducing oncolysis in an individual having a tumor, the methods comprising administering to the individual an effective amount of an OVV of the present disclosure or a composition of the present disclosure. Administration of an effective amount of an OVV of the present disclosure, or a composition of the present disclosure, is also referred to herein as “virotherapy.” The present disclosure further provides an OVV or composition of the present disclosure for use in methods of inducing oncolysis in an individual having a tumor. The disclosure further provides the use of an OVV or composition of the present disclosure in the manufacture of a medicament for use in such methods.

In some cases, an “effective amount” of an OVV of the present disclosure is an amount that, when administered in 1, 2, or more doses to an individual in need thereof, reduces the number of cancer cells in the individual. For example, in some cases, an “effective amount” of an OVV of the present disclosure is an amount that, when administered in one or more doses to an individual in need thereof, reduces the number of cancer cells in the individual by at least 10%, at least 15%, at least 20%, at least 25%, at least 30%, at least 40%, at least 50%, at least 60%, at least 70%, at least 80%, at least 90%, or at least 95%, compared to the number of cancer cells in the individual before administration of the replication-competent, recombinant oncolytic vaccinia virus, or in the absence of administration with the replication-competent, recombinant oncolytic vaccinia virus. In some cases, an “effective amount” of an OVV of the present disclosure is an amount that, when administered in one or more doses to an individual in need thereof, reduces the number of cancer cells in the individual to undetectable levels. In some cases, an “effective amount” of an OVV of the present disclosure is an amount that, when administered in one or more doses to an individual in need thereof, reduces the tumor mass in the individual.

For example, in some cases, an “effective amount” of an OVV of the present disclosure is an amount that, when administered in one or more doses to an individual in need thereof, reduces the tumor mass in the individual by at least 10%, at least 15%, at least 20%, at least 25%, at least 30%, at least 40%, at least 50%, at least 60%, at least 70%, at least 80%, at least 90%, or at least 95%, compared to the tumor mass in the individual before administration of the replication-competent, recombinant oncolytic vaccinia virus, or in the absence of administration with the replication-competent, recombinant oncolytic vaccinia virus.

In some cases, an “effective amount” of an OVV of the present disclosure is an amount that, when administered in one or more doses to an individual in need thereof, increases survival time of the individual. For example, in some cases, an “effective amount” of an OVV of the present disclosure is an amount that, when administered in one or more doses to an individual in need thereof, increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of administration with the replication-competent, recombinant oncolytic vaccinia virus.

In some cases, an “effective amount” of an OVV of the present disclosure is an amount that, when administered in one or more doses to an individual in need thereof, induces a durable anti-tumor immune response, e.g., an anti-tumor immune response that provides for reduction in tumor cell number and/or tumor mass and/or tumor growth for at least 1 month, at least 2 months, at least 6 months, or at least 1 year.

A suitable dosage can be determined by an attending physician or other qualified medical personnel, based on various clinical factors. As is well known in the medical arts, dosages for any one patient depend upon many factors, including the patient's size, body surface area, age, tumor burden, and other relevant factors.

An OVV of the present disclosure can be administered in an amount of from about 10² plaque-forming units (pfu) to about 10⁴ pfu, from about 10⁴ pfu to about 10⁵ pfu, from about 10⁵ pfu to about 10⁶ pfu, from about 10⁶ pfu to about 10⁷ pfu, from about 10⁷ pfu to about 10⁸ pfu, from about 10⁸ pfu to about 10⁹ pfu, from about 109 pfu to about 10¹⁰ pfu, from about 10¹⁰ pfu to about 10¹¹ pfu, or from about 10¹¹ pfu to about 10¹² pfu per dose.

In some cases, an OVV of the present disclosure is administered in a total amount of from about 1×10⁹ pfu to 5×10¹² pfu. In some cases, an OVV of the present disclosure is administered in a total amount of from about 1×10⁹ pfu to about 5×10⁹ pfu, from about 5×10⁹ pfu to about 10¹⁰ pfu, from about 10¹⁰ pfu to about 5×10¹⁰ pfu, from about 5×10¹⁰ pfu to about 10¹¹ pfu, from about 10¹¹ pfu to about 5×10¹¹ pfu, from about 5×10¹¹ pfu to about 10¹² pfu, or from about 10¹² pfu to about 5×10¹² pfu. In some cases, an OVV of the present disclosure is administered in a total amount of about 2×10¹⁰ pfu.

In some cases, an OVV of the present disclosure is administered in an amount of from about 1×10⁸ pfu/kg patient weight to about 1×10¹⁰ pfu/kg patient weight. In some cases, an OVV of the present disclosure is administered in an amount of from about 1×10⁸ pfu/kg patient weight to about 5×10⁸ pfu/kg patient weight, from about 5×10⁸ pfu/kg patient weight to about 10⁹ pfu/kg patient weight, from about 10⁹ pfu/kg patient weight to about 5×10⁹ pfu/kg patient weight, or from about 5×10⁹ pfu/kg patient weight to about 10¹⁰ pfu/kg patient weight. In some cases, an OVV of the present disclosure is administered in an amount of 1×10⁸ pfu/kg patient weight. In some cases, an OVV of the present disclosure is administered in an amount of 2×10⁸ pfu/kg patient weight. In some cases, an OVV of the present disclosure is administered in an amount of 3×10⁸ pfu/kg patient weight. In some cases, an OVV of the present disclosure is administered in an amount of 4×10⁸ pfu/kg patient weight. In some cases, an OVV of the present disclosure is administered in an amount of 5×10⁸ pfu/kg patient weight. In some cases, an OVV of the present disclosure is administered in an amount of 10⁹ pfu/kg patient weight. In some cases, an OVV of the present disclosure is administered in an amount of 5×10⁹ pfu/kg patient weight.

In some cases, multiple doses of an OVV of the present disclosure are administered. The frequency of administration of an OVV of the present disclosure can vary depending on any of a variety of factors, e.g., severity of the symptoms, etc. For example, in some embodiments, an OVV of the present disclosure is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of an OVV of the present disclosure, e.g., the period of time over which a multimeric polypeptide of the present disclosure, an OVV of the present disclosure is administered, can vary, depending on any of a variety of factors, e.g., patient response, etc. For example, an OVV of the present disclosure can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

An OVV of the present disclosure is administered to an individual using any available method and route suitable for drug delivery, including in vivo and ex vivo methods, as well as systemic and localized routes of administration.

Conventional and pharmaceutically acceptable routes of administration include intratumoral, peritumoral, intramuscular, intratracheal, intrathecal, intracranial, subcutaneous, intradermal, topical application, intravenous, intraarterial, intraperitoneal, intrabladder, rectal, nasal, oral, and other enteral and parenteral routes of administration. Routes of administration may be combined, if desired, or adjusted depending upon the replication-competent, recombinant oncolytic vaccinia virus and/or the desired effect. An OVV of the present disclosure can be administered in a single dose or in multiple doses.

In some cases, an OVV of the present disclosure is administered intravenously. In some cases, an OVV of the present disclosure is administered intramuscularly. In some cases, an OVV of the present disclosure is administered locally. In some cases, an OVV of the present disclosure is administered intratumorally. In some cases, an OVV of the present disclosure is administered peritumorally. In some cases, an OVV of the present disclosure is administered intracranially. In some cases, an OVV of the present disclosure is administered subcutaneously. In some cases, an OVV of the present disclosure is administered intra-arterially. In some cases, an OVV of the present disclosure is administered intraperitoneally. In some cases, an OVV of the present disclosure is administered via an intrabladder route of administration. In some cases, an OVV of the present disclosure is administered intrathecally.

Combinations

In some cases, an OVV of the present disclosure is administered in combination with another cancer therapy, such as surgery (e.g., surgical removal of cancerous tissue), radiation therapy, bone marrow transplantation, chemotherapeutic treatment, antibody treatment, biological response modifier treatment, immunotherapy treatment, and certain combinations of the foregoing. In some cases, a method of the present disclosure comprises: a) administering to an individual in need thereof an OVV of the present disclosure, or a composition comprising same; and b) administering to the individual a second cancer therapy. In some cases, the second cancer therapy is selected from chemotherapy, biological therapy, radiotherapy, immunotherapy, hormone therapy, anti-vascular therapy, cryotherapy, toxin therapy, oncolytic virus therapy (e.g., an oncolytic virus other than an OVV of the present disclosure), a cell therapy, and surgery.

Radiation therapy includes, but is not limited to, x-rays or gamma rays that are delivered from either an externally applied source such as a beam, or by implantation of small radioactive sources.

Suitable antibodies for use in cancer treatment include, but are not limited to, e.g., trastuzumab (Herceptin), bevacizumab (Avastin™), cetuximab (Erbitux™) panitumumab (Vectibix™), Ipilimumab (Yervoy™), rituximab (Rituxan), alemtuzumab (Lemtrada™), Ofatumumab (Arzerra™), Oregovomab (OvaRex™), Lambrolizumab (MK-3475), pertuzumab (Perjeta™), ranibizumab (Lucentis™) etc., and conjugated antibodies, e.g., gemtuzumab ozogamicin (Mylortarg™), Brentuximab vedotin (Adcetris™), ⁹⁰Y-labelled ibritumomab tiuxetan (Zevalin™)¹³¹1-labelled tositumoma (Bexxar™), etc. Suitable antibodies for use in cancer treatment include, but are not limited to, e.g., Ipilimumab targeting CTLA-4 (as used in the treatment of Melanoma, Prostate Cancer, RCC); Tremelimumab targeting CTLA-4 (as used in the treatment of CRC, Gastric, Melanoma, NSCLC); Nivolumab targeting PD-1 (as used in the treatment of Melanoma, NSCLC, RCC); MK-3475 targeting PD-1 (as used in the treatment of Melanoma); Pidilizumab targeting PD-1 (as used in the treatment of Hematologic Malignancies); BMS-936559 targeting PD-L1 (as used in the treatment of Melanoma, NSCLC, Ovarian, RCC); MED14736 targeting PD-L1; MPDL33280A targeting PD-L1 (as used in the treatment of Melanoma); Rituximab targeting CD20 (as used in the treatment of Non-Hodgkin's lymphoma); Ibritumomab tiuxetan and tositumomab (as used in the treatment of Lymphoma); Brentuximab vedotin targeting CD30 (as used in the treatment of Hodgkin's lymphoma); Gemtuzumab ozogamicin targeting CD33 (as used in the treatment of Acute myelogenous leukaemia); Alemtuzumab targeting CD52 (as used in the treatment of Chronic lymphocytic leukaemia); IGN101 and adecatumumab targeting EpCAM (as used in the treatment of Epithelial tumors (breast, colon and lung)); Labetuzumab targeting CEA (as used in the treatment of Breast, colon and lung tumors); huA33 targeting gpA33 (as used in the treatment of Colorectal carcinoma); Pemtumomab and oregovomab targeting Mucins (as used in the treatment of Breast, colon, lung and ovarian tumors); CC49 (minretumomab) targeting TAG-72 (as used in the treatment of Breast, colon and lung tumors); cG250 targeting CAIX (as used in the treatment of Renal cell carcinoma); J591 targeting PSMA (as used in the treatment of Prostate carcinoma); MOv18 and MORAb-003 (farletuzumab) targeting Folate-binding protein (as used in the treatment of Ovarian tumors); 3F8, ch14.18 and KW-2871 targeting Gangliosides (such as GD2, GD3 and GM2) (as used in the treatment of Neuroectodermal tumors and some epithelial tumors); hu3S193 and IgN311 targeting Le y (as used in the treatment of Breast, colon, lung and prostate tumors); Bevacizumab targeting VEGF (as used in the treatment of Tumor vasculature); IM-2C6 and CDP791 targeting VEGFR (as used in the treatment of Epithelium-derived solid tumors); Etaracizumab targeting Integrin_V_3 (as used in the treatment of Tumor vasculature); Volociximab targeting Integrin_5_1 (as used in the treatment of Tumor vasculature); Cetuximab, panitumumab, nimotuzumab and 806 targeting EGFR (as used in the treatment of Glioma, lung, breast, colon, and head and neck tumors); Trastuzumab and pertuzumab targeting ERBB2 (as used in the treatment of Breast, colon, lung, ovarian and prostate tumors); MM-121 targeting ERBB3 (as used in the treatment of Breast, colon, lung, ovarian and prostate, tumors); AMG 102, METMAB and SCH 900105 targeting MET (as used in the treatment of Breast, ovary and lung tumors); AVE1642, IMC-A12, MK-0646, R1507 and CP 751871 targeting IGF1R (as used in the treatment of Glioma, lung, breast, head and neck, prostate and thyroid cancer); KB004 and IIIA4 targeting EPHA3 (as used in the treatment of Lung, kidney and colon tumors, melanoma, glioma and hematological malignancies); Mapatumumab (HGS-ETR1) targeting TRAILR1 (as used in the treatment of Colon, lung and pancreas tumors and hematological malignancies); HGS-ETR2 and CS-1008 targeting TRAILR2; Denosumab targeting RANKL (as used in the treatment of Prostate cancer and bone metastases); Sibrotuzumab and F19 targeting FAP (as used in the treatment of Colon, breast, lung, pancreas, and head and neck tumors); 81C6 targeting Tenascin (as used in the treatment of Glioma, breast and prostate tumors); Blinatumomab (Blincyto; Amgen) targeting CD3 (as used in the treatment of ALL); pembrolizumab targeting PD-1 as used in cancer immunotherapy; 9E10 antibody targeting c-Myc; and the like.

In some cases, a method of the present disclosure comprises administering: a) an effective amount of an OVV of the present disclosure; and b) an anti-PD-1 antibody. In some cases, a method of the present disclosure comprises administering: a) an effective amount of an OVV of the present disclosure; and b) an anti-PD-L1 antibody. Examples of anti-PD-1 and anti-PD-L1 antibodies that may useful in the combination therapy of the present disclosure include, but are not limited to, pembrolizumab (Keytruda®; MK-3475), Nivolumab (Opdivo®; BMS-926558; MDX1106), Pidilizumab (CT-011), AMP-224, AMP-514 (MEDI-0680), PDR001, and PF-06801591 (also known as sasanlimab or RN888), BMS-936559 (MDX1105), durvalumab (MED14736; IMFINZI®), Atezolizumab (MPDL33280A; TECENTRIQ®), MSB0010718C, BCD-100 (BIOCAD Biopharmaceutical Company), tislelizumab (BGB-A317, BeiGene Ltd./Celgene Corporation), genolimzumab (CBT-501, CBT Pharmaceuticals), CBT-502 (CBT Pharmaceuticals), GLS-010 (Harbin Gloria Pharmaceuticals Co., Ltd.), sintilimab (IB1308, Innovent Biologics, Inc.), WBP3155 (CStone Pharmaceuticals Co., Ltd.), AMP-224 (GlaxoSmithKline plc), BI 754091 (Boehringer Ingelheim GmbH), BMS-936559 (Bristol-Myers Squibb Company), CA-170 (Aurigene Discovery Technologies), FAZ053 (Novartis AG), spartalizumab (PDR001, Novartis AG), LY3300054 (Eli Lilly & Company), MED10680 (AstraZeneca PLC), PDR001 (Novartis AG), cemiplimab (LIBTAYO®, REGN2810, Regeneron Pharmaceuticals, Inc.), camrelizumab (SHR-1210, Incyte Corporation), TSR-042 (Tesaro, Inc.), AGEN2034 (Agenus Inc.), CX-072 (CytomX Therapeutics, Inc.), JNJ-63723283 (Johnson & Johnson), MGD013 (MacroGenics, Inc.), AN-2005 (Adlai Nortye), ANA011 (AnaptysBio, Inc.), ANB011 (AnaptysBio, Inc.), AUNP-12 (Pierre Fabre Medicament S.A.), BBI-801 (Sumitomo Dainippon Pharma Co., Ltd.), BION-004 (Aduro Biotech), CA-327 (Aurigene Discovery Technologies), CK-301 (Fortress Biotech, Inc.), ENUM 244C8 (Enumeral Biomedical Holdings, Inc.), FPT155 (Five Prime Therapeutics, Inc.), FS118 (F-star Alpha Ltd.), hAb21 (Stainwei Biotech, Inc.), J43 (Transgene S.A.), JTX-4014 (Jounce Therapeutics, Inc.), KD033 (Kadmon Holdings, Inc.), KY-1003 (Kymab Ltd.), MCLA-134 (Merus B.V.), MCLA-145 (Merus B.V.), PRS-332 (Pieris AG), SHR-1316 (Atridia Pty Ltd.), STI-A1010 (Sorrento Therapeutics, Inc.), STI-A1014 (Sorrento Therapeutics, Inc.), STI-A1110 (Les Laboratoires Servier), and XmAb20717 (Xencor, Inc.). Additional anti-PD-1 and anti-PD-L1 antibodies ca nbe found in, e.g., Sunshine and Taube (2015) Curr. Opin. Pharmacol. 23:32; and Heery et al. (2017) The Lancet Oncology 18:587; Iwai et al. (2017) J. Biomed. Sci. 24:26; Hu-Lieskovan et al. (2017) Annals of Oncology 28: issue Suppl. 5, mdx376.048; WO2016/092419 and U.S. Patent Publication No. 2016/0159905.

In some cases, a suitable antibody is a bispecific antibody, e.g., a bispecific monoclonal antibody. Catumaxomab, blinatumomab, solitomab, pasotuxizumab, and flotetuzumab are non-limiting examples of bispecific antibodies suitable for use in cancer therapy. See, e.g., Chames and Baty (2009) MAbs 1:539; and Sedykh et al. (2018) Drug Des. Devel. Ther. 12:195.

Biological response modifiers suitable for use in connection with the methods of the present disclosure include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) interferon-α; (7) interferon-γ; (8) colony-stimulating factors; (9) inhibitors of angiogenesis; and (10) antagonists of tumor necrosis factor.

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™ TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Cell therapy includes chimeric antigen receptor (CAR) T cell therapy (CAR-T therapy); natural killer (NK) cell therapy; dendritic cell (DC) therapy (e.g., DC-based vaccine); T cell receptor (TCR) engineered T cell-based therapy; and the like.

Immune checkpoint inhibitors are known in the art and include antibodies specific for immune checkpoint polypeptide. For example, an immune checkpoint inhibitors include, e.g., an antibody specific for an immune checkpoint polypeptide selected from CD27, CD28, CD40, CD122, CD96, CD73, CD47, OX40, GITR, CSF1R, JAK, PI3K delta, PI3K gamma, TAM, arginase, CD137, ICOS, A2AR, B7-H3, B7-H4, BTLA, CTLA-4, LAG3, TIM3, VISTA, CD96, TIGIT, CD122, PD-1, PD-L1 and PD-L2.

Antibodies, e.g., monoclonal antibodies, that are specific for immune checkpoints and that function as immune checkpoint inhibitors, are known in the art. See, e.g., Wurz et al. (2016) Ther. Adv. Med. Oncol. 8:4; and Naidoo et al. (2015) Ann. Oncol. 26:2375.

Suitable anti-immune checkpoint antibodies include, but are not limited to, nivolumab (Bristol-Myers Squibb), pembrolizumab (Merck), pidilizumab (Curetech), AMP-224 (GlaxoSmithKline/Amplimmune), MPDL3280A (Roche), MDX-1105 (Medarex, Inc./Bristol Myer Squibb), MEDI-4736 (Medimmune/AstraZeneca), arelumab (Merck Serono), ipilimumab (YERVOY, (Bristol-Myers Squibb), tremelimumab (Pfizer), pidilizumab (CureTech, Ltd.), IMP321 (Immutep S.A.), MGA271 (Macrogenics), BMS-986016 (Bristol-Meyers Squibb), lirilumab (Bristol-Myers Squibb), urelumab (Bristol-Meyers Squibb), PF-05082566 (Pfizer), IPH2101 (Innate Pharma/Bristol-Myers Squibb), MEDI-6469 (Medlmmune/AZ), CP-870,893 (Genentech), Mogamulizumab (Kyowa Hakko Kirin), Varlilumab (CellDex Therapeutics), Galiximab (Biogen Idec), AMP-514 (Amplimmune/AZ), AUNP 12 (Aurigene and Pierre Fabre), Indoximod (NewLink Genetics), NLG-919 (NewLink Genetics), INCB024360 (Incyte); KN035; and combinations thereof.

Cancers

Cancer cells that may be treated by methods and compositions of the present disclosure include cells from the bladder, blood, bone, bone marrow, brain, breast, colon, esophagus, gastrointestine, gum, head, kidney, liver, lung, nasopharynx, neck, ovary, prostate, skin, stomach, spinal cord, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; pancreatic cancer; rectal cancer; and hairy cell leukemia.

Tumors that can be treated using a method of the present disclosure include, e.g., a brain cancer tumor, a head and neck cancer tumor, an esophageal cancer tumor, a skin cancer tumor, a lung cancer tumor, a thymic cancer tumor, a stomach cancer tumor, a colon cancer tumor, a liver cancer tumor, an ovarian cancer tumor, a uterine cancer tumor, a bladder cancer tumor, a testicular cancer tumor, a rectal cancer tumor, a breast cancer tumor, or a pancreatic cancer tumor.

In some cases, the tumor is a colorectal adenocarcinoma. In some cases, the tumor is non-small cell lung carcinoma. In some cases, the tumor is a triple-negative breast cancer. In some cases, the tumor is a solid tumor. In some cases, the tumor is a liquid tumor. In some cases, the tumor is recurrent. In some cases, the tumor is a primary tumor. In some cases, the tumor is metastatic.

A variety of subjects are suitable for treatment with a subject method of treating cancer. Suitable subjects include any individual, e.g., a human or non-human animal who has cancer, who has been diagnosed with cancer, who is at risk for developing cancer, who has had cancer and is at risk for recurrence of the cancer, who has been treated with an agent other than a an oncolytic vaccinia virus of the present disclosure for the cancer and failed to respond to such treatment, or who has been treated with an agent other than an oncolytic vaccinia virus of the present disclosure for the cancer but relapsed after initial response to such treatment.

Vaccinia Virus Immunogenic Compositions

The present disclosure provides a composition comprising an OVV comprising one or more of: a) a nucleotide sequence encoding a variant A33 polypeptide, wherein the variant A33 polypeptide provides for enhanced viral spreading and/or enhanced production of EEV, compared to a control vaccinia virus that does not comprise the nucleotide sequence encoding the variant A33 polypeptide (e.g., compared to a control vaccinia virus that comprises a nucleotide sequence encoding a wild-type A33 polypeptide, such as an A33 polypeptide having an amino acid sequence depicted in FIG. 1C); b) a nucleotide sequence encoding a variant A34 polypeptide, wherein the variant A34 polypeptide provides for enhanced viral spreading and/or enhanced production of EEV, compared to a control vaccinia virus that does not comprise the nucleotide sequence encoding the variant A34 polypeptide (e.g., compared to a control vaccinia virus that comprises a nucleotide sequence encoding a wild-type A34 polypeptide, such as an A34 polypeptide having an amino acid sequence depicted in FIG. 2C); and c) a nucleotide sequence encoding a variant B5 polypeptide, wherein the variant B5 polypeptide provides for enhanced viral spreading and/or enhanced production of EEV, compared to a control vaccinia virus that does not comprise the nucleotide sequence encoding the variant B5 polypeptide (e.g., compared to a control vaccinia virus that comprises a nucleotide sequence encoding a wild-type B5 polypeptide, such as a B5 polypeptide having an amino acid sequence depicted in FIG. 4D).

To induce or enhance an immune response in an individual to a cancer antigen, an OVV of the present disclosure is administered to an individual in need thereof. Subjects suitable for treatment include those described above. In some cases, the replication-competent, recombinant oncolytic vaccinia virus is administered to an individual in need thereof in a low dose, e.g., from about 10² plaque-forming units (pfu) to about 10⁴ pfu, from about 10⁴ pfu to about 10⁵ pfu, or from about 10⁵ pfu to about 10⁶ pfu per dose. In some cases, the replication-competent, recombinant oncolytic vaccinia virus is administered to an individual in need thereof in a dose of from about 10⁶ pfu to about 10¹² pfu, e.g., in a dose of from about 10⁶ pfu to about 10⁷ pfu, from about 10⁷ pfu to about 10⁸ pfu, from about 10⁸ pfu to about 10⁹ pfu, from about 10⁹ pfu to about 10¹⁰ pfu, from about 10¹⁰ pfu to about 10¹¹ pfu, or from about 10¹¹ pfu to about 10¹² pfu.

An OVV of the present disclosure can be administered to an individual in need thereof in a pharmaceutical composition, e.g., the pharmaceutical composition can comprise: a) an OVV of the present disclosure; and b) a pharmaceutically acceptable excipient. Thus, the present disclosure provides a pharmaceutical composition comprising: a) an OVV of the present disclosure; and b) a pharmaceutically acceptable excipient. Suitable pharmaceutically acceptable excipients are as described above. In some cases, the pharmaceutical composition comprises an adjuvant. Suitable adjuvants include, but are not limited to, alum, aluminum phosphate, aluminum hydroxide, MF59 (4.3% w/v squalene, 0.5% w/v Tween 80™ 0.5% w/v Span 85), CpG-containing nucleic acid (where the cytosine is unmethylated), monophosphoryl lipid A (MPL), 3-Q-desacyl-4′-monophosphoryl lipid A (3DMPL), and the like.

An OVV of the present disclosure can be administered to an individual in need thereof via any suitable route of administration, e.g., a route of administration as described above. For example, a recombinant vaccinia virus of the present disclosure can be administered to an individual in need thereof via an intramuscular, an intravenous, a subcutaneous route of administration.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-72 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. An OVV comprising one or more of:

a) a nucleotide sequence encoding a variant A33 polypeptide, wherein the variant A33 polypeptide provides for enhanced viral spreading and/or enhanced production of extracellular enveloped virion (EEV), compared to a control vaccinia virus that does not comprise the nucleotide sequence encoding the variant A33 polypeptide;

b) a nucleotide sequence encoding a variant A34 polypeptide, wherein the variant A34 polypeptide provides for enhanced viral spreading and/or enhanced production of EEV, compared to a control vaccinia virus that does not comprise the nucleotide sequence encoding the variant A34 polypeptide; and

c) a nucleotide sequence encoding a variant B5 polypeptide, wherein the variant B5 polypeptide provides for enhanced viral spreading and/or enhanced production of EEV, compared to a control vaccinia virus that does not comprise the nucleotide sequence encoding the variant B5 polypeptide.

Aspect 2. The replication-competent, recombinant oncolytic vaccinia virus of aspect 1, comprising:

a) a nucleotide sequence encoding a variant A33 polypeptide, wherein the variant A33 polypeptide provides for enhanced viral spreading and/or enhanced production of EEV, compared to a control vaccinia virus that does not comprise the nucleotide sequence encoding the variant A33 polypeptide; and

b) a nucleotide sequence encoding a variant A34 polypeptide, wherein the variant A34 polypeptide provides for enhanced viral spreading and/or enhanced production of EEV, compared to a control vaccinia virus that does not comprise the nucleotide sequence encoding the variant A34 polypeptide.

Aspect 3. The replication-competent, recombinant oncolytic vaccinia virus of aspect 1, comprising:

a) a nucleotide sequence encoding a variant A33 polypeptide, wherein the variant A33 polypeptide provides for enhanced viral spreading and/or enhanced production of EEV, compared to a control vaccinia virus that does not comprise the nucleotide sequence encoding the variant A33 polypeptide;

b) a nucleotide sequence encoding a variant A34 polypeptide, wherein the variant A34 polypeptide provides for enhanced viral spreading and/or enhanced production of EEV, compared to a control vaccinia virus that does not comprise the nucleotide sequence encoding the variant A34 polypeptide; and

c) a nucleotide sequence encoding a variant B5 polypeptide, wherein the variant B5 polypeptide provides for enhanced viral spreading and/or enhanced production of EEV, compared to a control vaccinia virus that does not comprise the nucleotide sequence encoding the variant B5 polypeptide.

Aspect 4. The replication-competent, recombinant oncolytic vaccinia virus of any one of aspects 1-3, wherein the variant A33 polypeptide comprises a substitution of 1, 2, or 3 of M63, A88, and E129.

Aspect 5. The replication-competent, recombinant oncolytic vaccinia virus of aspect 4, wherein the substitution is one or more of an M63R substitution, an A88D substitution, and an E129M substitution.

Aspect 6. The replication-competent, recombinant oncolytic vaccinia virus of aspect 5, wherein the variant A33 polypeptide comprises an A88D substitution and an E129M substitution.

Aspect 7. The replication-competent, recombinant oncolytic vaccinia virus of any one of aspects 4-6, wherein the vaccinia virus comprises a nucleotide sequence encoding a variant A34 polypeptide, wherein the variant A34 polypeptide comprises a K151E substitution.

Aspect 8. The replication-competent, recombinant oncolytic vaccinia virus of any one of aspects 1-3, wherein the variant A34 polypeptide comprises a substitution of 1, 2, 3, 4, or 5 of M66, F94, R84, R91, and T127.

Aspect 9. The replication-competent, recombinant oncolytic vaccinia virus of aspect 8, wherein the substitution is one or more of an M66T substitution, an F94H substitution, an R84G substitution, an R91A substitution, an R91S substitution, and a T127E substitution.

Aspect 10. The replication-competent, recombinant oncolytic vaccinia virus of aspect 9, wherein the variant A34 polypeptide comprises a K151E substitution.

Aspect 11. The replication-competent, recombinant oncolytic vaccinia virus of aspect 9, wherein the variant A34 polypeptide comprises an F94H substitution.

Aspect 12. The replication-competent, recombinant oncolytic vaccinia virus of aspect 10, wherein the variant A34 polypeptide comprises an F94H substitution and a K151E substitution.

Aspect 13. The replication-competent, recombinant oncolytic vaccinia virus of aspect 2,

wherein the variant A33 polypeptide comprises a substitution of one or more of M63, A88, and E129; and

wherein the variant A34 polypeptide comprises a substitution of one or more of M66, F94, R84, R91, and T127.

Aspect 14. The replication-competent, recombinant oncolytic vaccinia virus of aspect 13, wherein the variant A33 polypeptide comprises one or more of an M63R substitution, an A88D substitution, and an E129M substitution; and wherein the variant A34 polypeptide comprises one or more of an M66T substitution, an F94H substitution, an R84G substitution, an R91S substitution, an R91A substitution and a T127E substitution.

Aspect 15. The replication-competent, recombinant oncolytic vaccinia virus of aspect 14, wherein the variant A34 polypeptide comprises a K151E substitution.

Aspect 16. The replication-competent, recombinant oncolytic vaccinia virus of aspect 14, wherein the variant A33 polypeptide comprises an A88D substitution; and wherein the variant A34 polypeptide comprises an F94H substitution.

Aspect 17. The replication-competent, recombinant oncolytic vaccinia virus of aspect 14, wherein the variant A33 polypeptide comprises an E129M substitution and wherein the variant A34 polypeptide comprises an F94H substitution.

Aspect 18. The replication-competent, recombinant oncolytic vaccinia virus of aspect 14, wherein the variant A33 polypeptide comprises an A88D substitution and an E129M substitution, and wherein the variant A34 polypeptide comprises an F94H substitution.

Aspect 19. The replication-competent, recombinant oncolytic vaccinia virus of aspect 15, wherein the variant A33 polypeptide comprises an A88D substitution and an E129M substitution, and wherein the variant A34 polypeptide comprises a K151E substitution.

Aspect 20. The replication-competent, recombinant oncolytic vaccinia virus of aspect 15, wherein the variant A33 polypeptide comprises an A88D substitution, and wherein the variant A34 polypeptide comprises an F94H substitution and a K151E substitution.

Aspect 21. The replication-competent, recombinant oncolytic vaccinia virus of aspect 15, wherein the variant A33 polypeptide comprises an E129M substitution, and wherein the variant A34 polypeptide comprises an F94H substitution and a K151E substitution.

Aspect 22. The replication-competent, recombinant oncolytic vaccinia virus of aspect 15, wherein the variant A33 polypeptide comprises an A88D substitution and an E129M substitution, and wherein the variant A34 polypeptide comprises an F94H substitution and a K151E substitution.

Aspect 23. The replication-competent, recombinant oncolytic vaccinia virus of aspect 1 or aspect 3, wherein the variant B5 polypeptide comprises 1, 2, 3, 4, or more amino acid substitutions at positions of N39, L90, N94, S199, K229, V233, 1236, V238, T240, N241, E243, V247, D248, G250, D263, E268, E270, D272, S273, D275, and A276.

Aspect 24. The replication-competent, recombinant oncolytic vaccinia virus of aspect 23, wherein the variant B5 polypeptide comprises an S197F or an S197V substitution.

Aspect 25. The replication-competent, recombinant oncolytic vaccinia virus of aspect 23, wherein the variant B5 polypeptide comprises an S199M substitution.

Aspect 26. The replication-competent, recombinant oncolytic vaccinia virus of aspect 23, wherein the variant B5 polypeptide comprises an S273L substitution or an S273I substitution.

Aspect 27. The replication-competent, recombinant oncolytic vaccinia virus of aspect 23, wherein the variant B5 polypeptide comprises an N39G substitution.

Aspect 28. The replication-competent, recombinant oncolytic vaccinia virus of aspect 23, wherein the variant B5 polypeptide comprises an N94T substitution.

Aspect 29. The replication-competent, recombinant oncolytic vaccinia virus of aspect 23, wherein the variant B5 polypeptide comprises an L90R substitution and an S273V substitution.

Aspect 30. The replication-competent, recombinant oncolytic vaccinia virus of aspect 23, wherein the variant B5 polypeptide comprises an N39G substitution and an S273I substitution.

Aspect 31. The replication-competent, recombinant oncolytic vaccinia virus of aspect 23, wherein the variant B5 polypeptide comprises a K229C substitution and an S273L substitution.

Aspect 32. The replication-competent, recombinant oncolytic vaccinia virus of aspect 23, wherein the variant B5 polypeptide comprises an D263A substitution, an E270S substitution, an E272G substitution, and an E275F substitution.

Aspect 33. The replication-competent, recombinant oncolytic vaccinia virus of aspect 23, wherein the variant B5 polypeptide comprises an I236P substitution, a V238R substitution, a T240R substitution, and an E243G substitution.

Aspect 34. The replication-competent, recombinant oncolytic vaccinia virus of aspect 23, wherein the variant B5 polypeptide comprises a V233D substitution, an I236L substitution, a V238W substitution, a T240Y substitution, and an E243R substitution.

Aspect 35. The replication-competent, recombinant oncolytic vaccinia virus of aspect 23, wherein the variant B5 polypeptide comprises an D263V substitution, an E268T substitution, an E270G substitution, an E272P substitution, and an E275S substitution.

Aspect 36. The replication-competent, recombinant oncolytic vaccinia virus of aspect 23, wherein the variant B5 polypeptide comprises an N241T substitution, an E243V substitution, a V247S substitution, a G250R substitution, and an A276F substitution.

Aspect 37. The replication-competent, recombinant oncolytic vaccinia virus of aspect 23, wherein the variant B5 polypeptide comprises an N241G substitution, an E243S substitution, a V247W substitution, a D248Y substitution, a G250A substitution, and an A276F substitution.

Aspect 38. The replication-competent, recombinant oncolytic vaccinia virus of any one of aspects 1-37, comprising a nucleotide sequence encoding a variant A34 polypeptide, wherein the variant A34 polypeptide comprises a K151E substitution Aspect 39. The replication-competent, recombinant oncolytic vaccinia virus of any one of aspects 1-38, comprising a nucleotide sequence encoding a variant A56 polypeptide.

Aspect 40. The replication-competent, recombinant oncolytic vaccinia virus of aspect 39, wherein the variant A56 polypeptide comprises a substitution of amino acid 269.

Aspect 41. The replication-competent, recombinant oncolytic vaccinia virus of aspect 40, wherein the substitution of amino acid 269 is an I269F substitution. Aspect 42. The replication-competent, recombinant oncolytic vaccinia virus of any one of aspects 1-41, wherein the vaccinia virus comprises a heterologous nucleic acid comprising a nucleotide sequence encoding an immunomodulatory polypeptide.

Aspect 43. The replication-competent, recombinant oncolytic vaccinia virus of any one of aspects 1-41, wherein the vaccinia virus comprises a modification that results in lack of J2R expression and/or function.

Aspect 44. The replication-competent, recombinant oncolytic vaccinia virus of any one of aspects 1-41, wherein the vaccinia virus comprises both: i) a heterologous nucleic acid comprising a nucleotide sequence encoding an immunomodulatory polypeptide; and ii) a modification that results in lack of J2R expression and/or function.

Aspect 45. A composition comprising:

a) the vaccinia virus of any one of aspects 1-44; and

b) a pharmaceutically acceptable excipient.

Aspect 46. A method of inducing oncolysis in an individual having a tumor, the method comprising administering to the individual an effective amount of the vaccinia virus of any one of aspects 1-44, or the composition of aspect 45.

Aspect 47. The method of aspect 46, wherein said administering comprises administering a single dose of the virus or the composition.

Aspect 48. The method of aspect 47, wherein the single dose comprises at least 10⁶ plaque forming units (pfu) of the vaccinia virus.

Aspect 49. The method of aspect 47, wherein the single dose comprises from 109 to 10¹² pfu of the vaccinia virus.

Aspect 50. The method of aspect 46, wherein said administering comprises administering multiple doses of the vaccinia virus or the composition.

Aspect 51. The method of aspect 50, wherein the vaccinia virus or the composition is administered every other day.

Aspect 52. The method of aspect 50, wherein the vaccinia virus or the composition is administered once per week.

Aspect 53. The method of aspect 50, wherein the vaccinia virus or the composition is administered every other week.

Aspect 54. The method of any one of aspects 46-53, wherein the tumor is a brain cancer tumor, a head and neck cancer tumor, an esophageal cancer tumor, a skin cancer tumor, a lung cancer tumor, a thymic cancer tumor, a stomach cancer tumor, a colon cancer tumor, a liver cancer tumor, an ovarian cancer tumor, a uterine cancer tumor, a bladder cancer tumor, a testicular cancer tumor, a rectal cancer tumor, a breast cancer tumor, or a pancreatic cancer tumor.

Aspect 55. The method of any one of aspects 46-53, wherein the tumor is colorectal cancer.

Aspect 56. The method of any one of aspects 46-53, wherein the tumor is non-small cell lung cancer.

Aspect 57. The method of any one of aspects 46-53, wherein the tumor is breast cancer.

Aspect 58. The method of aspect 5576, wherein the tumor is a triple-negative breast cancer.

Aspect 59. The method of any one of aspects 46-58, wherein the tumor is a solid tumor.

Aspect 60. The method of any one of aspects 46-58, wherein the tumor is a liquid tumor.

Aspect 61. The method of any one of aspects 46-60, wherein the tumor is recurrent.

Aspect 62. The method of any one of aspects 46-60, wherein the tumor is a primary tumor.

Aspect 63. The method of any one of aspects 46-62, wherein the tumor is metastatic.

Aspect 64. The method of any one of aspects 46-63, further comprising administering to the individual a second cancer therapy.

Aspect 65. The method of aspect 64, wherein the second cancer therapy is selected from chemotherapy, biological therapy, radiotherapy, immunotherapy, hormone therapy, anti-vascular therapy, cryotherapy, toxin therapy, oncolytic virus therapy, a cell therapy, gene therapy, and surgery.

Aspect 66. The method of aspect 64, wherein the second cancer therapy is an immune checkpoint inhibitor.

Aspect 67. The method of aspect 66, wherein the immune checkpoint inhibitor is an antibody specific for an immune checkpoint inhibitor selected from CD27, CD28, CD40, CD122, CD96, CD73, CD47, OX40, GITR, CSF1R, JAK, PI3K delta, PI3K gamma, TAM, arginase, CD137, ICOS, A2AR, B7-H3, B7-H4, BTLA, CTLA-4, LAG3, TIM3, VISTA, CD96, TIGIT, CD122, PD-1, PD-L1 and PD-L2.

Aspect 68. The method of any one of aspects 46-67, wherein the individual is immunocompromised.

Aspect 69. The method of any one of aspects 46-68, wherein said administering of the vaccinia virus or the composition is intra-arterial, intraperitoneal, intrabladder, or intrathecal.

Aspect 70. The method of any one of aspects 46-68, wherein said administering of the vaccinia virus or the composition is intratumoral.

Aspect 71. The method of any one of aspects 46-68, wherein said administering of the vaccinia virus or the composition is peritumoral.

Aspect 72. The method of any one of aspects 46-68, wherein said administering of the vaccinia virus or the composition is intravenous.

Aspect 73 The vaccinia virus of any one of aspects 1-44, or the composition of aspect 45 for use in a method of inducing oncolysis in an individual having a tumor.

Aspect 74. Use of the vaccinia virus of any one of aspects 1-44, or the composition of aspect 45 in the manufacture of a medicament for use in a method of inducing oncolysis in an individual having a tumor.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

A. Generation of Vaccinia Virus Library Backbones

Backbone viruses were generated to facilitate cloning, reactivation, and construction of multiple libraries into loci of interest within the vaccinia virus genome. Backbone viruses contained the selectable markers herpes simplex virus-thymidine kinase (HSV-TK) and mCherry fusion protein (codon optimized for vaccinia virus expression) under the control of the synthetic early late promoter (pSEL). Donor DNA containing the pSEL-HSV-TK/mCherry cassette flanked by homing endonuclease sites I-Scel and I-Ceul, and unique pairs of Aarl sites in upstream and downstream regions of homology to 1) the A33R/A34R loci, 2) the A56R locus, or 3) the B5R locus were generated via overlapping polymerase chain reaction (PCR) with specific primers for each region (IDT) and Phusion-HF (Thermo Fisher). PCR products were purified using Zymo DNA clean-and-concentrator-5 kit (ZymoResearch) and blunt-cloned into the DNA shuttle vector supplied by the Zero Blunt™ TOPO™ PCR Cloning Kit (Thermo Fisher). DNA was sequenced by Elim Biopharmaceuticals (Hayward, Calif.). Viral genomic DNA from Copenhagen vaccinia virus containing a J2R deletion and insertion of firefly luciferase-2A-eGFP driven by the pSEL promoter, was extracted and cut at one of the three loci of interest. The selectable markers replaced each loci of interest: 1) A33R/A34R loci, 2) A56R locus, and 3) the B5R locus of the vaccinia virus genome using viral reactivation. Briefly, VERO-B4 cells (DSMZ) were infected with Shope fibroma virus (SFV; ATCC) for 2 hours at room temperature, washed and allowed to recover at 37° C. and 5% CO₂ for 1 hour. During recovery, transfection mixture containing genomic DNA and donor DNA were mixed with Lipofectamine 2000 (Thermo Fisher) according to manufacturer instructions. The infected cells were transfected with the transfection mixture at 37° C. and 5% CO₂ for 2 days. Functional virus was recovered by serial dilution and plaque isolation from BSC-40 cells (Earl et al. (1998) Curr. Protocol. Mol. Biol. 43:16.17.1.). Virus plaques were DNA extracted using QuickExtract (Epicentre/Lucigen) and Sanger sequenced before and after 3 rounds of plaque purification by Elim Biopharmaceuticals. Sequence verified viruses were amplified in HeLa S3 cells (DSMZ), and purified as described below. DNA was extracted (Cotter et al, (2015) Curr. Protocol Mol. Biol. 39:14A.3.1.) and summited for whole genome sequencing (WGS) (Seqmatic Hayward, Calif.). WGS analysis was performed in-house using Geneious desktop software (Biomatters Ltd.)

B. Generation of Vaccinia Virus A33R, A34R, A56R, and B5R DNA Libraries

Libraries containing single amino acid substitutions were generated via PCR. Template DNA containing the Copenhagen vaccinia virus sequences for A33R and A34R (containing a K151E substitution), A56R, or B5R were synthesized (Genescript) for each locus with unique Aarl and Sfil cloning sites inserted upstream of the first gene's promoter and downstream of the last gene's terminator. NNK libraries spanning the length of the genes were designed for A33R, A34R, A56R, and B5R coding sequences. Degenerate NNK-containing primers (IDT) tiled across the coding regions of each protein were used to generate NNK variants for each locus by PCR.

Homology between forward and reverse primers permitted self-assembly of NNK variants for each codon using NEBuilder® HiFi DNA Assembly (NEB). Assembly reactions were transformed into Endura chemically competent E. coli (Lucigen) and grown on selective agar media (Luria Broth with 100 μg/mL carbenicillin or LB+Carb, Teknova). Colonies were counted and transformations were continued until ˜20× colony forming units (CFUs) per NNK was achieved for each codon in each locus. Colonies were recovered from the plates by flooding and scraping with liquid media LB+Carb (Teknova), aliquoted, and frozen in 30% glycerol. One aliquot for each codon was inoculated into a 96-well plate containing LB+50 μg/mL Carb, shaking overnight at 37° C. and 900 rpm; then miniprepped using the Nucleospin 96 Plasmid Transfection-grade Kit (Macherey-Nagel). Minipreps were diluted 1:100 in sterile water; then the donor DNA for each codon was amplified with specific primers (IDT) for A33R/A34R, A56R, and B5R respectively using Phusion-HF (Thermo Fisher). PCR amplicons for each randomized codon were quantified using the AccuClear Ultra High Sensitivity dsDNA quantification kit (Biotium) on a Spectramax M5 plate reader (Molecular Devices) and sequenced by Elim Biopharmaceuticals (Hayward, Calif.). Sequencing results were analyzed in-house using Geneious (Biomatters Ltd.). Verified NNK PCR products were normalized by concentration, pooled and used as donor DNA to recombine into vaccinia virus. Libraries containing randomized regions of interest within the B5R genes were synthesized by Genewiz using a degenerate synthesis process at the defined regions of randomization. Library diversity estimates and allele frequencies at the DNA library stage were estimated for all the libraries.

C. Generation of Vaccinia Virus A33, A34, A56, and B5 Viral Libraries

The libraries were inserted into the vaccinia virus genome using the same strategy described for vaccinia backbone generation or viral reactivation. Briefly, VERO-B4 cells (DSMZ) were infected with Shope fibroma virus (SFV, ATCC) and transfected with digested viral genomic DNA and donor DNA. Vaccinia virus viral particles were recovered 24 hours later. Diversity immediately after virus reactivation was estimated by isolating plaques, extracting DNA with Quick Extract (Epicentre/Lucigen), and sequencing PCR amplicons as described above. Viruses from each library were amplified in HeLa S3 cells. Virus seed stocks were analyzed for diversity by amplifying each locus and submitting for amplicon next-generation sequencing (NGS, SeqMatic). Analysis of the diversity present in each locus-specific library was facilitated by the free command-line program VirVarSeq (at https://omictools(dot)com(forward slash)virvarseq-tool)) and post-processing using Microsoft Excel. Amplified viral libraries were purified by sucrose gradient and ultracentrifugation (Cotter et al. (2015) Curr. Protocol Mol. Biol. 39:14A.3.1), as described below. Purified viral libraries were stored at −80° C. and titered in duplicate by plaque assay, adding serial dilutions of purified virus to BSC-40 cells (ATCC) as described below (Earl et al. (1998) Curr. Protocol. Mol. Biol. 43:16.17.1.).

D. Directed Evolution

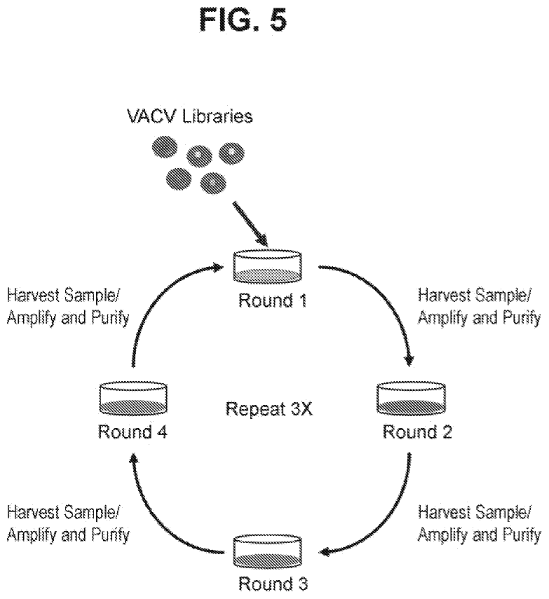

Several in vitro and in vivo directed evolution programs were undertaken to identify variants capable of increased EEV production and virus spreading in combination with transduction of various tumor types. The programs are summarized in Table 1. The general directed evolution process is summarized in FIG. 5.

TABLE 1

| Selection | Cell Line | Tumor Type | Serum |
|---|---|---|---|
| in vitro | primary cancer cells | colorectal; breast; | — |
| | and VEGF | non-small cell lung; | |
| | stimulated | normal VEGF | |
| | endothelial cells | stimulated | |
| | | endothelial | |
| in vitro | HCT 116 | colorectal | — |
| in vitro | Colo 205 | colorectal | — |
| in vitro | MDA-MB-231 | breast | — |
| in vitro | MDA-MB-231 | breast | vaccinated |
| | | | donors |
| in vivo | patient derived | colorectal | — |
| | xenograft | | |
| | in NSG mouse | | |
| in vivo | patient derived | colorectal | — |
| | xenograft | | |
| | in humanized mouse | | |

FIG. 5 provides a schematic example of a single stage of the directed evolution process used to identify EEV variants in vitro. Vaccinia virus containing libraries of viral variants in the appropriate region of interest, in this example A33/A34, B5 and A56 regions, were first engineered through large-scale viral reactivation experiments before being subjected to multiple rounds of selection in particular cell types. The directed evolution process can be adapted to either cancer patient-derived primary cells or immortalized cancer cell-lines. Viral variants can pass through rounds of selection in one or a variety of cell types. Briefly, the process begins with virus capable of replication and release in the supernatant in the first cell type chosen, followed by harvesting after a 24-hour incubation period, amplification and purification (Round 1). The infectious virus obtained from Round 1 is then used to infect either another cell type or the same cell type (Round 2). The execution of Rounds 1-4 is considered 1 Stage of the directed evolution process. Altogether, 3 total Stages were completed. Depending on the cell type being used (cancer patient-derived primary cells or immortalized cancer cell-lines) various multiplicities of infection (MOIs) should be considered.

For multiple rounds of the directed evolution process, sequencing of a representative fraction of the library was performed using a combination of amplicon next-generation sequencing (NGS), Sanger sequencing of individual plaques, and whole genome sequencing (WGS). The frequency, expressed as a percentage of the total population, of the most prevalent variants increased significantly over the course of the selections.

E. Plasmid Construction

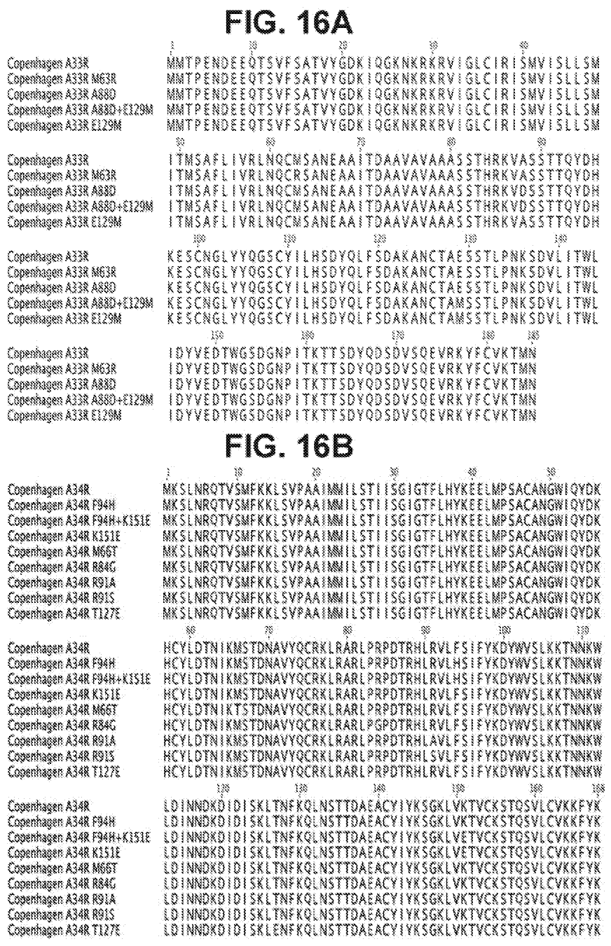

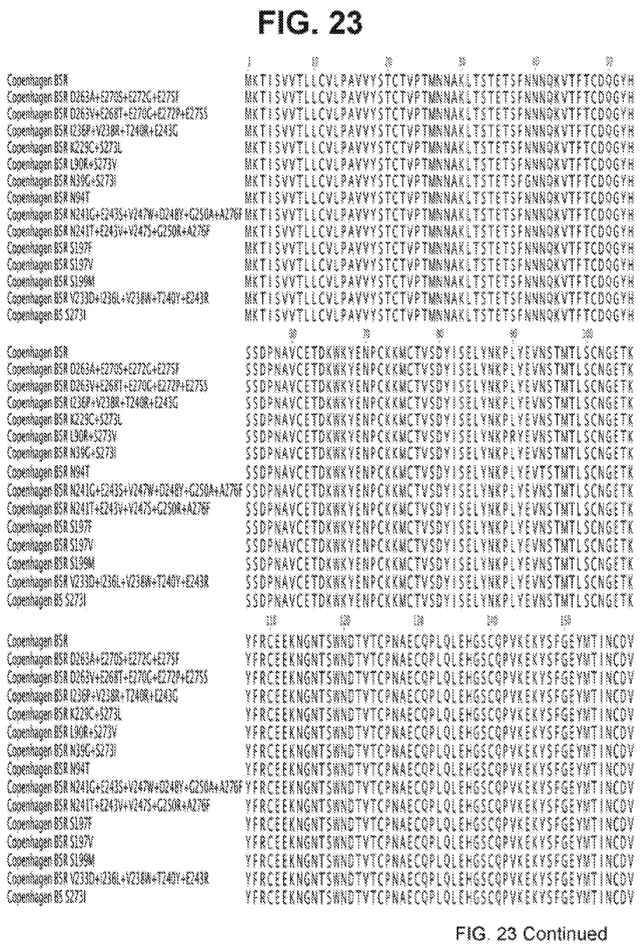

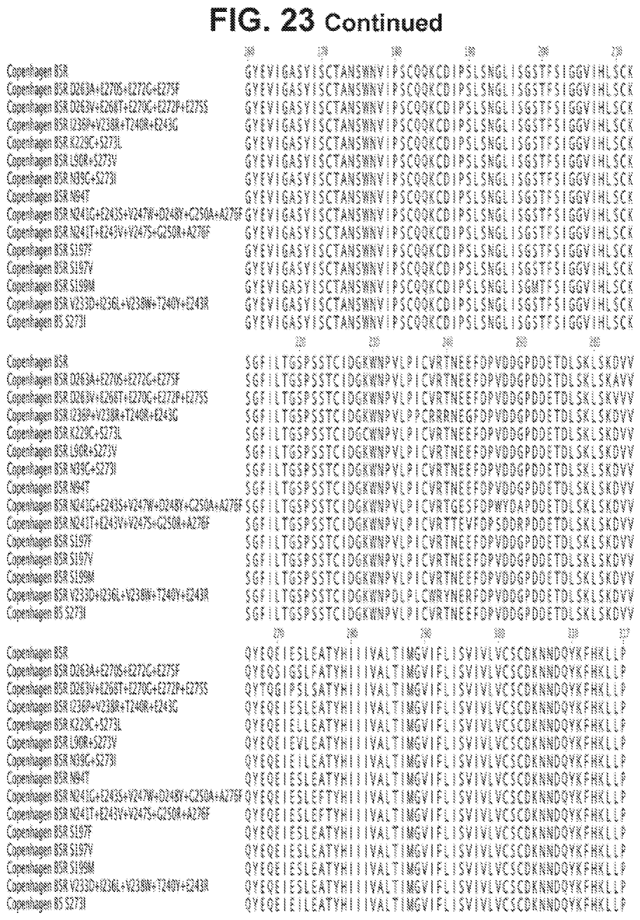

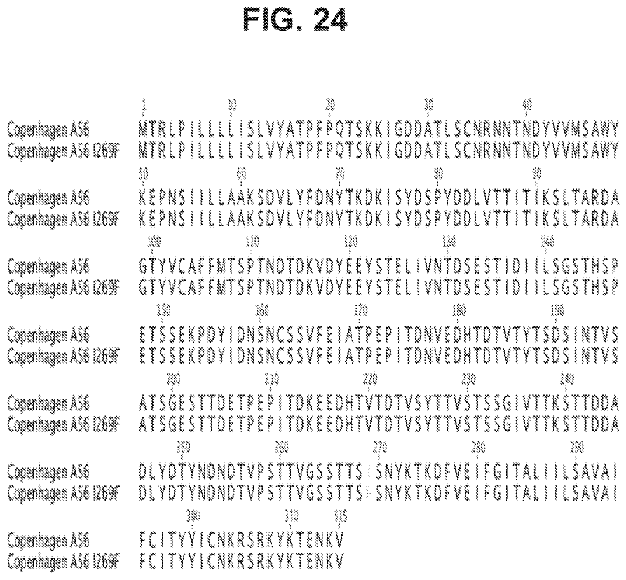

Plasmids containing variant A33R and/or A34R sequences were generated using gene synthesis techniques. A sequence encoding each of the substitution variants described below was submitted to GenScript for gene synthesis and inserted into the pUC57-mini vector. The amino acid sequences encoded by the variant A33 and A34 sequences are annotated in in the Brief Description of FIG. 16 as SEQ ID NO:57-60, 61-67, and 81. The amino acid sequences encoded by the variant B5 sequences are annotated in in the Brief Description of FIG. 23 as SEQ ID NO:68-79, 82, and 83. The amino acid sequence encoded by the variant A56 sequence is annotated in the Brief Description of FIG. 24 as SEQ ID NO:80.

F. Viruses and Cells

Vaccinia virus Copenhagen IGV-007 was constructed by homologous recombination insertion of a luciferase-2A-GFP cassette under the control of the pSEL promoter into the thymidine kinase viral gene J2R of the Copenhagen strain of vaccinia virus. The expression of the luciferase reporter gene was confirmed by luminescence using the Bright-Glo™ Luciferase Assay System (Promega) and a Spectramax M5 (Molecular Devices). GFP expression was confirmed via fluorescent microscopy.

Vaccinia virus Copenhagen strains containing the K151E substitution in A34 (IGV-006), the A88D substitution in A33 (IGV-060), the E129M substitution in A33 (IGV-061), the F94H substitution in A34 (IGV-062), the A88D and E129M substitutions in A33 (IGV-063), the A88D substitution in A33 and the F94H substitution in A34 (IGV-064), the E129M substitution in A33 and the F94H substitution in A34 (IGV-065), and the A88D and E129M substitutions in A33 and the F94H substitution in A34 (IGV-066) were constructed by reactivation and homologous recombination of the synthesized genes into the A33R/A34R gene region of IGV-007. Vaccinia virus Copenhagen strains containing the A88D substitution in A33 and the K151E substitution in A34 (IGV-067), the E129M substitution in A33 and the K151E substitution in A34 (IGV-068), the F94H and K151E substitutions in A34 (IGV-073), the A88D and E129M substitutions in A33 and the K151E substitution in A34 (IGV-069), the A88D substitution in A33 and the F94H and K151E substitutions in A34 (IGV-070), the E129M substitution in A33 and the F94H and K151E substitutions in A34 (IGV-071), and the A88D and E129M substitutions in A33 and the F94H and K151E substitutions in A34 (IGV-072) were constructed by reactivation and homologous recombination of the synthesized genes into the A33R/A34R gene region of IGV-006. Successful insertion of the variant A33R gene and/or A34R gene into IGV-007 or IGV-006 was verified by Sanger sequencing of individual isolated plaques. Viruses were amplified and purified as described below.

Vaccinia virus Western Reserve strains containing the K151E substitution in A34 (IGV-117), the F94H and K151E substitutions in A34 (IGV-118), the A88D substitution in A33 and the F94H and K151E substitutions in A34 (IGV-119), and the A88D and E129M substitutions in A33 and the F94H and K151E substitutions in A34 (IGV-120) were constructed by reactivation and homologous recombination of the synthesized genes into the A33R/A34R gene region of IGV-013. Vaccinia virus Western Reserve (WR; ATCC) IGV-013 was constructed by homologous recombination insertion of a luciferase-2A-GFP cassette under the control of the pSEL promoter with a tetracycline operator into the thymidine kinase viral gene J2R of the WR strain of vaccinia virus. The expression of the luciferase reporter gene was confirmed by luminescence using the Bright-Glo™ Luciferase Assay System (Promega) and a Spectramax M5 (Molecular Devices). GFP expression was confirmed via fluorescent microscopy.

HeLa, U-2 OS, and BSC-40 cells were obtained from ATCC. A549, HCT 116, Colo205, and MDA-MB-231 cells were obtained from the NCI DCTD Repository of Tumors and Tumor Cell Lines. HeLa S3 and VERO-B4 cells were obtained from DSMZ. Cancer patient-derived primary cells (Breast, Colorectal, and Lung) were obtained from Conversant Bio. Human Umbilical Vein Endothelial Cells (HUVEC) were obtained from Lonza. RK-13 cells were obtained from Sigma-Aldrich.

G. Virus Amplification and Purification

HeLa S3 cells (DSMZ) were infected by adding virus to the monolayer and incubating for 1 hour in media with 2.5% FBS. Following infection, fresh media was added and the infected cells monolayer was incubated for 48 hours to allow for virus replication and amplification. Following incubation, the cells were harvested and collected by centrifugation. Cells were lysed by mechanical disruption with a Dounce homogenizer (Wheaton). Virus purification was accomplished with a 24% to 40% sucrose gradient and ultracentrifugation. Purified virus was aliquoted, stored at −80° C. and titered in duplicate by plaque assay, adding serial dilutions of purified virus to BSC-40 cells or U-2 OS cells (ATCC) as previously described (Earl et al. (1998) Curr. Protocol. Mol. Biol. 43:16.17.1.).

H. Virus Titering by Plaque Assay

Virus titer was determined by ten-fold serial dilutions, with a final dilution of 10-9 of the stock concentrated, purified virus. The virus dilutions were used to infect BSC-40 cells or U-2 OS cells to determine the number of plaque forming units per mL (PFU/mL). 1 mL of each serial dilution was applied in duplicate to wells containing a confluent monolayer of cells in a standard 6-well microplate (BD Falcon). Cells were infected for an hour, washed with fresh media, and overlaid with a solution of fresh media containing 1.5% carboxymethylcellulose (Teknova). Following 48 or 72 hours of incubation, the media was removed, and the cells were fixed and stained with a 20% ethanol solution containing 0.1% crystal violet (Sigma). The stock titer was then determined by counting the number of plaques in each well, averaging between duplicate titers, and adjusting for the dilution factor.

I. Infectious Virus in Supernatant of Human Tumor Cells

Virus replication in the tumor cell lines A549 (NCI), Colo205 (NCI), MDA MB 231 (NCI), HT-29 (NCI), SW-620 (NCI), and HeLa (DSMZ) was determined by infecting a monolayer of cells with virus at an MOI of 1 or 3 for 1 hour in triplicate. Following infection, the viral inoculum was washed 3 times and replaced with fresh media. Virus produced in the supernatant and cells were harvested separately into media at 24 hours post-infection. The virus in the supernatant was titered immediately in duplicate using the plaque assay protocol described above. The virus contained in the cell pellet was frozen and stored at −80° C.

J. Comet Assay

Monolayers of BSC-40 cells seeded in 6-well plates were infected with 1 mL of 10-fold serial dilutions of virus for 1 hour. The infected cells were washed 3 times, replenished with fresh media and incubated for 48 or 72 hours at a 40° angle at 37° C. Cell monolayers were stained with a 20% ethanol solution containing 0.1% crystal violet (Sigma) for 2 hours to visualize comets.

K. Spreading Assay

For a two-stage infectivity assay, U-2 OS (osteosarcoma), RK-13 (rabbit kidney), VERO-B4 (African green monkey kidney), A549 (lung adenocarcinoma) or MDA-MB-231 (breast cancer) cells were infected with a range of MOIs for 1 hour. The infected cells were washed 3 times and incubated for 20-24 hours, at which time the supernatant was collected. The supernatant was clarified from cell debris by low speed centrifugation and used to infect a new plate of the corresponding cells seeded in 96-well white wall plate (Greiner). Fifteen or twenty four hours post-infection on the second plate, luciferase activity was assessed on a Spectramax M5 spectrophotometer using the Bright-Glo Luciferase Assay System (Promega).

L. EEVs Quantification

Virus samples containing enrichment of intracellular mature virus (IMV) particles or enrichment of extracellular enveloped virus (EEV) particles were separated and purified using a CsCl density gradient ultracentrifugation method. Virus was overlaid on a 25% to 30% CsCl gradient and centrifuged for 18 hours at 175,000 rcf. Bands containing the enriched fractions were extracted at defined locations within the density gradient, and CsCl was removed. Samples were incubated with a conjugated anti-B5 antibody prior to quantification. Virus sized particles (VSP) and VSP containing the B5 antigen (an indicator of EEV) were quantified using a Micro flow cytometer (Apogee).

### Example 2

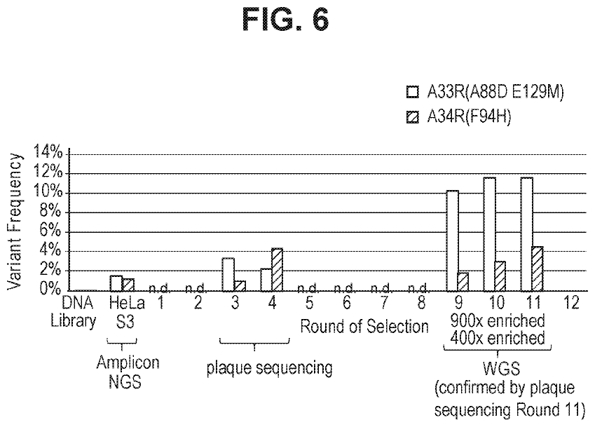

The first selection identified variants capable of enhanced spreading and EEV production following infection of different primary cancer cells (colon, breast and lung) and VEGF stimulated endothelial cells. Two variants were identified that demonstrated an increased frequency within the sequenced population (FIG. 6). The two variants, one containing A88D and E129M substitutions to the A33 sequence and one containing an F94H substitution to the A34 sequence showed substantial enrichment (900-fold and 400-fold, respectively) in Round 11 (FIG. 6).

FIG. 6 provides data on the frequency of specific vaccinia virus variants in various rounds of the directed evolution process to identify variants capable of enhanced spreading and EEV production following infection of different human primary cancer cells and VEGF stimulated endothelial cells. A representative fraction of the library was sequenced following initial HeLa S3 amplification, Round 3, Round 4, Round 9, Round 10, and Round 11 using a combination of amplicon next-generation sequencing (NGS), Sanger sequencing of individual plaques (plaque sequencing), and whole genome sequencing (WGS). The frequency of the two most prevalent variants, expressed as a percentage of the total population, increased significantly over the course of the selection. Enrichment for an A33 variant containing A88D and E129M substitutions and an A34 variant containing F94H and K151E substitutions demonstrates that these substitutions increase the ability of vaccinia virus to increase spreading and produce EEVs following infection of primary human cancer cells. n.d., not done.

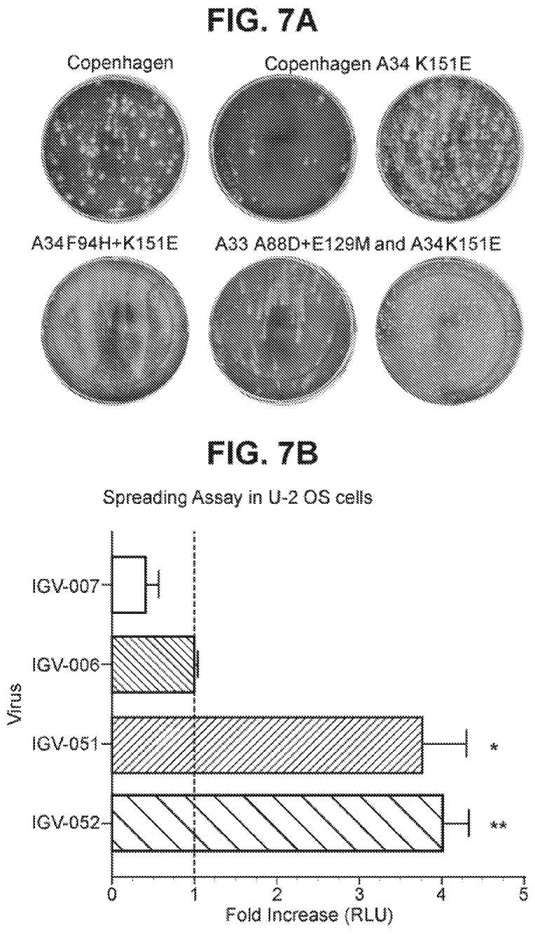

The impact of the substitution variants on viral spreading and EEV production was assessed in vitro using BSC-40 (African green monkey kidney) and U-2 OS (human osteosarcoma) cell lines. Copenhagen vaccinia viruses containing the wild-type A33 and A34 sequences (IGV-007), a K151E substitution in A34 (IGV-006; a known mutation to increase spreading and EEV production), the A88D and E129M substitutions in A33 and a K151E substitution in A34 (IGV-051), or the F94H and K151E substitutions in A34 (IGV-052) were generated and manufactured as described in Example 1. The comet assay was performed as described in Example 1. The presence of longer comet tails (FIG. 7A) for the variant vaccinia viruses compared to Copenhagen vaccinia virus containing the wild-type A33 and A34 sequences indicates enhanced virus spreading and therefore more EEVs being produced by the variant vaccinia viruses, specifically for IGV-051 and IGV-052. The spread of the variants was further assessed using the spreading assay described in Example 1. When compared to Copenhagen vaccinia viruses containing the wild-type A33 and A34 sequences (IGV-007) and a K151E substitution in A34 (IGV-006), the variant vaccinia viruses (IGV-051 and IGV-052) generated a higher quantity of relative light units (RLU), indicating enhanced viral spreading (FIG. 7B). These studies illustrate that incorporation of substitutions to A33 and A34, in particular at the A88 and E129 locations in A33 and the F94 location in A34 in combination with the K151E substitution in A34, leads to enhanced EEV production and therefore virus spreading.

FIG. 7A-7B provide data on virus spreading and EEV production of vaccinia virus variants containing A33 and A34 substitutions. A) Representative images of comets formed following infection of BSC-40 cells demonstrate that the vaccinia virus variants result in longer comet tails, an indication of better spreading and enhanced EEV production, compared to Copenhagen vaccinia virus containing the wild-type A33 and A34 sequences (top left) and Copenhagen vaccinia virus containing a K151E substitution in A34 (a known mutation to increase virus spreading and EEV production; top right). B) A two-stage infectivity assay to measure viral spreading, in which U-2 OS cells were first infected with different 3-fold dilutions of multiplicities of infection (MOI) of vaccinia virus, then supernatant was collected 22 hours post-infection, clarified and used to infect a new plate of U-2 OS cells. Luciferase expressed from the virus is measured at 15 hours post-infection, and increased levels of luciferase correlate with higher rates of infection and virus replication. Thus, a higher luciferase level is indicative of increased spreading. Data is provided as the fold increase compared to luciferase expression in IGV-006 (Copenhagen vaccinia virus containing a K151E substitution in A34). Both vaccinia virus variants show nearly seven-fold increases in viral spreading compared to a Copenhagen virus with no substitutions in A34 (IGV-007), which could lead to improved oncolytic activity in cancer by a better intratumoral spreading capability. IGV-007 is Copenhagen (control), IGV-006 is Copenhagen A34 K151E substitution, IGV-051 is Copenhagen A34 F94H and K151E substitutions, IGV-052 is A33 A88D and E129M and A34 K151E substitutions. Error bars indicate SD (n=3). Asterisks indicate statistical significance against Copenhagen vaccinia virus with K151E substitution in A34R gene (IGV-006) (*p<0.05; **p<0.001; Student's t-test).

### Example 3

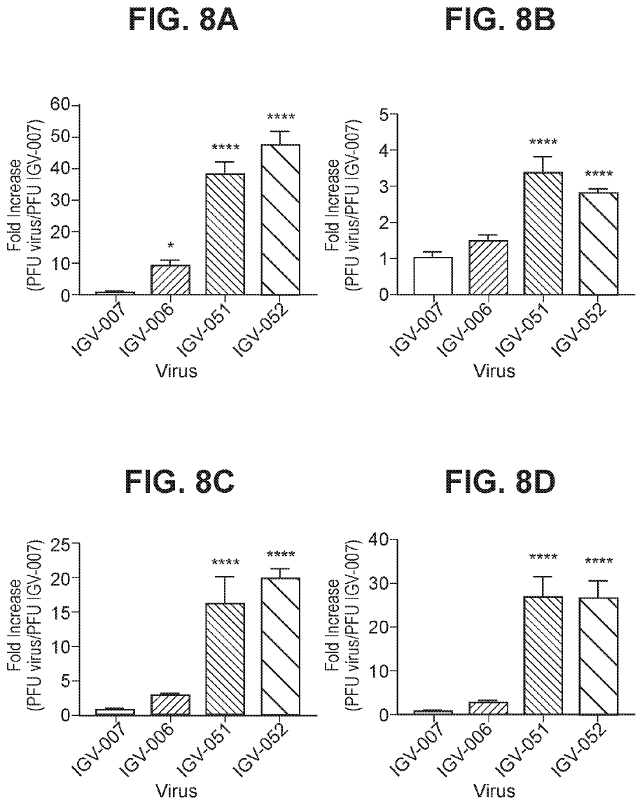

The impact of the substitution variants on infectious virus generated in the supernatant (EEV production) in different human cancer cell lines was assessed in vitro using A549 cells (lung adenocarcinoma), MDA-MB-231 cells (breast adenocarcinoma), Colo205 cells (colorectal adenocarcinoma), and HeLa cells (cervical cancer). Copenhagen vaccinia viruses containing the wild-type A33 and A34 sequences (IGV-007), a K151E substitution in A34 (IGV-006; a known mutation to increase virus spreading and EEV production), the A88D and E129M substitutions in A33 and a K151E substitution in A34 (IGV-051), or the F94H and K151E substitutions in A34 (IGV-052) were generated and manufactured as described in Example 1. All cell lines listed above were infected with a MOI of 1 and washed thoroughly 3 times after 1 hour of virus adsorption. At 24 hours post-infection, the supernatant was collected, and the number of infectious viruses produced in the supernatant (potentially EEVs) was determined via plaque assay, as described in Example 1. In all four cell lines, the substitution variants containing the A88D and E129M substitutions in A33 and a K151E substitution in A34 (IGV-051) or the F94H and K151E substitutions in A34 (IGV-052) produced a higher number of infectious viruses in the supernatant (potentially EEVs) (FIG. 8A-8D). This study demonstrates that incorporation of mutations to A33 and A34, in particular at the A88 and E129 locations in A33 and the F94 location in A34 in combination with the K151E substitution in A34, leads to enhanced infectious EEV production in different cancer cell lines.

FIG. 8A-8D provide data on vaccinia virus production of infectious virus released to the supernatant early on the infection cycle (potentially EEVs) in representative human cancer cell lines. A) A549 cells (lung adenocarcinoma), B) MDA-MB-231 cells (breast adenocarcinoma), C) Colo205 cells (colorectal adenocarcinoma), and D) HeLa cells (cervical cancer) were infected with vaccinia virus with a MOI of 1. Twenty-four hours post-infection, the number of infectious viruses released to the supernatant (potentially EEV particles) were determined by plaque assay. The fold increase of infectious viruses released in the supernatant in each cancer cell line was calculated for each virus variant compared to a Copenhagen virus containing the wild-type A33 and A34 sequences (IGV-007). There is a significant increase of infectious viruses in the supernatant for both vaccinia virus variants compared to Copenhagen containing the wild-type A33 and A34 sequences and Copenhagen vaccinia virus containing a K151E substitution in A34 (a known mutation to increase virus spreading and EEV production) in all cancer cell lines tested. IGV-007 is Copenhagen (control), IGV-006 is Copenhagen A34 K151E substitution, IGV-051 is Copenhagen A34 F94H and K151E substitutions, IGV-052 is A33 A88D and E129M and A34 K151E substitutions. Error bars indicate SD (n=3). Asterisks indicate statistical significance against Copenhagen vaccinia virus containing the wild-type A33 and A34 sequences (IGV-007) (*p<0.5, ****<0.0001; one-way ANOVA and Dunnett's multiple comparison test).

### Example 4

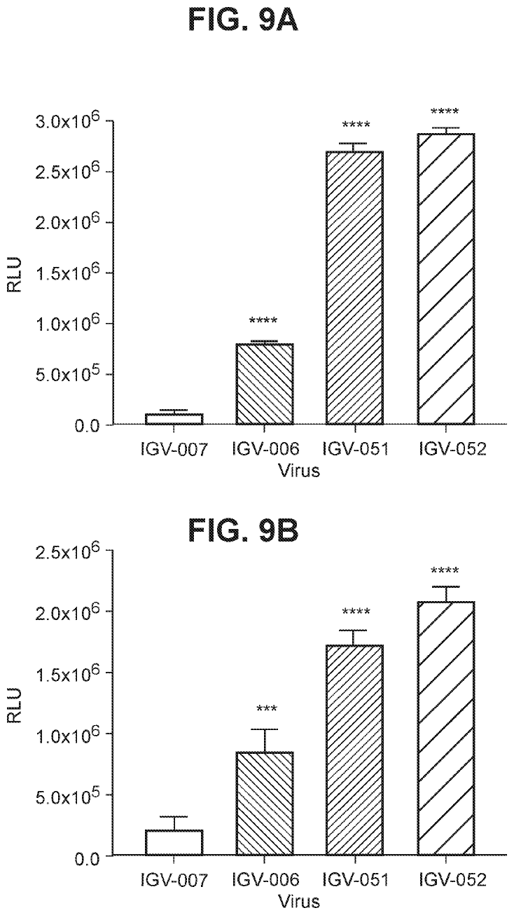

The impact of the substitution variants on vaccinia virus spreading in different human cancer cell lines was assessed in vitro using A549 cells (lung adenocarcinoma), and MDA-MB-231 cells (breast adenocarcinoma). Copenhagen vaccinia viruses containing the wild-type A33 and A34 sequences (IGV-007), a K151E substitution in A34 (IGV-006; a known mutation to increase virus spreading and EEV production), the A88D and E129M substitutions in A33 and a K151E substitution in A34 (IGV-051), or the F94H and K151E substitutions in A34 (IGV-052) were generated and manufactured as described in Example 1. A549 cells (lung adenocarcinoma), and MDA-MB-231 cells (breast adenocarcinoma) were infected with serial dilutions of MOIs of all the viruses tested, and the cells were washed 3 times after 1 hour. The supernatant was collected at 22 hours post-infection, clarified, and used to infect a new plate of cells. After 15 hours post-infection, the luciferase activity in cells on the second plate was assessed by quantifying luminescence using Bright-Glo Luciferase assay system (Promega). In both cell lines, the substitution variants containing the A88D and E129M substitutions in A33 and a K151E substitution in A34 (IGV-051) or the F94H and K151E substitutions in A34 (IGV-052) led to higher luciferase detection (FIG. 9A-9B), which is indicative of superior infection and spreading. This study further illustrates that incorporation of substitutions to A33 and A34, in particular at the A88 and E129 locations in A33 and the F94 location in A34 in combination with the K151E substitution in A34, leads to enhanced viral spreading in different human cancer cells lines.

FIG. 9A-9B provide data on vaccinia virus spreading in representative human cancer cell lines. A) A549 cells (lung adenocarcinoma), and B) MDA-MB-231 cells (breast cancer) were infected with vaccinia virus at MOI of 1.25 for 1 hour. Twenty-two hours post-infection, supernatant was collected, clarified, and used to infect a new plate of cells. Luciferase activity (measured as RLU) was determined 15 hours post-secondary infection. Higher luciferase activity was observed in the variant vaccinia viruses, demonstrating that they are capable of stronger spread in different human cancer cells. IGV-007 is Copenhagen (control), IGV-006 is Copenhagen A34 K151E substitution, IGV-051 is Copenhagen A34 F94H and K151E substitutions, IGV-052 is A33 A88D and E129M and A34 K151E substitutions. Error bars indicate SD (n=3). Asterisks indicate statistical significance against Copenhagen vaccinia virus containing the wild-type A33 and A34 sequences (IGV-007) (***p<0.001, ****<0.0001; one-way ANOVA and Dunnett's multiple comparison test).

### Example 5

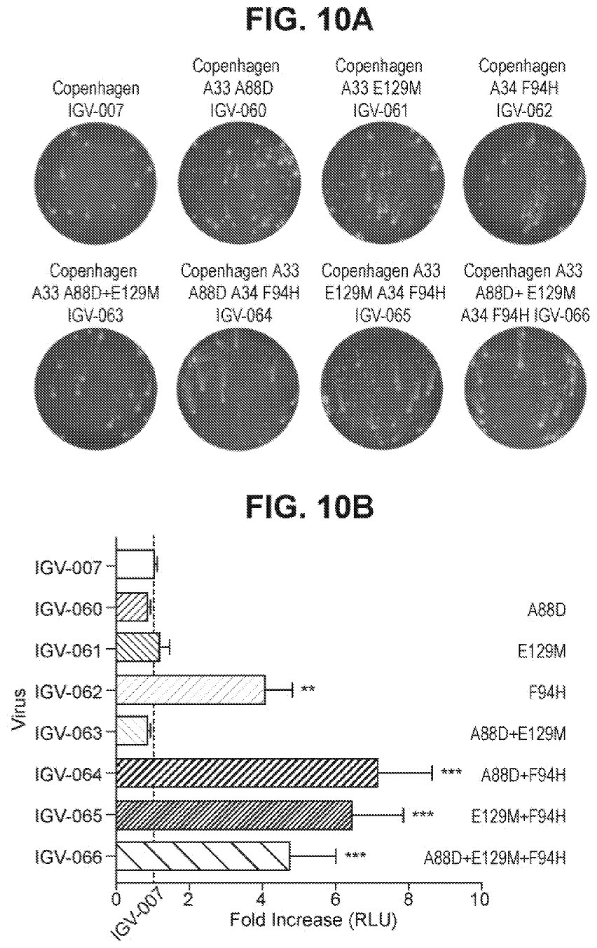

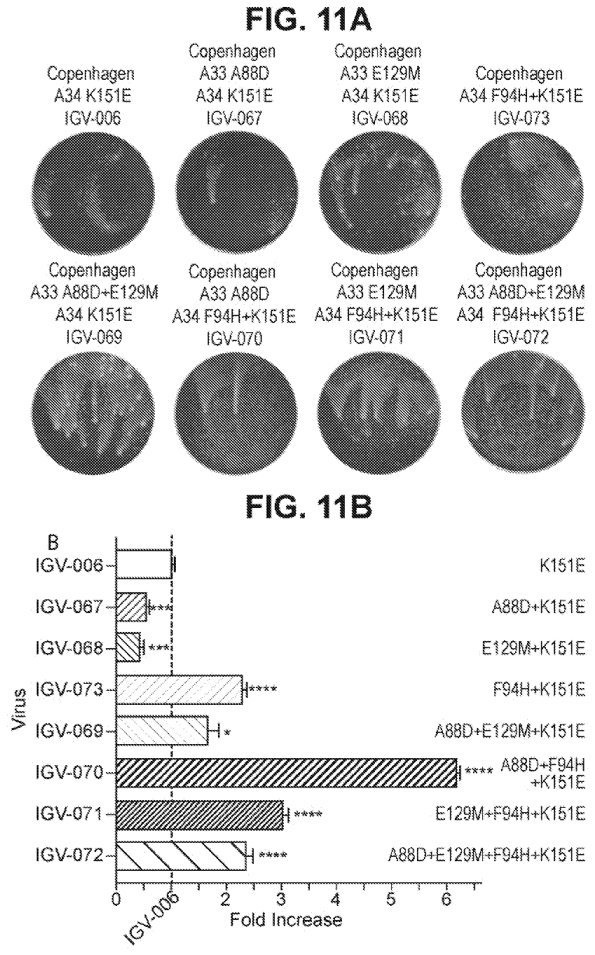

The A33 and A34 mutations identified in IGV-051 and IGV-52 viruses were used to engineer new viruses using IGV-006 and IGV-007 as viral backbones in all possible combinations, as described in Example 1. The evaluation of all single substitutions and combinations of the A88D, E129M, F94H, and K151E mutations enabled the identification of additional combinations and substitutions that conferred spreading advantages. The impact of the single or multiple substitutions on EEV production and viral spreading was assessed in vitro using BSC-40 (African green monkey kidney) and U-2 OS (human osteosarcoma) cells lines, as described in Example 1. Copenhagen vaccinia viruses containing the wild-type A33 and A34 sequences (IGV-007), a K151E substitution in A34 (IGV-006; a known mutation to increase virus spreading and EEV production), an A88D substitution in A33 (IGV-060), an E129M substitution in A33 (IGV-061), an F94H substitution in A34 (IGV-062), the A88D and E129M substitutions in A33 (IGV-063), an A88D substitution in A33 and an F94H substitution in A34 (IGV-064), an E129M substitution in A33 and an F94H substitution in A34 (IGV-065), the A88D and E129M substitutions in A33 and an F94H substitution in A34 (IGV-066), an A88D substitution in A33 and a K151E substitution in A34 (IGV-067), an E129M substitution in A33 and a K151E substitution in A34 (IGV-068), the A88D and E129M substitutions in A33 and a K151E substitution in A34 (IGV-069), an A88D substitution in A33 and the F94H and K151E substitutions in A34 (IGV-070), an E129M substitution in A33 and the F94H and K151E substitutions in A34 (IGV-071), the A88D and E129M substitutions in A33 and the F94H and K151E substitutions in A34 (IGV-072), or the F94H and K151E substitutions in A34 (IGV-073) were generated and manufactured as described in Example 1. The comet assay was performed as described in Example 1. In this assay, combinations of substitutions that included F94H consistently yielded longer and more widespread comet tails (FIG. 10A: IGV-062, IGV-064, IGV-065, IGV-066 vs IGV-007). Conversely, the presence of A88D or E129M appeared to subtly, yet appreciably affect the size of the original comets (FIG. 10A: IGV-066, IGV-064, IGV-061 vs IGV-007). The combination of A88D and F94H is synergistic: slightly reducing plaque size while enhancing the comet tails spreading (FIG. 10A: IGV-60, IGV-62 vs IGV-64). In the presence of K151E, F94H-containing viruses consistently had enhanced comet formation (FIG. 11A: IGV-073 vs IGV-006, IGV-070 vs IGV-067, IGV-071 vs IGV-068, IGV-072 vs IGV-069). The spread of the variants was assessed using the spreading assay described in Example 1. Luciferase activity was compared to luciferase activity of Copenhagen vaccinia virus containing wild-type A33 and A34 sequences (IGV-007) and luciferase activity of Copenhagen vaccinia virus containing a K151E substitution in A34 (IGV-006) and is reported as a fold increase (FIG. 10B, FIG. 11B). Compared to Copenhagen vaccinia viruses containing the wild-type A33 and A34 sequences (IGV-007), an F94H substitution in A34 (IGV-062), an A88D substitution in A33 and an F94H substitution in A34 (IGV-064), an E129M substitution in A33 and an F94H substitution in A34 (IGV-065), and the A88D and E129M substitutions in A33 and an F94H substitution in A34 (IGV-066), resulted in higher luciferase activity, demonstrating that the variant vaccinia viruses are capable of stronger spread (FIG. 10B). Compared to Copenhagen vaccinia virus containing a K151E substitution in A34 (IGV-006), the A88D and E129M substitutions in A33 and a K151E substitution in A34 (IGV-069), an A88D substitution in A33 and the F94H and K151E substitutions in A34 (IGV-070), an E129M substitution in A33 and the F94H and K151E substitutions in A34 (IGV-071), the A88D and E129M substitutions in A33 and the F94H and K151E substitutions in A34 (IGV-072), and the F94H and K151E substitutions in A34 (IGV-073), resulted in higher luciferase activity, demonstrating that the variant vaccinia viruses are capable of stronger spread (FIG. 11B). This study further illustrates that incorporation of mutations to A33 and A34, in particular at the A88 and E129 locations in A33 and the F94 location in A34, either alone or in combination leads to enhanced EEV production and enhanced viral spreading in human cancer cells.

FIG. 10A-10B provide data for different variant vaccinia virus substitutions and combinations of substitutions in the absence of a K151E substitution in A34 on EEV production and viral spreading. A) Representative images of comets formed following infection of BSC-40 cells demonstrate that some variant vaccinia viruses in the absence of the A34 K151E substitution result in longer comet tails, an indication of increased virus spreading and EEV production, compared to Copenhagen vaccinia virus without substitutions in A34 (IGV-007, top left). B) U-2 OS cells were infected with vaccinia virus at MOI 0.6 for 1 hour. Twenty-two hours post-infection, supernatant was collected, clarified, and used to infect a new plate of cells. Luciferase levels (measured as RLU) was determined 15 hours post-secondary infection. Luciferase levels compared to the luciferase levels of Copenhagen vaccinia virus without substitutions in A34 (IGV-007) are reported as a fold increase. A higher luciferase level was observed for most single substitutions and substitution combinations, demonstrating that the variant vaccinia viruses are capable of stronger spread. IGV-007 is Copenhagen (control), IGV-060 is A33 A88D substitution, IGV-061 is A33 E129M substitution, IGV-062 is A34 F94H substitution, IGV-063 is A33 A88D and E129M substitutions, IGV-064 is A33 A88D and A34 F94H substitutions, IGV-065 is A33 E129M and A34 F94H substitutions, IGV-066 is A33 A88D and E129M and A34 F94H substitutions. Error bars indicate SD (n=3). Asterisks indicate statistical significance against Copenhagen vaccinia virus without substitutions in A34 (IGV-007) (**p<0.01, ***p<0.001, ****<0.0001; one-way ANOVA and Dunnett's multiple comparison test).

FIG. 11A-11B provide data for different variant vaccinia virus substitutions and combinations of substitutions in addition to the K151E substitution in A34 on EEV production and viral spreading. A) Representative images of comets formed following infection of BSC-40 cells demonstrate that variant vaccinia viruses in the presence of the A34 K151E substitution result in longer comet tails, an indication of increased virus spreading and EEV production, compared to Copenhagen vaccinia virus containing a K151E substitution in A34 (IGV-006, top left). B) U-2 OS cells were infected with vaccinia virus at MOI 0.33 for 1 hour. Twenty-two hours post-infection, supernatant was collected, clarified, and used to infect a new plate of cells. Luciferase levels (measured as RLU) were determined 15 hours post-secondary infection. Luciferase levels compared to the luciferase levels of Copenhagen vaccinia virus containing a K151E substitution in A34 (IGV-006) are reported as a fold increase. A higher luciferase level was observed for most variant combinations, demonstrating that the variant vaccinia viruses are capable of stronger spread. IGV-006 is Copenhagen A34 K151E substitution (control), IGV-067 is A33 A88D and A34 K151E substitutions, IGV-068 is A33 E129M and A34 K151E substitutions, IGV-073 is A34 F94H and K151E substitutions, IGV-069 is A33 A88D and E129M and A34 K151E substitutions, IGV-070 is A33 A88D and A34 F94H and K151E substitutions, IGV-071 is A33 E129M and A34 F94H and K151E substitutions, IGV-072 is A33 A88D and E129M and A34 F94H and K151E substitutions. Error bars indicate SD (n=3). Asterisks indicate statistical significance against Copenhagen A34 K151E (IGV-006) (*p<0.05, **p<0.01, ***p<0.001, ****<0.0001; one-way ANOVA and Dunnett's multiple comparison test).

### Example 6

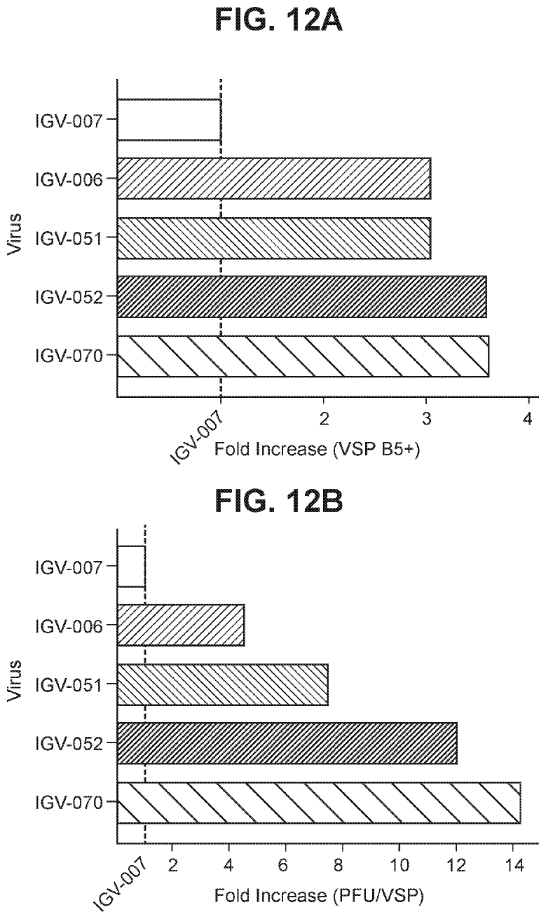

To further assess the substitution variants' impact on EEV total physical particle production and specific infectivity in cancer cells, the number of physical and infectious EEVs produced in HeLa S3 cells (cervical adenocarcinoma) at 24 hours post-infection was determined. Supernatant from infected HeLa S3 cells was collected and purified by cesium chloride (CsCl) gradients to obtain purified EEVs. The infectious EEVs were titered by plaque assay as described in Example 1. The physical number of virion sized particles (VSP) and the percentage of those particles stained with B5 antibody was quantified by Apogee. The viral variants containing the A88D and E129M substitutions in A33 and a K151E substitution in A34 (IGV-051), the F94H and K151E substitutions in A34 (IGV-052), and an A88D substitution in A33 and the F94H and K151E substitutions in A34 (IGV-070) produced a higher percentage of physical EEV particles, measured by antibody staining for the EEV membrane protein B5 when compared to Copenhagen vaccinia virus containing the wild-type A33 and A34 sequences (IGV-007) (FIG. 12A). In addition to the increased physical EEV particles generated, the viral variants (IGV-051, IGV-052 and IGV-070) produced EEVs with increased specific infectivity measured by calculating the infectivity of the VSP divided by PFU, when compared to Copenhagen vaccinia virus containing the wild-type A33 and A34 sequences (IGV-007) and Copenhagen vaccinia virus with a K151E substitution in A34 (IGV-006; a known mutation to increase virus spreading and EEV production) (FIG. 12B). This study further illustrates that incorporation of mutations to A33 and A34, in particular at the A88 and E129 locations in A33 and the F94 location in A34 in combination with the K151E substitution in A34, leads to enhanced physical EEV production with increased specific infectivity.

FIG. 12A-12B provide data for different variants of vaccinia virus in production of physical EEVs and specific infectivity in HeLa S3 (cervical adenocarcinoma) cell line. HeLa S3 cells (cervical adenocarcinoma) were infected with different vaccinia virus variants. Twenty-four hours post-infection, viral particles from the supernatant were collected and purified by CsCl gradient. A gradient band corresponding to EEV density was recovered. The total number of viral sized particles (VSP), the percentage of B5+ VSP, and the infectious virus produced in the supernatant were quantified. A) The percentage of B5+ VSP (indicator of presence of EEV envelope protein) were quantified by Apogee. The data is reported as the fold increase of each recombinant viral variant against Copenhagen vaccinia virus containing the wild-type A33 and A34 sequences (IGV-007). B) The specific infectivity of EEV particles is calculated as the ratio of infectious virus per physical VSP in the supernatant. The data is reported as a fold increase in comparison with Copenhagen vaccinia virus containing the wild-type A33 and A34 sequences (IGV-007). The increased amount of physical and infectious EEV viral particles compared to Copenhagen vaccinia virus containing the wild-type A33 and A34 sequences and Copenhagen vaccinia virus containing a K151E substitution in A34 (IGV-006) demonstrates that the variant vaccinia viruses increase the production of EEVs as well as their specific infectivity. IGV-007 is Copenhagen (control), IGV-006 is Copenhagen A34 K151E substitution (control), IGV-051 is A33 A88D and E129M and A34 K151E substitutions, IGV-052 is A34 F94H and K151E substitutions, IGV-070 is A33 A88D and A34 F94H and K151E substitutions.

### Example 7

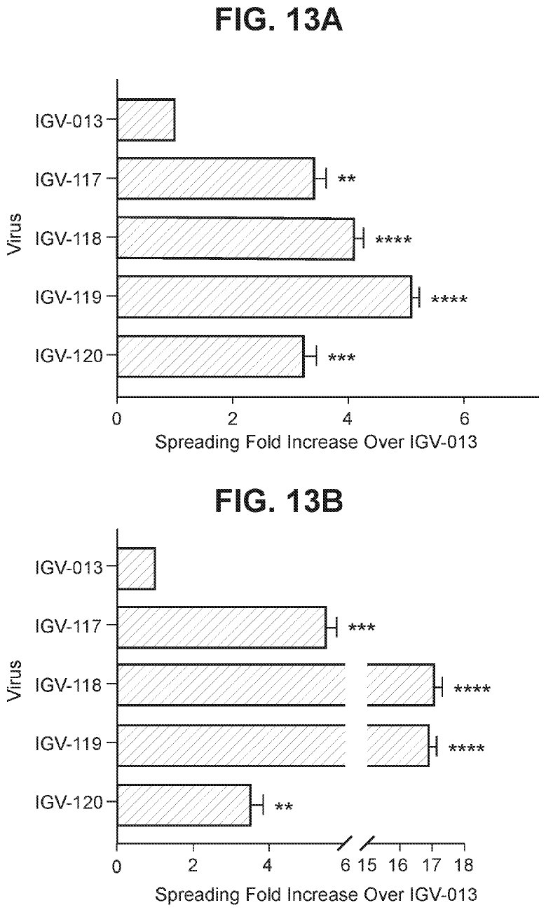

The impact of the viral variants on EEV production and viral spreading in a Western Reserve (WR) vaccinia virus strain was assessed in vitro using RK-13 (rabbit kidney) and VERO-B4 (African green monkey kidney) cell lines. WR vaccinia virus containing the wild-type A33 and A34 sequences (IGV-013), a K151E substitution in A34 (IGV-117; a known mutation to increase virus spreading and EEV production), the F94H and K151E substitutions in A34 (IGV-118), the A88D substitution in A33 and the F94H and K151E substitutions in A34 (IGV-119), and the A88D and E129M substitutions in A33 and the F94H and K151E substitutions in A34 (IGV-120) were generated and manufactured as described in Example 1. The spread of the variants was assessed using the spreading assay described in Example 1. When compared to WR vaccinia virus containing the wild-type A33 and A34 sequences (IGV-013), the variant vaccinia viruses generated a higher degree of relative light units (RLU), indicating enhanced viral spreading (FIG. 13). These studies illustrate that incorporation of mutations to A33 and A34, in particular at the A88 and E129 locations in A33 and the F94 location in A34 in combination with the K151E substitution in A34, leads to enhanced EEV production and therefore virus spreading in other vaccinia virus strains like WR.

FIG. 13A-13B provide data for different variant vaccinia virus substitutions in combination with a K151E substitution in A34 on a Western Reserve strain on EEV production and viral spreading. A) RK-13 cells were infected with vaccinia virus at MOI 50 for 1 hour. Twenty-four hours post-infection, supernatant was collected, clarified, and used to infect a new plate of cells. Luciferase levels (measured as RLU) were determined 24 hours post-secondary infection. Luciferase levels of the variants compared to the luciferase levels of Western Reserve vaccinia virus (IGV-013) is reported as a fold increase. B) VERO-B4 cells were infected with vaccinia virus at MOI 50 for 1 hour. Twenty-four hours post-infection, supernatant was collected, clarified, and used to infect a new plate of cells. Luciferase levels (measured as RLU) were determined 24 hours post-secondary infection. Luciferase levels of the variants compared to the luciferase levels of Western Reserve vaccinia virus (IGV-013) is reported as a fold increase. Higher luciferase levels were observed for all variant combinations, demonstrating that the variant vaccinia viruses are capable of stronger spread. IGV-013 is Western Reserve with wild type A33 and A34 sequences (control), IGV-117 is Western Reserve A34 K151E substitution (control), IGV-118 is A34 F94H and K151E substitutions, IGV-119 is A33 A88D and A34 F94H and K151E substitutions, IGV-120 is A33 A88D and E129M and A34 F94H and K151E substitutions. Error bars indicate SD (n=3). Asterisks indicate statistical significance against Western Reserve (IGV-013) (*p<0.05, **p<0.01, ***p<0.001, ****<0.0001; one-way ANOVA and Dunnett's multiple comparison test).

### Example 8

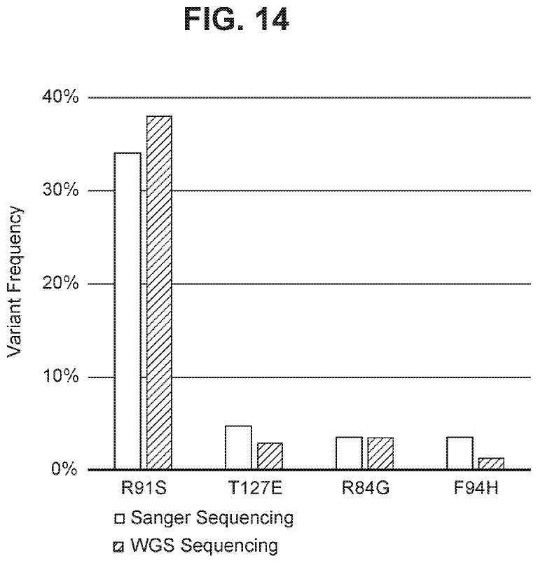

The next selection identified variants capable of enhanced virus spreading and EEV production following infection of HCT 116 colorectal human cancer cells. Four variants were identified that demonstrated an increased frequency within the sequenced population (FIG. 14). Of the four variants, the one containing an R91S substitution to the A34 sequence showed the most enrichment in the final round of selection (FIG. 14).

FIG. 14 provides data on the frequency of specific vaccinia virus variants in the final round of the directed evolution process to identify variants capable of enhanced virus spreading and EEV production following infection and selection on HCT 116 human colorectal cancer cells. A representative fraction of the library was sequenced following the final round of selection using whole genome sequencing (WGS) or Sanger sequencing of individual plaques. The frequency of the four most prevalent variants, expressed as a percentage of the total population, increased significantly over the course of the selection. Enrichment for A34 variants containing R91S, T127E, R84G, or F94H substitutions demonstrates that these substitutions increase the ability of vaccinia virus to spread and produce EEV following infection of human cancer cells.

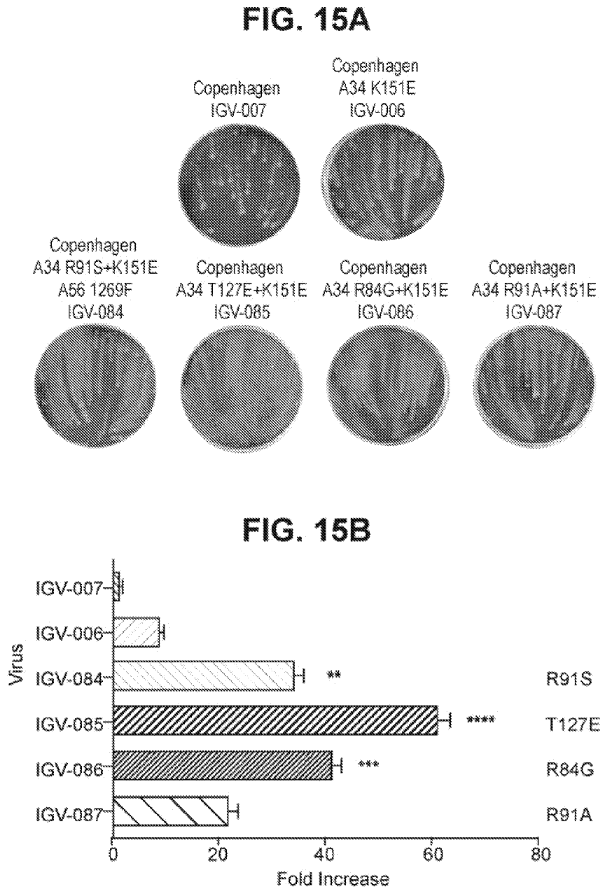

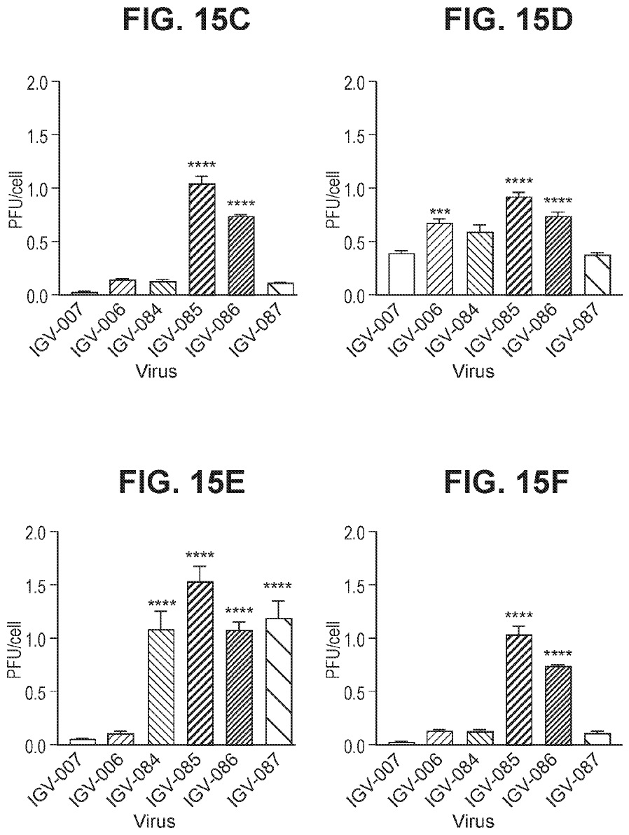

The impact of the substitution variants on EEV production and viral spreading was assessed in vitro using BSC-40 (African green monkey kidney) and U-2 OS (human osteosarcoma) cell lines. Copenhagen vaccinia viruses containing the wild-type A33 and A34 sequences (IGV-007), a K151E substitution in A34 (IGV-006; a known mutation to increase virus spreading and EEV production), R91S and K151E substitutions in A34 and I269F substitution in A56 (IGV-084), T127E and K151E substitutions in A34 (IGV-085), R84G and K151E substitutions in A34 (IGV-086), or R91A and K151E substitutions in A34 (IGV-087) were generated and manufactured as described in Example 1. The comet assay was performed as described in Example 1. The presence of longer comet tails (FIG. 15A) for the variant vaccinia viruses compared to Copenhagen vaccinia virus containing the wild-type A34 sequence indicates more virus spreading and increased production of the EEV form by the variant vaccinia viruses. The spread of the variants was assessed using the spreading assay described in Example 1. Compared to Copenhagen vaccinia virus containing a K151E substitution in A34 (IGV-006), the R91S and K151E substitutions in A34 and I269F substitution in A56 (IGV-084), T127E and K151E substitutions in A34 (IGV-085), or R84G and K151E substitutions in A34 (IGV-086), or R91A and K151E substitutions in A34 (IGV-087), resulted in higher luciferase levels, demonstrating that the variant vaccinia viruses are capable of stronger spread (FIG. 15B). The impact of the substitution variants on infectious virus particle production in the supernatant (EEV production) in different human colorectal cancer cells lines was assessed in vitro using HCT 116 cells (colorectal carcinoma), HT-29 cells (colorectal adenocarcinoma), Colo205 cells (colorectal adenocarcinoma), and SW-620 cells (colorectal adenocarcinoma). All cell lines listed above were infected with a MOI of 3 and washed thoroughly 3 times (except for Colo205 cells which were washed once) after 1 hour of virus adsorption. At 24 hours post-infection, the supernatant was collected and the number of infectious virus particles produced (potential EEV) were determined by performing a plaque assay as described in Example 1. In all four cell lines, the substitution variants containing a combination of a K151E substitution in A34 and the T127 substitution in A34 (IGV-085), or the R84G substitution in A34 (IGV-086) produced a higher number of infectious virus particles in the supernatant, suggesting an increase in EEV particle formation (FIG. 15C-15F). This study further illustrates that incorporation of mutations to A34, in particular at the R84, R91, and T127 locations in A34 in combination with the K151E substitution in A34 and I269F substitution in A56 leads to enhanced EEV production and enhanced viral spreading in human colorectal cancer cells.

FIG. 15A-15F provides data on virus spreading and EEV production of additional vaccinia virus variants containing A34 substitutions. A) Representative images of plaques formed following infection of BSC-40 cells demonstrate that the variant vaccinia viruses result in longer comet tails, an indication of better spreading and enhanced EEV production, compared to Copenhagen vaccinia virus containing the wild-type A33 and A34 sequences (top left) and Copenhagen vaccinia virus containing a K151E substitution in A34 (a known mutation to increase virus spreading and EEV production; top right). B) A two-stage infectivity assay to measure viral spreading, in which U-2 OS cells were first infected with equal multiplicities of infection (MOI) of vaccinia virus, then supernatant was collected 22 hours post-infection and used to infect a new plate of U-2 OS cells. Luciferase expressed from the virus is measured at 15 hours post infection, and increased levels of luciferase correlate with higher rates of infection. Thus, higher luciferase is indicative of increased spreading. Data is provided as a fold increase compared to luciferase expression in IGV-006 (Copenhagen vaccinia virus containing a K151E substitution in A34). Vaccinia virus variants containing R91S and K151E substitutions in A34 and I269F substitution in A56 (IGV-084), T127E and K151E substitutions in A34 (IGV-085), R84G and K151E substitutions in A34 (IGV-086), or R91A and K151E substitutions in A34 (IGV-087) show over 2.5-fold increase in viral spreading compared to a Copenhagen virus with a K151E substitution in A34 (IGV-006), which could lead to improved oncolytic activity in cancer. Quantification of the infectious virus in the supernatant 24 hours post-infection of C) HCT 116 (colorectal carcinoma), D) HT-29 (colorectal adenocarcinoma), E) Colo205 (colorectal adenocarcinoma), and F) SW-620 (colorectal adenocarcinoma) cell lines. Cell lines were infected with vaccinia virus at a MOI of 3. Twenty-four hours post-infection, the number of infectious virus particles produced in the supernatant (EEVs) were determined by plaque assay. IGV-007 is Copenhagen (control), IGV-006 is Copenhagen A34 K151E substitution, IGV-084 is Copenhagen A34 R91S and K151E substitutions and A56 I269F substitution, IGV-085 is Copenhagen A34 T127E and K151E substitutions, IGV-086 is Copenhagen A34 R84G and K151E substitutions, IGV-087 is Copenhagen A34 R91A and K151E substitutions. Error bars indicate SD (n=3). Asterisks indicate statistical significance against Copenhagen vaccinia virus with K151E substitution in A34R gene (IGV-006) (**p<0.01, ***p<0.001, ****<0.0001; ordinary one-way ANOVA followed by Tukey's multiple comparison test).

### Example 9

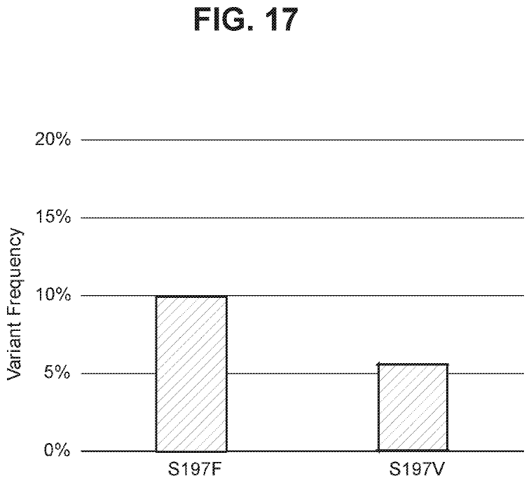

The next selection identified variants capable of enhanced virus spreading and EEV production following infection of Colo 205 colorectal human cancer cells. Two variants were identified that demonstrated an increased frequency within the sequenced population (FIG. 17). Of the two variants, the one containing an S197F substitution to the B5 sequence showed the most enrichment in the final round of selection (FIG. 17).

FIG. 17 provides data on the frequency of specific vaccinia virus variants in the final round of the directed evolution process to identify variants capable of enhanced virus spreading and EEV production following infection and selection on Colo 205 human colorectal cancer cells. A representative fraction of the library was sequenced following the final round of selection using amplicon next-generation sequencing (NGS). The frequency of the two most prevalent variants, expressed as a percentage of the total population, increased significantly over the course of the selection. Enrichment for B5 variants containing S197F, or S197V substitutions demonstrates that these substitutions increase the ability of vaccinia virus to spread and produce EEV following infection of human cancer cells.

### Example 10

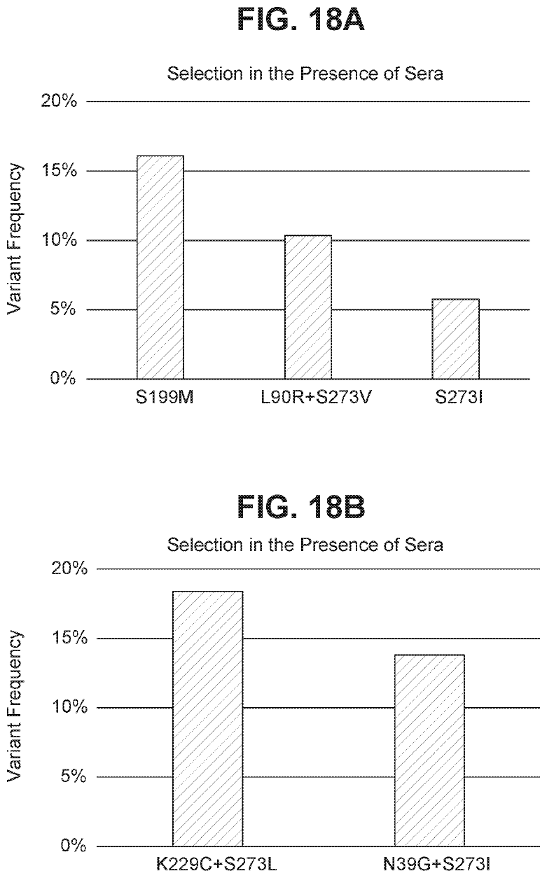

The following selections identified variants capable of enhanced virus spreading and EEV production following infection of MDA-MB-231 breast cancer cells in the absence or presence of serum from donors vaccinated with vaccinia virus. Five total variants were identified, three in the absence of serum and two in the presence of serum, that demonstrated an increased frequency within the sequenced population (FIG. 18).

FIG. 18A-18B provides data on the frequency of specific vaccinia virus variants in the final round of the directed evolution process to identify variants capable of enhanced virus spreading and EEV production following infection of MDA-MB-231 human breast cancer cells in the absence or presence of serum from donors vaccinated with vaccinia virus. A representative fraction of the library was sequenced following the final round of selection using either whole genome sequencing (WGS) or Sanger sequencing of individual plaques. The frequency of the five most prevalent variants, expressed as a percentage of the total population, increased significantly over the course of the selection. Enrichment for B5 variants containing N39G and S273I substitutions, an S199M substitution, L90R and S273V substitutions, K229C and S273L substitutions, or an S273I substitution demonstrates that these substitutions increase the ability of vaccinia virus to enhance virus spreading and produce EEVs following infection of human breast cancer cells.

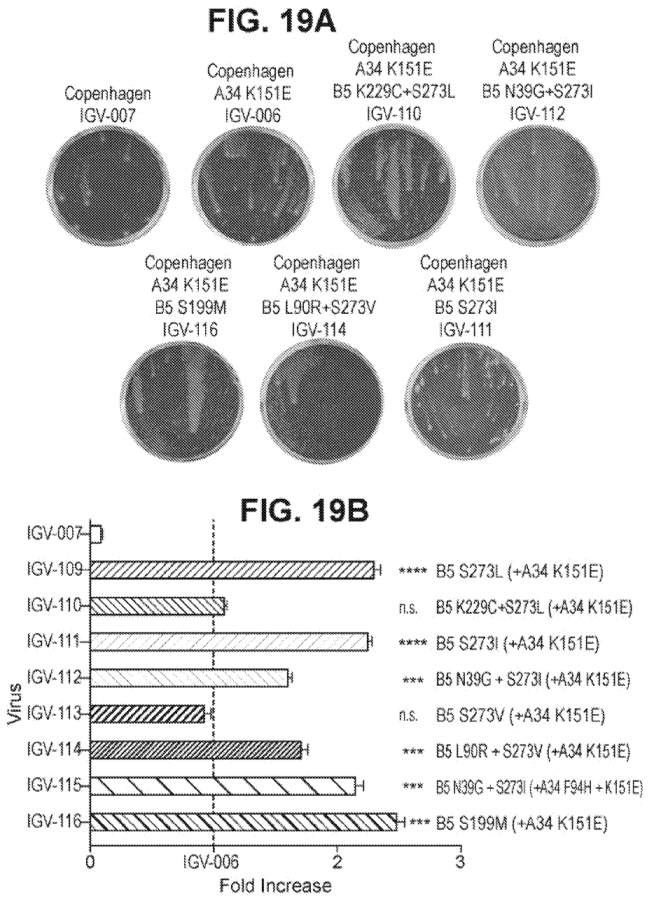

The impact of the substitution variants on EEV production and viral spreading was assessed in vitro using BSC-40 (African green monkey kidney) and U-2 OS (human osteosarcoma) cell lines. Copenhagen vaccinia viruses containing the wild-type A33, A34, and B5 sequences (IGV-007), a K151E substitution in A34 (IGV-006; a known mutation to increase virus spreading and EEV production), the K229C and S273L substitutions in B5 (IGV-110), the N39G and S273I substitutions in B5 (IGV-112), the S199M substitution in B5 (IGV-116), the L90R and S273V substitutions in B5 (IGV-114), or the S273I substitution in B5 (IGV-111) were generated and manufactured as described in Example 1. The comet assay was performed as described in Example 1. The presence of longer comet tails (FIG. 19A) for the variant vaccinia viruses compared to Copenhagen vaccinia virus containing the wild-type B5 sequence indicates that more of the EEV form of the virus is being produced by the variant vaccinia viruses. The spread of the variants was assessed using the spreading assay described in Example 1. Compared to Copenhagen vaccinia virus containing a K151E substitution in A34 alone (IGV-006), the combination of a K151E substitution in A34 and most substitutions in B5 resulted in higher luciferase activity, demonstrating that the variant vaccinia viruses are capable of stronger spread (FIG. 19B). In particular, the combination of a K151E substitution in A34 with an S273L substitution in B5 (IGV-109), an S273I substitution in B5 (IGV-111), N39G and S273I substitutions in B5 (IGV-112), L90R and S273V substitutions in B5 (IGV-114), or an S199M substitution in B5 (IGV-116) lead to significantly enhanced spread. This study further illustrates that incorporation of mutations to B5, either alone or in combination leads to enhanced EEV production and enhanced viral spreading in cancer cells.

FIG. 19A-19B provide data on virus spreading and EEV production of vaccinia virus variants containing B5 substitutions. A) Representative images of comets formed following infection of BSC-40 cells demonstrate that variant vaccinia viruses with a combination of substitutions in B5 and the A34 K151E substitution result in longer comet tails, an indication of increased EEV production, compared to Copenhagen vaccinia virus (IGV-007, top left). B) U-2 OS cells were infected with vaccinia virus at MOI 0.33 for 1 hour. Twenty-two hours post-infection, supernatant was collected and used to infect a new plate of cells. Luciferase levels (measured as RLU) were determined 15 hours post-secondary infection. Luciferase levels of the viral variants compared to the luciferase levels of Copenhagen vaccinia virus containing a K151E substitution in A34 (IGV-006) are reported as fold increase. Higher luciferase activity was observed for most variant combinations, demonstrating that the variant vaccinia viruses are capable of stronger spread. IGV-007 is Copenhagen (control), IGV-006 is Copenhagen A34 K151E substitution (control), IGV-109 is B5 S273L and A34 K151E substitutions, IGV-110 is B5 K229C and S273L and A34 K151E substitutions, IGV-111 is B5 S273I and A34 K151E substitutions, IGV-112 is B5 N39G and S273I and A34 K151E substitutions, IGV-113 is B5 S273V and A34 K151E substitutions, IGV-114 is B5 L90R and S273V and A34 K151E substitutions, IGV-115 is B5 N39G and S273I and A34 F94H and K151E substitutions, IGV-116 is B5 S199M and A34 K151E substitutions. Error bars indicate SD (n=3). Asterisks indicate statistical significance against Copenhagen A34R_K151E (IGV-006) (*p<0.05, **p<0.01, ***p<0.001, ****<0.0001; one-way ANOVA and Dunnett's multiple comparison test).

### Example 11

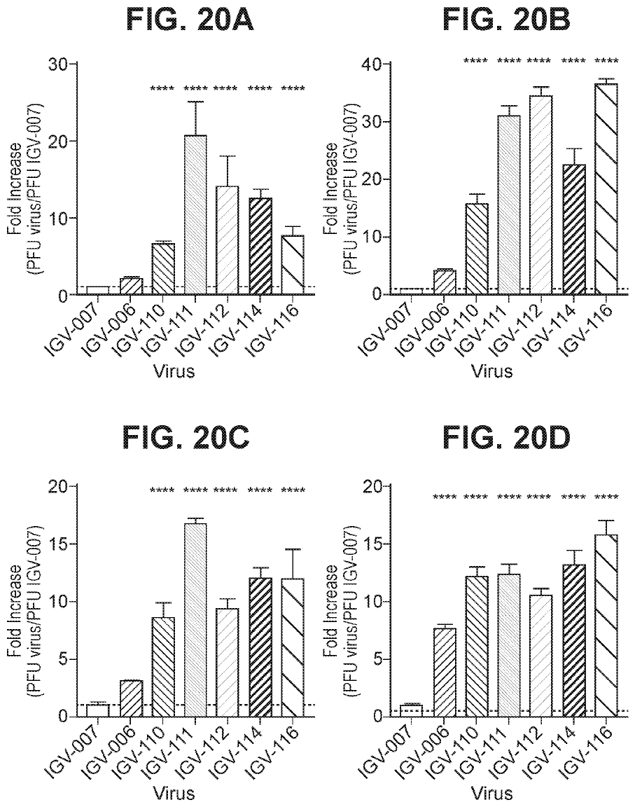

The impact of the substitution variants on infectious virus particle production in the supernatant (EEV production) in different human cancer cells lines was assessed in vitro using MDA-MB-231 cells (breast adenocarcinoma), MCF7 cells (breast adenocarcinoma), T47D cells (breast carcinoma), and HCT 116 cells (colorectal carcinoma). Copenhagen vaccinia viruses containing the wild-type A33, A34, and B5 sequences (IGV-007), a K151E substitution in A34 (IGV-006; a known mutation to increase virus spreading and EEV production), and vaccinia virus variants containing a combination of a K151E substitution in A34 and the K229C and S273L substitutions in B5 (IGV-110), the S273I substitution in B5 (IGV-111), the N39G and S273I substitutions in B5 (IGV-112), the L90R and S273V substitutions in B5 (IGV-114), or the S199M substitution in B5 (IGV-116) were generated and manufactured as described in Example 1. All cell lines listed above were infected with a MOI of 3 and washed thoroughly 3 times after 1 hour of virus adsorption. At 24 hours post-infection, the supernatant was collected and the number of infectious virus (EEV) particles produced was determined by performing a plaque assay as described in Example 1. In all four cell lines, the substitution variants containing a combination of a K151E substitution in A34 and the K229C and S273L substitutions in B5 (IGV-110), the S273I substitution in B5 (IGV-111), the N39G and S273I substitutions in B5 (IGV-112), the L90R and S273V substitutions in B5 (IGV-114), or the S199M substitution in B5 (IGV-116) produced a higher number of infectious virus particles in the supernatant, suggesting an increase in EEV particle formation (FIG. 20A-20D). This study demonstrates that incorporation of mutations to B5 leads to enhanced infectious EEV production in different cancer cell lines.

FIG. 20A-20D provide data on vaccinia virus infectious virions in the supernatant (potential EEVs) in representative human cancer cell lines. A) MDA-MB-231 cells (breast adenocarcinoma), B) MCF7 cells (breast adenocarcinoma), C) T47D cells (breast carcinoma), and D) HCT 116 cells (colorectal carcinoma) were infected with vaccinia virus at a MOI of 3. Twenty-four hours post-infection, the number of infectious virus particles produced in the supernatant (EEVs) were determined by plaque assay. The fold increase of infectious virus particles in the supernatant produced in each cancer cell line was calculated for each virus variant compared to a Copenhagen virus containing the wild-type A33, A34, and B5 sequences (IGV-007). There is a significant increase of infectious viral particles for vaccinia virus variants compared to Copenhagen containing the wild-type A33, A34, and B5 sequences in all cancer cell lines tested. IGV-007 is Copenhagen (control), IGV-006 is Copenhagen A34 K151E substitution (control), IGV-110 is B5 K229C and S273L and A34 K151E substitutions, IGV-111 is B5 S273I and A34 K151E substitutions, IGV-112 is B5 N39G and S273I and A34 K151E substitutions, IGV-114 is B5 L90R and S273V and A34 K151E substitutions, IGV-116 is B5 S199M and A34R K151E substitutions. Error bars indicate SD (n=3). Asterisks indicate statistical significance against Copenhagen vaccinia virus containing the wild-type A33, A34, and B5 sequences (IGV-007) (*p<0.5, ****<0.0001; one-way ANOVA and Dunnett's multiple comparison test).

### Example 12

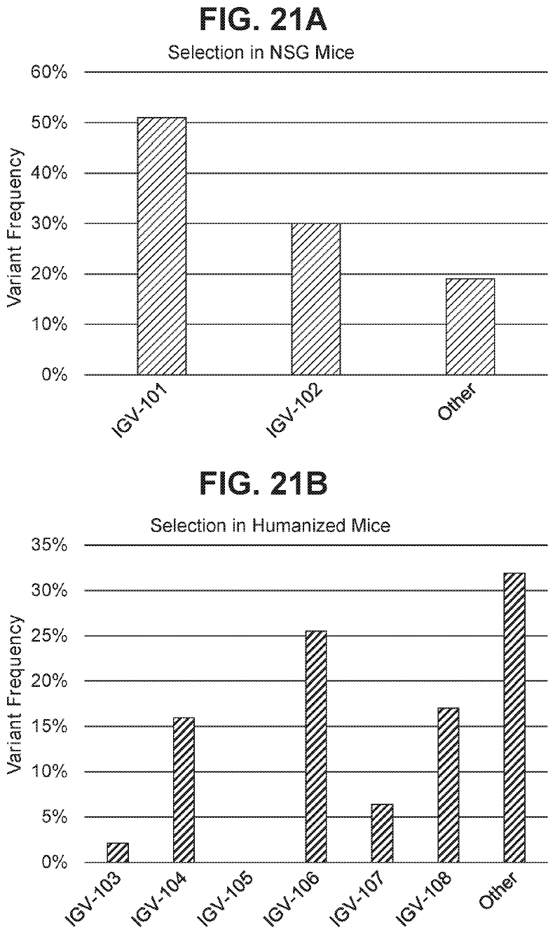

In vivo selections performed in mouse cancer models identified variants capable of enhanced spreading and EEV production following infection of mice containing human patient-derived cancer cells with vaccinia virus. Eight total variants were identified, two using a patient-derived xenograft (PDX) mouse bilateral model of colorectal cancer and six using a patient-derived xenograft (PDX) mouse bilateral model of colorectal cancer that also contained a humanized blood system, that demonstrated an increased frequency within the sequenced population (FIG. 21).

FIG. 21A-21B provide data on the frequency of specific vaccinia virus variants in the directed evolution process to identify variants capable of enhanced spread in vivo. A representative fraction of the library was sequenced following the final round of selection using whole genome sequencing (WGS) or Sanger sequencing of individual plaques (plaque sequencing). The frequency of the eight most prevalent variants, expressed as a percentage of the total population, increased significantly over the course of the selection. Enrichment for B5 variants containing multiple substitutions within or around the stalk region and A33/A34 variants demonstrates that these substitutions increase the ability of vaccinia virus to enhance spreading following infection of human cancer cells in vivo.

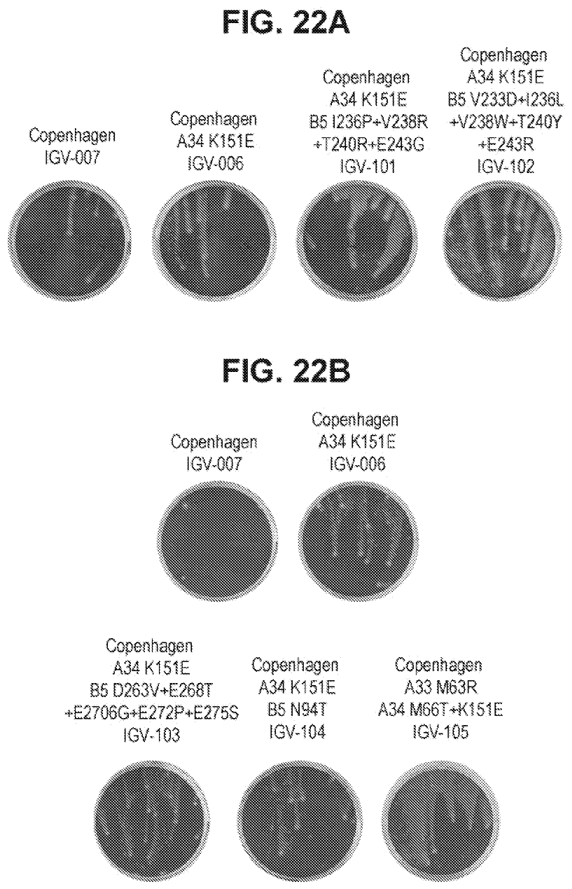

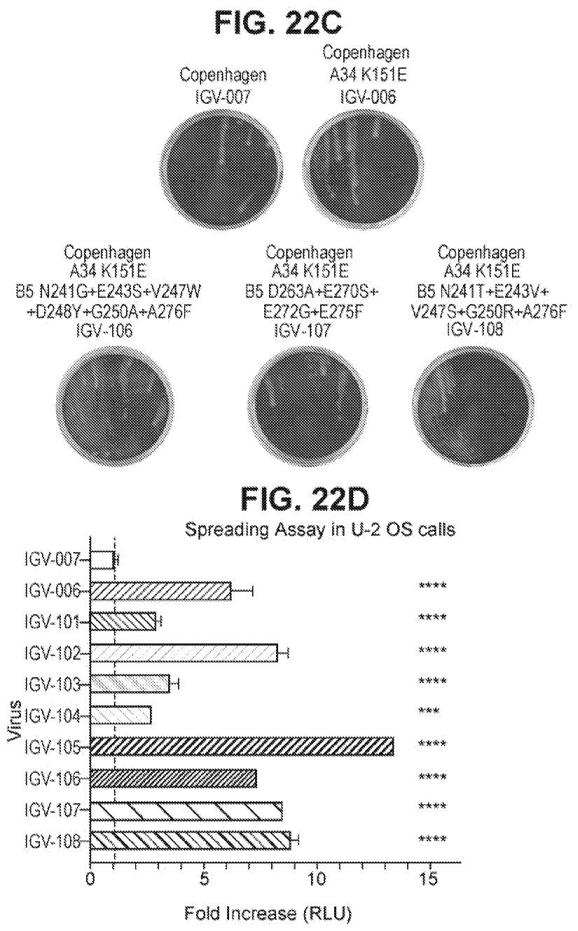

The impact of the substitution variants on EEV production and viral spreading was assessed in vitro using BSC-40 (African green monkey kidney) and U-2 OS (human osteosarcoma) cell lines. Copenhagen vaccinia viruses containing the wild-type A33, A34, and B5 sequences (IGV-007), a K151E substitution in A34 (IGV-006; a known mutation to increase virus spreading and EEV production), and variants containing the combination of a K151E substitution in A34 with I236P, V238R, T240R, and E243G substitutions in B5 (IGV-101), V233D, I236L, V238W, T240Y, and E243R substitutions in B5 (IGV-102), D263V, E268T, E270G, E272P, and E275S substitutions in B5 (IGV-103), an N94T substitution in B5 (IGV-104), an M63R substitution in A33 and an M66T substitution in A34 (IGV-105), N241G, E243S, V247W, D248Y, G250A, and A276F substitutions in B5 (IGV-106), D263A, E270S, E272G, and E275F substitutions in B5 (IGV-107), or N241T, E243V, V247S, G250R, and A276F substitutions in B5 (IGV-108) were generated and manufactured as described in Example 1. The comet assay was performed as described in Example 1. The presence of longer comet tails (FIG. 22A-22C) for the variant vaccinia viruses compared to Copenhagen vaccinia virus containing the wild-type A33, A34, and B5 sequences indicates that better spreading and more of the EEV form of the virus is being produced by the variant vaccinia viruses. The spread of the variants was assessed using the spreading assay described in Example 1. Compared to Copenhagen vaccinia virus containing the wild-type A33, A34, and B5 sequences (IGV-007), the combination of a K151E substitution in A34 and substitutions in A33/A34 or B5 resulted in higher luciferase levels, demonstrating that the variant vaccinia viruses are capable of stronger spread (FIG. 22D). In particular, the combination of a K151E substitution in A34 with V233D, I236L, V238W, T240Y, and E243R substitutions in B5 (IGV-102), an M63R substitution in A33 and an M66T substitution in A34 (IGV-105), N241G, E243S, V247W, D248Y, G250A, and A276F substitutions in B5 (IGV-106), D263A, E270S, E272G, and E275F substitutions in B5 (IGV-107), or N241T, E243V, V247S, G250R, and A276F substitutions in B5 (IGV-108) led to significantly enhanced spread. This study further illustrates that incorporation of mutations to A33, A34, or B5, either alone or in combination leads to enhanced EEV production and enhanced viral spreading in cancer cells.

FIG. 22A-22D provide data on virus spreading and EEV production of additional vaccinia virus variants containing A33/A34 or B5 substitutions. A) Representative images of comets formed following infection of BSC-40 cells demonstrate that variant vaccinia viruses with a combination of substitutions in B5 and the A34 K151E substitution result in longer comet tails, an indication of increased spreading and EEV production, compared to Copenhagen vaccinia virus (IGV-007, left). B) Representative images of comets formed following infection of BSC-40 cells demonstrate that variant vaccinia viruses with a combination of substitutions in A33/A34 or B5 and the A34 K151E substitution result in longer comet tails, an indication of increased spreading and EEV production, compared to Copenhagen vaccinia virus (IGV-007, top left). C) Representative images of comets formed following infection of BSC-40 cells demonstrate that variant vaccinia viruses with a combination of substitutions in B5 and the A34 K151E substitution result in longer comet tails, an indication of increased spreading and EEV production, compared to Copenhagen vaccinia virus (IGV-007, top left). D) U-2 OS cells were infected with vaccinia virus at a MOI of 1 for 1 hour. Twenty-two hours post-infection, supernatant was collected and used to infect a new plate of cells. Luciferase expression level (measured as RLU) was determined 15 hours post-secondary infection. Luciferase expression level of the variants compared to the luciferase expression level of Copenhagen vaccinia virus containing wild-type A33, A34, and B5 sequences (IGV-007) is reported as a fold increase. Higher luciferase levels were observed for all variant combinations, demonstrating that the variant vaccinia viruses are capable of stronger spread. IGV-007 is Copenhagen (control), IGV-006 is Copenhagen A34 K151E substitution (control), IGV-101 is B5 I236P, V238R, T240R, and E243G and A34 K151E substitutions, IGV-102 is B5 V233D, I236L, V238W, T240Y, and E243R and A34 K151E substitutions, IGV-103 is B5 D263V, E268T, E270G, E272P, and E275S and A34 K151E substitutions, IGV-104 is B5 N94T and A34 K151E substitutions, IGV-105 is A33 M63R and A34 M66T and K151E substitutions, IGV-106 is B5 N241G, E243S, V247W, D248Y, G250A, and A276F and A34 K151E substitutions, IGV-107 is B5 D263A, E270S, E272G, and E275F and A34 K151E substitutions, and IGV-108 is B5 N241T, E243V, V247S, G250R, and A276F and A34 K151E substitutions. Error bars indicate SD (n=3). Asterisks indicate statistical significance against Copenhagen (IGV-007) (*p<0.05, **p<0.01, ***p<0.001, ****<0.0001; one-way ANOVA and Dunnett's multiple comparison test).

A summary of all vaccinia virus substitution variants identified through directed evolution is provided in Table 2.

TABLE 2

| | A33 | A34 | B5 | A56 | |
|---|---|---|---|---|---|
| Virus ID | Substitution | Substitution | Substitution | Substitution | Tumor |
| Number | (s) | (s) | (s) | (s) | Target |
| IGV-052/ | | F94H + K151E | | | colorectal, |
| IGV-073 | | | | | breast, lung |
| IGV-051/ | A88D + E129M | K151E | | | colorectal, |
| IGV-069 | | | | | breast, lung |
| IGV-060 | A88D | | | | colorectal, |
| | | | | | breast, lung |
| IGV-061 | E129M | | | | colorectal, |
| | | | | | breast, lung |
| IGV-062 | | F94H | | | colorectal, |
| | | | | | breast, lung |
| IGV-063 | A88D + E129M | | | | colorectal, |
| | | | | | breast, lung |
| IGV-064 | A88D | F94H | | | colorectal, |
| | | | | | breast, lung |
| IGV-065 | E129M | F94H | | | colorectal, |
| | | | | | breast, lung |
| IGV-066 | A88D + E129M | F94H | | | colorectal, |
| | | | | | breast, lung |
| IGV-067 | A88D | K151E | | | colorectal, |
| | | | | | breast, lung |
| IGV-068 | E129M | K151E | | | colorectal, |
| | | | | | breast, lung |
| IGV-070 | A88D | F94H + K151E | | | colorectal, |
| | | | | | breast, lung |
| IGV-071 | E129M | F94H + K151E | | | colorectal, |
| | | | | | breast, lung |
| IGV-072 | A88D + E129M | F94H + K151E | | | colorectal, |
| | | | | | breast, lung |
| IGV-084 | | R91S + K151E | | I269F | colorectal |
| IGV-085 | | T127E + K151E | | | colorectal |
| IGV-086 | | R84G + K151E | | | colorectal |
| IGV-087 | | R91A + K151E | | | colorectal |
| IGV-101 | | K151E | I236P + V238R + | | colorectal |
| | | | T240R + E243G | | |
| IGV-102 | | K151E | V233D + I236L + | | colorectal |
| | | | V238W + T240Y + | | |
| | | | E243R | | |
| IGV-103 | | K151E | B5R | | colorectal |
| | | | D263V + E268T + | | |
| | | | E270G + E272P + | | |
| | | | E275S | | |
| IGV-104 | | K151E | N94T | | colorectal |
| IGV-105 | M63R | M66T + K151E | | | colorectal |
| IGV-106 | | K151E | N241G + E243S + | | colorectal |
| | | | V247W+ D248Y + | | |
| | | | G250A + A276F | | |
| IGV-107 | | K151E | D263A + E270S+ | | colorectal |
| | | | E272G + E275F | | |
| IGV-108 | | K151E | N241T + E243V + | | colorectal |
| | | | V247S + G250R + | | |
| | | | A276F | | |
| IGV-110 | | K151E | K229C + S273L | | breast |
| IGV-111 | | K151E | S273I | | breast |
| IGV-112 | | K151E | N39G + S273I | | breast |
| IGV-114 | | K151E | L90R + S273V | | breast |
| IGV-115 | | F94H + K151E | N39G + S273I | | breast |
| IGV-116 | | K151E | S199M | | breast |
| — | | K151E | S197F | | colorectal |
| — | | K151E | S197V | | colorectal |

In embodiments that refer to a method of treatment as described herein, such embodiments are also further embodiments for use in that treatment, or alternatively for the manufacture of a medicament for use in that treatment.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A replication-competent, recombinant oncolytic vaccinia virus (OVV), comprising:
a) a nucleotide sequence encoding a variant A33 polypeptide;
b) a nucleotide sequence encoding a variant A34 polypeptide; or
c) a nucleotide sequence encoding a variant A33 polypeptide and a nucleotide sequence encoding a variant A34 polypeptide,
wherein the variant A33 polypeptide and variant A34 polypeptide each provides for enhanced viral spreading or enhanced production of extracellular enveloped virion (EEV), compared to the corresponding wild-type A33 polypeptide and wild-type A34 polypeptide, respectively.

2. An OVV, comprising a nucleotide sequence encoding a variant B5 polypeptide, wherein the variant B5 polypeptide provides for enhanced viral spreading or enhanced production of EEV, compared to the corresponding wild-type B5 polypeptide.

3. The OVV of claim 2, further comprising one or both of:
a) a nucleotide sequence encoding a variant A33 polypeptide; and
b) a nucleotide sequence encoding a variant A34 polypeptide, wherein the variant A33 polypeptide and variant A34 polypeptide each provides for enhanced viral spreading or enhanced production of EEV, compared to the corresponding wild-type A33 polypeptide and wild-type A34 polypeptide, respectively.

4. The OVV of claim 1 or claim 3, wherein the variant A33 polypeptide comprises a substitution of one or more of M63, A88, and E129.

5. The OVV of claim 4, wherein the substitution is one or more of an M63R substitution, an A88D substitution, and an E129M substitution.

6. The OVV of claim 5, wherein the variant A33 polypeptide comprises an A88D substitution and an E129M substitution.

7. The OVV of any one of claims 4-6, wherein the OVV comprises a nucleotide sequence encoding a variant A33 polypeptide.

8. The OVV of claim 1 or claim 3, wherein the variant A34 polypeptide comprises a substitution of 1, 2, 3, 4, 5, or 6 of M66, F94, R84, R91, T127, and K151.

9. The OVV of claim 8, wherein the substitution is one or more of an M66T substitution, an F94H substitution, an R84G substitution, an R91S substitution, an R91A substitution, a T127E substitution, and a K151E substitution.

10. The OVV of claim 9, wherein the variant A34 polypeptide comprises a K151E substitution.

11. The OVV of claim 9, wherein the variant A34 polypeptide comprises an F94H substitution.

12. The OVV of any one of claims 8-11, wherein the vaccinia virus comprises a nucleotide sequence encoding variant A34 polypeptide.

13. The OVV of claim 1 or claim 3, wherein the vaccinia virus comprises a nucleotide sequence encoding a variant A33 polypeptide and a nucleotide sequence encoding a variant A34 polypeptide, wherein the variant A33 polypeptide comprises a substitution of 1, 2, or 3 of M63, A88, and E129; and wherein the variant A34 polypeptide comprises a substitution of 1, 2, 3, 4, 5, or 6 of M66, F94, R84, R91, T127, and K151.

14. The OVV of claim 13, wherein the variant A33 polypeptide comprises 1, or 3 of an M63R substitution, an A88D substitution, and an E129M substitution; and wherein the variant A34 polypeptide comprises 1, 2, 3, 4, 5, 6, or 7 of an M66T substitution, an F94H substitution, an R84G substitution, an R91S substitution, an R91A substitution, a T127E substitution, and K151E substitution.

15. The OVV of claim 14, wherein the variant A34 polypeptide comprises a K151E substitution.

16. The OVV of claim 14, wherein the variant A33 polypeptide comprises an A88D substitution; and wherein the variant A34 polypeptide comprises an F94H substitution.

17. The OVV of claim 14, wherein the variant A33 polypeptide comprises an E129M substitution and wherein the variant A34 polypeptide comprises an F94H substitution.

18. The OVV of claim 14, wherein the variant A33 polypeptide comprises an A88D substitution and an E129M substitution, and wherein the variant A34 polypeptide comprises an F94H substitution.

19. The OVV of claim 14, wherein the variant A33 polypeptide comprises an A88D substitution and an E129M substitution, and wherein the variant A34 polypeptide comprises a K151E substitution.

20. The OVV of claim 14, wherein the variant A33 polypeptide comprises an A88D substitution, and wherein the variant A34 polypeptide comprises an F94H substitution and a K151E substitution.

21. The OVV of claim 14, wherein the variant A33 polypeptide comprises an E129M substitution, and wherein the variant A34 polypeptide comprises an F94H substitution and a K151E substitution.

22. The OVV of claim 14, wherein the variant A33 polypeptide comprises an A88D substitution and an E129M substitution, and wherein the variant A34 polypeptide comprises an F94H substitution and a K151E substitution.

23. The OVV of claim 2 or claim 3, wherein the variant B5 polypeptide comprises 1, 2, 3, 4, or more amino acid substitutions at positions of N39, L90, N94, S199, K229, V233, 1236, V238, T240, N241, E243, V247, D248, G250, D263, E268, E270, D272, S273, D275, and A276.

24. The OVV of claim 23, wherein the variant B5 polypeptide comprises an S197F or an S197V substitution.

25. The OVV of claim 23, wherein the variant B5 polypeptide comprises an S199M substitution.

26. The OVV of claim 23, wherein the variant B5 polypeptide comprises an S273L substitution or an S273I substitution.

27. The OVV of claim 23, wherein the variant B5 polypeptide comprises an N39G substitution.

28. The OVV of claim 23, wherein the variant B5 polypeptide comprises an N94T substitution.

29. The OVV of claim 23, wherein the variant B5 polypeptide comprises an L90R substitution and an S273V substitution.

30. The OVV of claim 23, wherein the variant B5 polypeptide comprises an N39G substitution and an S273I substitution.

31. The OVV of claim 23, wherein the variant B5 polypeptide comprises a K229C substitution and an S273L substitution.

32. The OVV of claim 23, wherein the variant B5 polypeptide comprises an D263A substitution, an E270S substitution, an E272G substitution, and an E275F substitution.

33. The OVV of claim 23, wherein the variant B5 polypeptide comprises an I236P substitution, a V238R substitution, a T240R substitution, and an E243G substitution.

34. The OVV of claim 23, wherein the variant B5 polypeptide comprises a V233D substitution, an I236L substitution, a V238W substitution, a T240Y substitution, and an E243R substitution.

35. The OVV of claim 23, wherein the variant B5 polypeptide comprises an D263V substitution, an E268T substitution, an E270G substitution, an E272P substitution, and an E275S substitution.

36. The OVV of claim 23, wherein the variant B5 polypeptide comprises an N241T substitution, an E243V substitution, a V247S substitution, a G250R substitution, and an A276F substitution.

37. The OVV of claim 23, wherein the variant B5 polypeptide comprises an N241G substitution, an E243S substitution, a V247W substitution, a D248Y substitution, a G250A substitution, and an A276F substitution.

38. The OVV of any one of claims 1 and 3-37, comprising a nucleotide sequence encoding a variant A34 polypeptide, wherein the variant A34 polypeptide comprises a K151E substitution

39. The OVV of any one of claims 1-38, further comprising a nucleotide sequence encoding a variant A56 polypeptide.

40. The OVV of claim 39, wherein the variant A56 polypeptide comprises a substitution of amino acid 269.

41. The OVV of claim 40, wherein the substitution of amino acid 269 is an I269F substitution.

42. The OVV of any one of claims 1-41, which comprises a heterologous nucleic acid comprising a nucleotide sequence encoding an immunomodulatory polypeptide.

43. The OVV of any one of claims 1-41, which comprises a modification that results in lack of J2R expression and/or function.

44. The OVV of any one of claims 1-41, which comprises both: i) a heterologous nucleic acid comprising a nucleotide sequence encoding an immunomodulatory polypeptide; and ii) a modification that results in lack of J2R expression and/or function.

45. The OVV of claims 1-44, which is constructed based on a strain of vaccinia virus selected from the group consisting of: Lister, New York City Board of Health, Wyeth, Copenhagen, Western Reserve, Vaccinia Ankara, EM63, Ikeda, Dalian, LIVP, Tian Tan, IHD-J, Tashkent, Bern, Paris, and Dairen.

46. A composition, comprising:
a) the OVV of any one of claims 1-45; and
b) a pharmaceutically acceptable excipient.

47. A method of inducing oncolysis in an individual having a tumor, comprising administering to the individual an effective amount of the OVV of any one of claims 1-45, or the composition of claim 46.

48. The method of claim 47, wherein said administering comprises administering a single dose of the OVV or the composition.

49. The method of claim 48, wherein the single dose comprises at least 10⁶ plaque forming units (pfu) of the OVV.

50. The method of claim 47, wherein said administering comprises administering multiple doses of the OVV or the composition.

51. The method of claim 50, wherein the OVV virus or the composition is administered every other day.

52. The method of claim 50, wherein the OVV virus or the composition is administered once per week.

53. The method of claim 50, wherein the OVV virus or the composition is administered every other week.

54. The method of any one of claims 47-53, wherein the tumor is a brain cancer tumor, a head and neck cancer tumor, an esophageal cancer tumor, a skin cancer tumor, a lung cancer tumor, a thymic cancer tumor, a stomach cancer tumor, a colon cancer tumor, a liver cancer tumor, an ovarian cancer tumor, a uterine cancer tumor, a bladder cancer tumor, a testicular cancer tumor, a rectal cancer tumor, a breast cancer tumor, or a pancreatic cancer tumor.

55. The method of any one of claims 47-53, wherein the tumor is colorectal cancer.

56. The method of any one of claims 47-53, wherein the tumor is non-small cell lung cancer.

57. The method of any one of claims 47-53, wherein the tumor is breast cancer.

58. The method of claim 57, wherein the tumor is a triple-negative breast cancer.

59. The method of any one of claims 47-58, wherein the tumor is a solid tumor.

60. The method of any one of claims 47-58, wherein the tumor is a liquid tumor.

61. The method of any one of claims 47-60, wherein the tumor is recurrent.

62. The method of any one of claims 47-60, wherein the tumor is a primary tumor.

63. The method of any one of claims 46-62, wherein the tumor is metastatic.

64. The method of any one of claims 46-63, further comprising administering to the individual a second cancer therapy.

65. The method of claim 64, wherein the second cancer therapy is selected from chemotherapy, biological therapy, radiotherapy, immunotherapy, hormone therapy, anti-vascular therapy, cryotherapy, toxin therapy, oncolytic virus therapy, a cell therapy, gene therapy, and surgery.

66. The method of claim 64, wherein the second cancer therapy is an immune checkpoint inhibitor.

67. The method of claim 66, wherein the immune checkpoint inhibitor is an antibody specific for an immune checkpoint inhibitor selected from CD27, CD28, CD40, CD122, CD96, CD73, CD47, OX40, GITR, CSF1R, JAK, PI3K delta, PI3K gamma, TAM, arginase, CD137, ICOS, A2AR, B7-H3, B7-H4, BTLA, CTLA-4, LAG3, TIM3, VISTA, CD96, TIGIT, CD122, PD-1, PD-L1 and PD-L2.

68. The method of any one of claims 47-67, wherein the individual is immunocompromised.

69. The method of any one of claims 47-68, wherein said administering of the OVV or the composition is intra-arterial, intraperitoneal, intrabladder, or intrathecal.

70. The method of any one of claims 47-68, wherein said administering of the OVV or the composition is intratumoral.

71. The method of any one of claims 47-68, wherein said administering of the OVV or the composition is peritumoral.

72. The method of any one of claims 47-68, wherein said administering of the OVV or the composition is intravenous.
